(19) United States
(12) Patent Application Publication
(10) Pub. No.: US 2021/0388388 A1
(43) Pub. Date: Dec. 16, 2021

(54) MUTANT VACCINIA VIRUSES AND USE THEREOF

(71) Applicant: ICELLKEALEX THERAPEUTICS LLC, Pearland, TX (US)

(72) Inventors: Xiaotong SONG, Pearland, TX (US); Mariya VISKOVSKA, Houston, TX (US); Maria Luiza GOMES MEDAGLIA, Houston, TX (US)

(21) Appl. No.: 17/287,497

(22) PCT Filed: Oct. 21, 2019
(86) PCT No.: PCT/US2019/057134

§ 371 Date: Apr. 21, 2021

Related U.S. Application Data
(60) Provisional application No. 62/749,102, filed on Oct. 22, 2018.
(60) Provisional application No. 62/912,344, filed on Oct. 8, 2019.

(51) Int. Cl. C12N 15/86; C12N 15/62; A61K 35/768; C07K 14/005

(52) U.S. Cl. CPC C12N 15/86; C12N 15/625; A61K 35/768; C12N 2710/24122; C12N 2710/24132; C12N 2710/24143; C07K 14/005

(57) ABSTRACT

The present invention discloses recombinant vaccinia virus (VV) virions that are resistant to antiviral defenses and have enhanced anti-tumor activities. In one embodiment, the recombinant VV comprise one or more variant VV proteins that have mutations at one or more neutralizing antibody epitopes, thereby conferring viral escape from the neutralizing antibodies. In another embodiment, the recombinant VV is resistant to complement-mediated neutralization due to the expression of a regulator of complement activation (e.g. CD55). In another embodiment, the recombinant VV has enhanced anti-tumor activities due to the expression of bi-specific antibodies co-targeting cancer cells and immune effector cells, or the expression of a polypeptide blocking the PD-1 pathway. The recombinant vaccinia virus virions can be used to treat cancer in a subject.

### CROSS-REFERENCE TO RELATED APPLICATIONS

This Patent Cooperation Treaty Application claims the benefit of priority of U.S. Provisional Patent Application No. 62/749,102, filed on Oct. 22, 2018, and U.S. Provisional Patent Application No. 62/912,344, filed on Oct. 8, 2019. See further description in Summary of The Invention.

### BACKGROUND OF THE INVENTION

Oncolytic viruses specifically infect, replicate in, and kill tumor cells while leaving normal cells undamaged. This preference for the transformed cells pegs oncolytic viruses as ideal candidates for the development of new cancer therapies. Various oncolytic viruses have been utilized to employ their tumor-specific killing activities by both direct (e.g. cell lysis due to viral replication and immune-mediated cytotoxicity), and indirect mechanisms (e.g. stimulation of the bystander cell killing, induction of cytotoxicity, etc). Oncolytic vaccinia virus (VV) is an appealing addition to the current treatment options, demonstrating efficacy and safety in animal models and in early clinical studies. In addition to infecting and killing tumor cells directly, VV may also induce a T-cell response against tumor antigens, increasing the efficiency of the killing. Whereas in some viruses this specificity toward cancer cells is naturally occurring (e.g. vesicular stomatitis virus, reovirus, mumps virus), other viruses can be genetically modified to improve their tumor specificity as well as to reduce their ability to induce antiviral immune response (e.g. adenovirus, measles virus, polio, and vaccinia virus). In addition, these viruses can be engineered to express genes that enhance antitumor immunity by recruitment of natural killer (NK) cells and T cells.

However, the effectiveness of oncolytic viruses is hindered by the strong immune response induced by the virus. Immune factors such as antibodies neutralize the virus by binding to it directly and preventing a successful infection of the cells or by marking it for destruction either by complement or by other immune cells. With each subsequent administration of the virus, the immune response is faster and stronger, which significantly restricts the ability of the virus to persist long enough to reach the tumor. A direct injection of the virus into the tumor overcomes this limitation and delivers all the viral particles directly to the cancer cells. However, this approach may not be suitable for some tumors and does not take into the account cases in which the tumors may have metastasized to other locations. A more desirable systemic administration of the virus exposes it to the host immune system capable of recognizing and eliminating potential pathogens. Immune factors such as neutralizing antibodies (NAbs) recognize and bind viral glycoproteins with high affinity and prevent virus interaction with host cell receptors, leading to virus neutralization. Several oncolytic viruses, such as adenovirus, herpes simplex virus, and vesicular stomatitis virus, have been genetically attenuated to placate their ability to induce antiviral defenses and improve tumor specificity.

Oncolytic vaccinia virus (VV) is the most studied member of the Poxviridae and is a large, enveloped, dsDNA virus. Strains highly specific to the tumor cells have been reported. VV's ability for rapid replication results in efficient lysis of infected cells as well as spread to other tumor cells upon successive rounds of replication, leading to profound localized destruction of the tumor. The VV genome encodes˜250 genes and can accept as much as 20 kb of foreign DNA, making it ideal as a gene delivery vehicle. The recombinant VV vectors are being developed to deliver eukaryotic genes, such as tumor-associated antigens, to the tumors and thus facilitate an induction of the host immune system directed to kill the cancer cells. However, a limiting factor in the use of VVs as cancer treatment delivery vectors is the strong NAb response induced by the injection of VV into the bloodstream that limits the ability of the virus to persist and spread and prevents vector re-dosing. The NAbs recognize and bind viral glycoproteins embedded in the VV envelope, thus preventing virus interaction with host cell receptors. A number of VV glycoproteins involved in host cell receptor recognition have been identified. Among them, proteins H3L, L1R, A27L, D8L, A33R, and B5R have been shown to be targeted by NAbs, with A27L, H3L, D8L and L1R being the main NAb antigens presented on the surface of mature viral particles. A27L, H3L, and D8L are the adhesion molecules that bind to host glycosaminoglycans (GAGs) heparan sulfate (HS) (A27L and H3L) and chondroitin sulfate (CS) (D8L) and mediate endocytosis of the virus into the host cell. L1R protein is involved in virus maturation.

Vaccinia virus is the prototype virus of the orthopoxvirus genus in the family Poxviridae, which replicates in the cytoplasm of cells and encodes more than 200 open reading frames (ORFs) in a 190-kb double-stranded DNA genome. Vaccinia virus infection produces multiple forms of infectious particles, namely, intracellular mature virions (IMV), intracellular enveloped virions (IEV), cell-associated enveloped virions (CEV), and extracellular enveloped virions (EEV). The IMV is the most abundant virion, with a single membrane in cells. IMVs are released only during cell lysis. Once released, IMVs efficiently infect neighboring cells via interactions between cell receptors and viral glycoproteins imbedded in the IMV membrane. A portion of the IMV is subsequently wrapped with two layers of Golgi membrane to form an IEV, which is transported through microtubules to the cell periphery and loses one membrane during virion egress to become a CEV. A small percentage (˜5%) of the IMVs is moved toward the cell's periphery where it acquires an outer envelope via fusion with the cell plasma membrane and is subsequently released into the extracellular space as an EEV. Thus, EEV is composed of the viral DNA core, the intermediate IMV, and an outermost membrane. This outer membrane is fragile and can be easily lost, thus EEVs are easily converted to the IMVs exposing the IMV imbedded antigens. The IMV is robust and is known to be resistant to environmental and physical changes, whereas the CEV and EEV are very fragile, and the integrity of their outer membranes can be destroyed during purification procedures.

Many of the poxvirus genomes, including those of different strains of vaccinia virus, have been sequenced. The genome of the vaccinia virus Western Reserve (WR) strain contains 218 potential ORFs. Analysis of the proteins in the IMV showed that it contains 81 viral proteins, including structural proteins, enzymes, transcription factors, etc. The 81 viral proteins in IMV are A2.5L, A3L, A4L, ASR, A6L, A7L, A9L, A10L, A12L, A13L, A14L, A14.5L, A15L, A16L, A17L, A18R, A21L, A22R, A24R, A25L, A26L, A27L, A28L, A29L, A30L, A31R, A32L, A42R, A45R, A46R, B1R, C6L, D1R, D2R, D6R, D7R, D8L, D11L, D12L, D13L, E1L, E4L, E6R, E8R, E10R, E11L, F8L, F9L, F10L, F17R, G1L, G3L, G4L, G5R, G5.5R, G7L, G9R, H1L, H2R, H3L, H4L, H5R, H6R, I1L, I2L, I3L, I5L, I6L, I7L, I8R, J1R, J3R, J4R, K4L, L1R, L3L, L4R, L5R, O2L. Among these proteins, A27L, H3L, L1R, and D8L have been identified as major immunogenic proteins. IMV proteins A27L, H3L, and D8L are the adhesion molecules that bind to host glycosaminoglycans (GAGs) heparan sulfate (HS) and chondroitin sulfate (CS) (D8L) and mediate endocytosis of the virus into the host cell. IMV L1R protein is involved in virus maturation. These proteins are the main immunodominant antigens on the IMV.

VV H3L is the membrane protein tethered to the membrane of the mature viral particles post-translationally via its hydrophobic region in the C-terminus. It is expressed late during the infection and, together with A27L, recognizes the HS cell surface receptors and plays a major role in VV adhesion to the cells. H3L is an immunodominant antigen in the anti-VV Ab response and a direct target of NAbs in humans immunized by the smallpox vaccine. Strong immune responses to H3L have also been shown in mice and rabbits. To date, the exact epitopes on H3L that are recognized by the NAbs have not been elucidated.

D8L is the VV envelope protein expressed early in infection and is involved in viral adhesion to host cells. While A27L and H3L interact with the HS host cell receptors, D8L binds to the CS receptors via its N-terminal domain (between residues 1-234). As one of the main viral antigens, D8L elicits a strong NAb response with the NAbs targeting the CS-binding region on the D8L and blocking viral adhesion to the cells. Several Abs targeting the D8L protein have been described. One of these Abs neutralized VV in the presence of a complement and targeted a conformational epitope on D8 (between residues 41 to 220). Residues R44, K48, K98, K108, and R220, a region adjacent to the CS binding site on D8L, are also important for Ab binding. In addition, N9, E30, T34, T35, N46, F47, K48, G49, G50, Y51, N59, E60, L63, S64, D75, Y76, H95, W96, N97, K99, Y101, S102, S103, Y104, E105, E106, K108, H110, D112, Q122, L124, D126, K163, T187, P188, and N190 have been identified as D8 antibody binding sites. It is not known whether mutation of these residues will confer sufficient escape from neutralization antibodies. Furthermore, whether mutations of these residues will impair virus packaging and cell entry due to D8L′s role in cell entry remain to be determined.

L1R is a transmembrane protein found on the surface of the mature VV particles. Its transmembrane domain lies in the C-terminal regions of the protein between residues 186 and 204. L1R is encoded by the L1R ORF, is highly conserved, and plays an essential role in viral entry and maturation. As one of the main targets of anti-VV NAb, L1R is included as a component of the poxvirus protein subunit and DNA vaccines. The NAb binding epitopes on the L1R protein have been characterized. An earlier study identified potent NAbs recognizing a linear epitope spanning residues 118-128 and a conformation epitope that partially overlapped with the linear peptide, specifically residues K125 and K127. A more recent study identified a group of 3 anti-L1R monoclonal Abs that potently neutralized VV in an isotype- and complement-independent manner. These NAbs recognized a conformational epitope with D35 as the key residue. Viral clones that contained a single amino acid mutation at residue D35 (either D35N or D35Y substitution) were completely resistant to neutralization by all Abs, indicating that D35 is essential for NAb recognition of L1R and binding. However, it is not clear if D35N will induce new neutralization antibody responses against 35N. In addition to D35, residues E25, N27, Q31, T32, K33, S58, D60, and D62 have been identified to be directly involved in binding with the Ab. It is not known whether mutations of these residues will escape neutralization antibody sufficiently and impair virus packaging and cell entry due to LIR's role in cell entry.

A27L is a 14-kDa protein in the envelope of the intracellular mature virus (IMV) that functions in viral host cell recognition and entry. It binds to the HS receptor on the host cell surface via its N-terminal domain (residues 21 to 30) and is attached to the VV envelope by interacting with the envelope protein A17 through its C-terminal domain. A recent study has identified several linear epitopes on the A27L that are recognized by the anti-A27L Abs. The Abs were categorized into four different groups with the Abs in group I binding to the peptide (residues 31 to 40) adjacent to the HS binding site and showing potent virus neutralization in the presence of complement. Crystal structures of the full-length A27L in a complex with these Abs identified residues E33, I35, V36, K37, and D39 to be critical for binding. Alanine substitutions of these residues resulted in the decreased ability of the Abs to bind to the peptide. A further analysis of the structures showed that residues K27, A30, R32, A34, E40, R107, P108, and Y109, although not critical, also contribute to the A27L-Ab binding.

In view of the above, there is a need for improved or genetically attenuated vaccinia viruses that have reduced ability to induce antiviral defenses and have enhanced anti-tumor activities. For example, ways to reduce induction of antiviral defenses and enhance anti-tumor activities include strategies for resisting neutralizing antibodies, overcoming complement-mediated virus neutralization, arming vaccinia viruses with bi-specific polypeptides to boost virus therapy, and/or incorporating immune checkpoint molecules to boost virus therapy.

### SUMMARY OF THE INVENTION

In one embodiment, the present invention provides mutant vaccinia viruses that are useful as viral vectors and vaccines.

Disclosed herein are recombinant vaccinia viruses comprising variant H3L, D8L, A27L and/or L1R viral proteins, including those of SEQ ID NOs:170 and 172. Further disclosed herein are recombinant vaccinia viruses comprising a heterologous nucleic acid encoding one of the following polypeptides: a domain of CD55 protein, a bi-specific polypeptide that binds to CD3e and FAP (fibroblast activation protein), a bi-specific polypeptide that binds to CD3e and BCMA (B-cell maturation antigen), and a fusion polypeptide comprising human PD-1 extracellular domain.

In one embodiment, the present invention provides mutant vaccinia viruses and uses thereof. In one embodiment, there is provided mutant vaccinia viruses having one or more mutation in the genes encoding proteins involved in binding neutralization antibodies or T cells. These mutations result in mutant vaccinia viruses having the ability to escape vaccinia virus-specific neutralization antibodies or T cells when compared to the wild-type virus.

In one embodiment, the present invention provides an isolated infectious recombinant vaccinia virus (VV) virion, the recombinant VV virion comprises a heterologous nucleic acid and one or more of:
(a) a variant vaccinia virus (VV) H3L protein that comprises an amino acid sequence having at least about 60%, 70%, 80%, 90%, or 95% amino acid sequence identity to SEQ ID NO:1;
(b) a variant vaccinia virus (VV) D8L protein that comprises an amino acid sequence having at least about 60%, 70%, 80%, 90%, or 95% amino acid sequence identity to SEQ ID NO:2;
(c) a variant vaccinia virus (VV) A27L protein that comprises an amino acid sequence having at least about 60%, 70%, 80%, 90%, or 95% amino acid sequence identity to SEQ ID NO:3;
(d) a variant vaccinia virus (VV) L1R protein that comprises an amino acid sequence having at least about 60%, 70%, 80%, 90%, or 95% amino acid sequence identity to SEQ ID NO:4;
(e) a variant vaccinia virus (VV) H3L protein that comprises an amino acid sequence having at least about 60%, 70%, 80%, 90%, or 95% amino acid sequence identity to SEQ ID NO:5;
(f) a variant vaccinia virus (VV) D8L protein that comprises an amino acid sequence having at least about 60%, 70%, 80%, 90%, or 95% amino acid sequence identity to SEQ ID NO:6 or SEQ ID NO:174;
(g) a variant vaccinia virus (VV) H3L protein that comprises an amino acid sequence having at least about 60%, 70%, 80%, 90%, or 95% amino acid sequence identity to SEQ ID NO:170; and
(h) a variant vaccinia virus (VV) D8L protein that comprises an amino acid sequence having at least about 60%, 70%, 80%, 90%, or 95% amino acid sequence identity to SEQ ID NO:172.

In one embodiment, the present invention provides recombinant vaccinia virus (VV) virions comprising a nucleic acid encoding a complement activation modulator such as part or all of CD55, CD59, CD46, CD35, factor H, and C4-binding protein, and the like, and uses thereof. Expression of the complement activation modulators results in recombinant vaccinia viruses having the ability to modulate complement activation and reduce complement-mediated virus neutralization when compared to the wild-type virus. In one embodiment, the CD55 protein comprises the amino acid sequence of SEQ ID NO:7.

In one embodiment, the present invention provides recombinant vaccinia virus (VV) virions comprising a bi-specific FAP-CD3 scFv that comprises an amino acid sequence having the sequence of SEQ ID NO:8.

In one embodiment, the present invention provides recombinant vaccinia virus (VV) virions comprising a bi-specific BCMA-CD3 scFv that comprises an amino acid sequence having the sequence of SEQ ID NO:9.

In one embodiment, the present invention provides recombinant vaccinia virus (VV) virions comprising a PD-1-ED-hIgG1-Fc fusion peptide that comprises an amino acid sequence having the sequence of SEQ ID NO:10.

In another embodiment, the present invention provides a method of delivering a gene product to an individual in need thereof, the method comprising administering to the individual an effective amount of an infectious recombinant vaccinia virus (VV) virion disclosed herein, wherein the gene product is encoded by the heterologous nucleic acid carried by the recombinant VV virion.

In one embodiment, there is provided a pharmaceutical composition comprising the recombinant vaccinia virus (VV) virion disclosed herein, and methods of using such composition to treat cancer.

In one embodiment, there is provided a library comprising one or more variant vaccinia virus (VV) virions, each of said variant VV virions comprises one or more variant VV protein, the variant VV protein comprises an amino acid sequence having at least one amino acid substitution relative to the amino acid sequence of a corresponding wild type VV protein.

In another embodiment, the present invention provides a method of delivering a gene product to an individual in need thereof, the method comprises administering to the individual an effective amount of infectious variant vaccinia virus (VV) virions derived from the above library, wherein the gene product is encoded by a nucleic acid carried by such variant VV virions.

In another embodiment, there is provided a pharmaceutical composition comprising variant vaccinia virus (VV) virions derived from the above library, and methods of using such composition to treat cancer.

In one embodiment, there is provided a recombinant vaccinia virus H3L protein that has at least about 60%, 70%, 80%, 90%, or 95% amino acid sequence identity to one of SEQ ID NOs:1, 5 or 170. In another embodiment, there is provided a recombinant vaccinia virus D8L protein that has at least about 60%, 70%, 80%, 90%, or 95% amino acid sequence identity to SEQ ID NOs:6, 172 or 174.

### BRIEF DESCRIPTION OF THE DRAWINGS

FIGS. 1A-C show neutralizing antibody (Nab) epitope determination of H3L -peptide arrays sequence analysis. Antibody 35219 was used for binding to the peptide array of the H3L sequence (Ab35219 is a rabbit polyclonal to VV; Immunogen: Native virus, Lister strain).

FIG. 1A shows diagram of the SPOT-synthesis peptide array. FIG. 1B shows autoradiograph of the H3L peptide array probed by ab35219. The peptide array consists of spots of 12-residue peptides in the H3L sequence, starting from the N terminus (spot 1) and ending with the C-terminal peptide (spot 69), with the N-terminal residue of the peptide in each spot shifted by 4 residues from the previous spot along the H3L sequence. FIG. 1C are graphs showing signal intensity (y axis) of each spot (black bars) (x axis).

FIGS. 2A-B show NAb epitope mapping of H3L by linear peptide ELISA. FIG. 2A shows ELISA results for H3L peptides 1-4. FIG. 2B shows ELISA results for H3L peptides 5-9. Arrows indicate some examples of alanine-substituted residues that have an effect on antibody (Ab) binding. Alanine scan identified total of 29 residues positive for Ab binding: I14A, D15A, R16A, K33A, F34A, D35A, K38A, N40A, E45A, V52A, E131A, T134A, F135A, L136A, R137A, R154A, E155A, I156A, K161A, L166A, V167A, M168A, I198A, R227A, E250A, K253A, P254A, N255A, and F256A. A lower optical density (OD) indicates that the alanine-substituted peptide preincubated with the Ab binds sufficiently to prevent the Ab binding to plate-bound native peptide. A higher OD (arrows) indicates the decreased ability of the mutant peptide to interact with the Ab, signifying that the mutated residue is important for H3L binding to Ab.

FIGS. 3A-D show construction of modified H3L, D8L, L1R, and A27L plasmids.

FIG. 3A shows a construct containing the H3L promoter, H3L ORF (with mutated nucleotides), and approximately ˜250-bp flanking regions containing the H4L (left flank) and the H2R (right flank) ORF sequences was synthesized by GENEWIZ and cloned into the pUC57-Amp plasmid.

FIG. 3B shows a construct containing the D8L promoter, D8L ORF (with mutated nucleotides), and approximately ˜250-bp flanking regions containing the D9R (left flank) and the D7R (right flank) ORF sequences was synthesized by GENEWIZ and cloned into the pUC57-Amp plasmid.

FIG. 3C shows a construct containing the L1R promoter, L1R ORF (with mutated nucleotides), and approximately ˜250-bp flanking regions containing the G9R (left flank) and the L2R (right flank) ORF sequences was synthesized by GENEWIZ and cloned into the pUC57-Amp plasmid.

FIG. 3D shows a construct containing the A27L promoter, A27L ORF (with mutated nucleotides), and approximately ˜250-bp flanking regions containing the A28-A29L (left flank) and the A26L (right flank) ORF sequences was synthesized by GENEWIZ and cloned into the pUC57-Amp plasmid. For all four constructs a green fluorescent protein (GFP) expression cassette under the control of the VV p7.5 promoter and flanked by LoxP sites was inserted immediately downstream of the stop codon before the right flank sequence.

FIG. 4 shows identification of the correct H3L, D8L, L1R, and A27L recombinant clones. Single plaques were purified and correct gene insertions were confirmed by PCR.

FIG. 5 shows plaque reduction neutralization tests (PRNTs) using polyclonal anti-VV Abs. A panel of five anti-VV polyclonal antibodies consisting of ab35219 (Abcam)—rabbit polyclonal to VV (Immunogen: Native virus, Lister strain), ab21039 (Abcam)—rabbit polyclonal to VV (Immunogen: Lister Strain (mixture of virions and infected cell polypeptides)), ab26853 (Abcam)—rabbit polyclonal to VV (Immunogen: Synthetic peptide containing amino acids on the predicted N terminus of A27L in VV), 9503-2057 (Bio-Rad)—rabbit polyclonal against VV Ab (Immunogen: Vaccinia virus, New York City Board of Health (NYCBOH) strain), and PA1-7258 (Invitrogen)—rabbit polyclonal against VV (Immunogen: NYCBOH strain and Lister strain) was used to test for neutralization escape in vitro. Rabbit polyclonal IgG ab37415 served as a control. Abs were preincubated with either the escape variant or the wt VV virus (control) in the presence of sterile baby rabbit complement. The mixture was then added to the CV-1 cells and 48 hrs later cells were stained and plaques counted. Whereas 83.3-95.5% of the control VV virus was neutralized across the panel, the escape variant (FAP-VVNEV) showed a significantly lower neutralization by the Abs (7.88-66.1%). Error bars are based on two or three data points per sample.

FIG. 6 shows VV^{EM} (vaccinia virus escape mutant) in vitro plaque reduction neutralization test with anti-VV polyclonal Abs. VV^{EM} was isolated from the mutant VV library pool in the presence of anti-VV polyclonal antibodies. A panel of five anti-VV polyclonal antibodies consisting of ab35219, ab21039, ab26853, 9503-2057, and PA1-7258 was used to test VV^{EM} for neutralization escape in vitro. Rabbit polyclonal IgG ab37415 served as a control. Abs were preincubated with either the VV^{EM} or the wild type VV virus (control) in the presence of sterile baby rabbit complement. Whereas 77.7-96.4% of the control VV virus was neutralized across the panel, VV^{EM} showed a significantly lower (30.7-66.9%) neutralization by the Abs. Error bars are based on two or three data points per sample. VV^{EM} was further sequenced to identify the mutation within H3, L1, A27, or D8 that might be responsible for the Nab escape.

FIG. 7 shows results of a recombinant virus replication assay. In a 24-well plate CV-1 cells were infected with duplicates of VV control and VV^{NEV} at MOI=0.05. Prior to infection virus was preincubated with Ab 9503-2057 (40 μg/mL) for 1 hour at 37° C. Samples were collected at 24, 48, and 72 hours and titers were determined for each time point. The recombinant virus was significantly more efficient in replicating in the presence of Ab, compared to the control Ab, which was almost entirely inactivated.

FIG. 8 shows anti-tumor efficiency of the recombinant virus. The recombinant virus and the control VV were preincubated with Ab 9503-2057 (see above) and used to infect transformed cells at MOI=1. Cells were incubated for 48 hours and cell viability was measured by MTS assay (colorimetric assessment of cell metabolic activity). Briefly, cells collected at 48 hours were washed once with PBST and resuspended at 1×105 cells/mL in complete DMEM. One hundred μL of each cell suspension was added to a 96-well (in triplicates). Twenty μl of CellTiter 96® AQueous One Solution Reagent (Promega, G358C) was added into each well of the 96-well assay plate containing the samples in 100 μl of culture medium. The plate was incubated at 37° C. for 2 hours (5% CO2). To measure the amount of soluble formazan produced by cellular reduction of MTS, the absorbance in each well was recorded at 490 nm using a 96-well plate reader. In the presence of the Ab, the recombinant virus was able to efficiently kill the cells.

FIG. 9 shows a recombinant VV^{NEV} in vitro plaque reduction neutralization test with anti-VV polyclonal Abs. Anti-VV polyclonal antibodies 9503-2057 and PA1-7258 were used to test VV^{EM} for neutralization escape in vitro. Rabbit polyclonal IgG ab37415 served as a control. Abs were preincubated with either the VV^{NEV} (right panel) or the wild type vaccinia virus (control, left panel) in the presence of sterile baby rabbit complement.

FIG. 10 shows results of a recombinant virus replication assay. In a 24-well plate CV-1 cells were infected with duplicates of VV control and 3 single clones of VV^{NEV} at MOI=0.05. Samples were collected at 24, 48, and 72 hours and titers were determined for each time point.

FIG. 11 shows a CD55-A27-VV construct containing the A27 promoter, CD55-ED, A27, loxP-flanked tag, and flanking regions containing the A27L (left flank) and the A27R (right flank). ORF sequences was synthesized by GENEWIZ and cloned into the pUC57-Amp plasmid.

FIG. 12 shows CD55-NEV escapes complement-mediated neutralization effectively in vitro.

FIG. 13 shows CD55-NEV escapes neutralization antibody and complement-mediated neutralization effectively in vitro.

FIG. 14 shows a FAP-TEA-NEV construct containing the F 17R promoter, FAP-CD3 scFv, loxP-flanked tag, and flanking regions containing the TKL (left flank) and the TKR (right flank). ORF sequences was synthesized by GENEWIZ and cloned into the pUC57-Amp plasmid.

FIG. 15 shows a FAP-TEA-NEV enhanced tumor lysis and human T cell proliferation in vitro (see circle, microscopy observation).

FIG. 16 shows a FAP-TEA-NEV induced tumor cell apoptosis effectively (flow cytometry analysis).

FIG. 17 shows MFI of apoptosis marker PI staining of gated U87 tumor cells.

FIG. 18 shows a bispecific FAP-CD3 scFv expressed by FAP-TEA-NEV enhanced bystander tumor lysis in vitro (see circles, microscopy observation).

FIG. 19 shows a BCMA-TEA-NEV construct containing the F17 promoter, BCMA-CD3 scFv, loxP-flanked GFP-tag, and flanking regions containing the TKL (left flank) and the TKR (right flank). ORF sequences was synthesized by GENEWIZ and cloned into the pUC57-Amp plasmid.

FIGS. 20A-B show flow cytometric analysis of co-culture of BCMA-positive RMPI-8226 MM and Jurkat T cells.

FIGS. 21A-B show ELISA measurement of IFNy and IL2 expression by Jurkat T cells following 24 hours co-culture with BCMA-positive RMPI-8226 MM.

FIG. 22 shows a PD-1-ED-hIgG1-Fc-VV construct containing the pE/L promoter, PD-1-ED-hIgG1-Fc, loxP-flanked GFP-tag, and flanking regions containing the TKL (left flank) and the TKR (right flank). A PD-1-ED-hIgG1-Fc-FAP-TEA-NEV construct containing the pE/L promoter, PD-1-ED-hIgG1-Fc, F17R promoter, FAP-CD3 scFv, loxP-flanked GFP-tag, and flanking regions containing the TKL (left flank) and the TKR (right flank) is also shown. ORF sequences was synthesized by GENEWIZ and cloned into the pUC57-Amp plasmid.

FIGS. 23A-B show flow cytometric analysis of co-culture of PD-L1-positive Raji cells and CD16-positive Jurkat T cells.

FIG. 24A-B show ELISA measurement of IFNγ and IL2 expression by CD16-positive Jurkat T cells following 24 hours co-culture with PD-L1-positive Raji cells.

FIG. 25 shows the luciferase activity measurement of CD16-positive Jurkat T cells following 24 hours co-culture with PD-L1-positive Raji cells.

### DETAILED DESCRIPTION OF THE INVENTION

The present invention discloses the making and uses of variant vaccinia virus (VV) virions that have reduced ability to induce antiviral defenses and have enhanced anti-tumor activities.

### Enhancing Resistance to Neutralizing Antibodies

In one embodiment, the variant vaccinia virus (VV) virions of the present invention have increased resistance to anti-VV neutralizing antibodies. For example, the variant vaccinia virus virions of the present invention comprise one or more variant VV proteins (such as H3L protein, D8L protein, A27L protein, and L1R protein) that have mutations at one or more neutralizing antibody epitopes, thereby conferring viral escape from the neutralizing antibodies.

The present specification discloses experiments studying variant VV protein H3L. The same experimental setup can be used to study other vaccinia virus viral proteins such as D8L protein, A27L protein, L1R protein etc. To identify possible regions on the viral protein that interact with neutralizing antibodies, peptide arrays encompassing the full-length viral protein was synthesized and screened for peptides that bound the anti-VV neutralizing antibodies. Peptides thus identified were further examined to elucidate the neutralizing antibody epitopes. In one embodiment, variants of the peptides identified by the peptide array were synthesized with alanine substitutions, and the neutralizing antibody epitopes were mapped using a series of ELISA binding assays. Once the neutralizing antibody epitopes were identified, mutations that destroy these epitopes can be introduced into the VV genome by genetic engineering.

The present invention discloses a number of neutralizing antibody epitopes on each of the vaccinia virus H3L protein, D8L protein, A27L protein, and L1R protein. Mutating or substituting amino acid(s) at these neutralizing antibody epitopes would confer viral escape from the neutralizing antibodies. Similarly, deleting amino acid(s) at these neutralizing antibody epitopes is also expected to confer viral escape from the neutralizing antibodies. Hence, it is expected that deletion of one or more amino acids within the H3L, D28L, A27L, L1R viral protein, or deletion of the whole H3L, D28L, A27L, or L1R viral protein could also confer escape from neutralizing antibody binding. H3L deletion mutant variants have been reported, indicating the feasibility of generating one or more amino acid deletion or whole protein deletion virus mutants, even though the H3L deletion impaired the virus mutant's infectivity and replication capability.

In one embodiment, the present invention provides an isolated infectious recombinant vaccinia virus (VV) virion, comprising a heterologous nucleic acid and one or more of:
a) a variant vaccinia virus (VV) H3L protein that comprises an amino acid sequence having at least about 60%, 70%, 80%, 90%, or 95% amino acid sequence identity to SEQ ID NO:1;
b) a variant vaccinia virus (VV) D8L protein that comprises an amino acid sequence having at least about 60%, 70%, 80%, 90%, or 95% amino acid sequence identity to SEQ ID NO:2;
c) a variant vaccinia virus (VV) A27L protein that comprises an amino acid sequence having at least about 60%, 70%, 80%, 90%, or 95% amino acid sequence identity to SEQ ID NO:3;
d) a variant vaccinia virus (VV) L1R protein that comprises an amino acid sequence having at least about 60%, 70%, 80%, 90%, or 95% amino acid sequence identity to SEQ ID NO:4;
e) a variant vaccinia virus (VV) H3L protein that comprises an amino acid sequence having at least about 60%, 70%, 80%, 90%, or 95% amino acid sequence identity to SEQ ID NO:5;
f) a variant vaccinia virus (VV) D8L protein that comprises an amino acid sequence having at least about 60%, 70%, 80%, 90%, or 95% amino acid sequence identity to SEQ ID NO:6 or SEQ ID NO:174;
g) a variant vaccinia virus (VV) H3L protein that comprises an amino acid sequence having at least about 60%, 70%, 80%, 90%, or 95% amino acid sequence identity to SEQ ID NO:170; and
h) a variant vaccinia virus (VV) D8L protein that comprises an amino acid sequence having at least about 60%, 70%, 80%, 90%, or 95% amino acid sequence identity to SEQ ID NO:172.

In one embodiment, the above variant VV H3L protein comprises amino acid substitution or deletion at one or more of the following amino acid residues: 14, 15, 16, 33, 34, 35, 38, 40, 44, 45, 52, 131, 134, 135, 136, 137, 154, 155, 156, 161, 166, 167, 168, 198, 227, 250, 253, 254, 255, and 256 of SEQ ID NO:1. Any suitable amino acids can be used in the substitutions. For example, variant peptides can be synthesized with substitutions.

In one embodiment, the above variant VV D8L protein comprises amino acid substitution or deletion at one or more of the following amino acid residues: 44, 48, 98, 108, 117, and 220 of SEQ ID NO:2. Any suitable amino acids can be used in the substitutions. For example, variant peptides can be synthesized with substitutions.

In one embodiment, the above variant VV A27L protein comprises amino acid substitution or deletion at one or more of the following amino acid residues: 27, 30, 32, 33, 34, 35, 36, 37, 39, 40, 107, 108, and 109 of SEQ ID NO:3. Any suitable amino acids can be used in the substitutions. For example, variant peptides can be synthesized with substitutions.

In one embodiment, the above variant VV L1R protein comprises amino acid substitution or deletion at one or more of the following amino acid residues: 25, 27, 31, 32, 33, 35, 58, 60, 62, 125, and 127 of SEQ ID NO:4. Any suitable amino acids can be used in the substitutions. For example, variant peptides can be synthesized with substitutions.

In one embodiment, the above variant VV H3L protein comprises amino acid substitution or deletion at one or more of the following amino acid residues: 14, 15, 16, 33, 34, 35, 38, 40, 44, 45, 52, 131, 132, 134, 135, 136, 137, 154, 155, 156, 161, 166, 167, 168, 195, 198, 199, 227, 250, 251, 252, 253, 254, 255, 256, 258, 262, 264, 266, 268, 272, 273, 275, and 277 of SEQ ID NO:170. Any suitable amino acids can be used in the substitutions.

In one embodiment, the above variant VV D8L protein comprises amino acid substitution or deletion at one or more of the following amino acid residues: 43, 44, 48, 53, 54, 55, 98, 108, 109, 144, 168, 177, 196, 199, 203, 207, 212, 218, 220, 222, and 227 of SEQ ID NO:172. Any suitable amino acids can be used in the substitutions.

### Overcoming Complement-Mediated Virus Neutralization

Complement is a key component of the innate immune system, targeting the virus for neutralization and clearance from the circulatory system. Complement could enhance neutralization antibody's neutralizing efficacy, and antibody-mediated protective immunity induced by smallpox vaccination was largely decreased in vitro in the absence of complement, indicating the critical role of complement in the neutralization of vaccinia virus. Complement activation results in cleavage and activation of C3 and deposition of opsonic C3 fragments on surfaces. Subsequent cleavage of C5 leads to assembly of the membrane attack complex (C5b, 6, 7, 8, 9), which disrupts lipid bilayers.

Complement activation can be negatively regulated by several membrane regulator of complement activation (RCA). RCAs downregulate complement activation at different steps. First, CD35 (complement receptor 1) and CD55 (decay-accelerating factor) inhibit the formation and accelerate the decay of C3 convertases (C3-activating enzymes). Second, CD35 and CD46 (membrane cofactor protein) catabolizes C4b and C3b, inhibiting formation of the C3 convertases C4b2a and C3bBb. Third, CD59 prevents the formation of the membrane attack complex. Studies have shown that extracellular enveloped vaccinia virus (EEV) is resistant to complement because of incorporation of host RCA into its envelope. However, it is not known whether CD55 and/or other RCAs can be successfully expressed on the surface of the IMV of VV with the ability of overcoming complement-mediated neutralization, without affecting viral packaging and replication.

In one embodiment, the present invention provides recombinant vaccinia virus (VV) virions comprising a heterologous nucleic acid encoding a complement activation modulator such as CD55, CD59, CD46, CD35, factor H, C4-binding protein, or other identified complement activation modulators, and uses thereof. Expressing the complement activation modulators results in recombinant vaccinia viruses having the ability to modulate complement activation and reduce complement-mediated virus neutralization as compared to the wild-type virus. In one embodiment, the heterologous nucleic acid carried by the above recombinant vaccinia virus (VV) virion encodes a domain of human CD55, CD59, CD46, CD35, factor H, C4-binding protein, or other identified complement activation modulators. In another embodiment, the heterologous nucleic acid encodes a CD55 protein that comprises an amino acid sequence having the sequence of SEQ ID NO:7. In view of the disclosure presented herein, one of ordinary skill in the art would readily employ other complement activation modulators (e.g. CD59, CD46, CD35, factor H, C4-binding protein etc) in the recombinant vaccinia virus presented herein.

### Incorporating Bi-Specific Antibodies to Boost Virus Therapy

Oncolytic virus can be armed to express bi-specific antibodies that bind to a first antigen on immune cells and a second antigen on tumor cells. Examples of the first antigen on immune cells include, but are not limited to, CD3, CD4, CD5, CD8, CD16, CD28, CD40, CD64, CD89, CD134, CD137, NKp46, and NKG2D, and the like. Examples of the second antigen on tumor cells include, but are not limited to, EphA2, HER2, GD2, Glypican-3, 5T4, 8H9, avb6 integrin, B7-H3, B7-H6, BCMA, CADC, CA9, CD19, CD20, CD22, kappa light chain, CD30, CD33, CD38, CD44, CD44v6, CD44v7/8, CD70, CD123, CD138, CD171, CEA, CSPG4, EGFR, EGFRv111, EGP2, EGP40, EPCAM, ERBB3, ERBB4, ErbB3/4, FAP, FAR, FBP, fetal AchR, Folate Receptor a, GD2, GD3, HLA-AI MAGE Al, HLA-A2, IL11Ra, IL13Ra2, KDR, Lambda, Lewis-Y, MCSP, Mesothelin, Mucl, Muc16, NCAM, NKG2D ligands, NY-ESO-1, PRAME, PSCA, PSC1, PSMA, RORI, SURVIVIN, TAG72, TEM1, TEM8, VEGRR2, carcinoembryonic antigen, HMW-MAA, VEGF receptors, and other exemplary antigens that are present within the extracellular matrix of tumors, such as oncofetal variants of fibronectin, tenascin, or necrotic regions of tumors.

### Targeting B-Cell Maturation Antigen (BCMA) to Treat Multiple Myeloma

Multiple myeloma (MM) is a malignancy of clonal plasma cells derived from the B-lymphocyte lineage that is part of a spectrum of diseases ranging from monoclonal gammopathy of undetermined significance (MGUS) to plasma cell leukemia. It is the second most common hematological cancer in the United States with an estimated 32,110 newly diagnosed cases and 12,960 deaths in 2019. MM currently accounts for 10% of hematological malignancies and 2.1% of all cancer-related deaths. Currently several treatments for MM are available, however no curative therapies have been defined and most patients will eventually relapse with a median survival of 3-5 years, regardless of treatment regimen or initial responses to treatment. Therapeutics with new mechanisms of action are therefore urgently needed to treat drug-resistant MM.

Oncolytic vaccinia virus (VV) emerged as a promising new class of agents with great potential for the treatment of MM. Live VV has been administered by WHO to over 200 million people to eradicate smallpox, giving VV an excellent history of safety in humans. While wild type VV has no tumor selectivity, double deletion of viral genes that are essential for viral replication in normal cells, such as thymidine kinase (TK) and vaccinia growth factor (VGF), have conferred a strict VV tumor specificity. Recent clinical trials of VV against solid tumors are reporting promising results. In vitro studies utilizing a strain double deleted for TK and VGF showed that MM cell lines are susceptible to killing by VV. In those studies, viral replication was observed in primary MM cells, but not in normal peripheral blood mononuclear cells (PBMCs). The double deleted strain also reduced tumor volume and increased survival in a mouse xenograft model of MM. In addition, recently a TK-deleted VV strain that overexpresses two anti-tumor factors, miR-34a and Smac (frequently dysregulated in MM) showed increased efficacy against MM both in vitro and in vivo when compared to treatment with the parental virus, VV-miR-34a, or VV-Smac individually. However, the efficacy of VV therapy in current clinical studies is not optimized, indicating the need of further improvement of VV therapy.

VV can express T-cell engager targeting or co-targeting MM antigens, such as BCMA, CD19, CD26, CD38, CD44v6, CD56, CD138, CS1, EGFR, integrin beta7, KIRs, LIGHT/TNFSF14, NKG2D, PD-1/PD-L1, SLAMF7, TACI, and TGIT. B-cell maturation antigen (BCMA), a transmembrane glycoprotein in the tumor necrosis factor receptor superfamily 17 (TNFRSF17), is a promising target for MM therapy because it is expressed at significantly higher levels in all patient MM cells but not in normal tissues, except in plasma cells (PC). In recent clinical studies BCMA-targeted chimeric antigen receptor (CAR) T-cells showed significant clinical activities in patients with relapsed and refractory multiple myeloma (RRMM) who have undergone at least three prior treatments, including a proteasome inhibitor and an immunomodulatory agent. Anti-BCMA Ab-drug conjugate (ADC) also has achieved significant clinical responses in patients who failed at least three prior lines of therapy. Both BCMA-targeted CAR-T and ADC were granted breakthrough status for patients with RRMM by FDA in November 2017. As promising as these two therapies are there are several complicating factors for targeting BCMA. First, anti-BCMA treatment will potentially reduce the number of long-lived PCs and, since long-lived PCs play a critical role in maintaining humoral immunity, the impact of anti-BCMA therapy on immune function needs to be carefully and serially evaluated. Second, high serum levels of sBCMA, cleaved from BCMA by γ-secretase have been detected in MM patients, especially in the setting of progressive disease. Thus, it is necessary to develop a therapeutic strategy to deliver the BCMA-targeted treatment directly to BCMA+ MM cells.

As described herein, the present invention provides recombinant vaccinia virus (VV), BCMA-TEA-NEV, that overcomes the limitations discussed above because the BCMA-CD3 BiTE expression will be limited within the MM surrounding area while escaping the BCMA+ PCs and sBCMA. TEA-NEV encodes bi-specific scFvs that directs T cells to recognize and kill tumor cells that are not infected with VV (by-stander killing), resulting in enhanced tumor lysis. In addition, the CD3-scFv promotes T-cell infiltration into tumors and their activation, and the cytokines they release upon activation create a pro-inflammatory micro-environment that inhibites tumor growth. In addition, the TEA-NEV induces local production of T-cell engager that allows for higher concentrations of T cells at the target site while reducing systemic side effects. Thus, arming oncolytic VV with bi-specific scFvs is important to engage T cells for cancer therapy and produce the desired increase in anti-tumor activity of current VV by inducing by-stander killing.

In one embodiment, the heterologous nucleic acid carried by the above recombinant vaccinia virus (VV) virion encodes a bi-specific polypeptide that binds to a first antigen on immune cells and a second antigen, B-cell maturation antigen (BCMA), on multiple myeloma (MM). For example, the bi-specific polypeptide is a bi-specific scFvs, the first antigen is human CD3e, the second antigen is human BCMA (B-cell maturation antigen), and the bi-specific scFvs comprises an amino acid sequence of SEQ ID NO:9.

In another embodiment, VV can express T-cell engager targeting or co-targeting other MM antigens, such as CD19, CD38, SLAMF7, CD26, LIGHT/TNFSF14, integrin beta7, CD138, KIRs, EGFR, PD-1/PD-L1, TGIT, CD56, CS1, NKG2D, TACI, and CD44v6.

In another embodiment, the bi-specific polypeptide is a bi-specific scFvs, the first antigen is human CD3e and the second antigen is human FAP (fibroblast activation protein) that is overexpressed on most epithelial cancers. In one embodiment, the bi-specific FAP-CD3 scFv comprises the amino acid sequence of SEQ ID NO:8.

### Incorporating Immune Checkpoint Molecules to Boost Virus Therapy

Increasing evidence has shown that T-cell immunotherapy has the ability to control tumor growth and prolong survival in cancer patients. However, tumor-specific T-cell responses are hard to achieve and sustain, likely due to the limitations of various immune escape mechanisms of tumor cells. Immune checkpoint molecules are proteins expressed on certain immune cells that need to be activated or inhibited to start an immune response, for example, to attack abnormal cells such as tumor cells in the body. The “immune escape” may include several activities by the tumor cells, such as down-regulation of co-stimulatory molecule expression, such as stimulatory immune checkpoint molecules, and up-regulation of inhibitory molecule expression, such as inhibitory immune checkpoint molecules. Blockade of these inhibitory immune checkpoint molecules have shown very promising results in preclinical and clinical tests in cancer treatment. However, there are some unwanted side effects in some cases. For example, blocking these inhibitory immune checkpoint molecules (receptors or ligands) may lead to a disruption in immune homeostasis and self-tolerance, resulting in autoimmune/auto-inflammatory side effects.

Immune checkpoint molecules are well-known in the art. For example, the PD-1 (programmed cell death-1) receptor is expressed on the surface of activated T cells. Its ligands, PD-L1 and PD-L2, are commonly expressed on the surface of dendritic cells or tumor cells. PD-1 and PD-L1/PD-L2 belong to the family of inhibitory immune checkpoint proteins that can halt or limit the development of T cell response. PD-L1 expressed on the tumor cells could bind to PD-1 receptors on the activated T cells, which leads to inhibition of cytotoxic T cells. Hence, anti-tumor immune responses would be enhanced by blocking the interaction between PD-1 and its ligands.

In one embodiment, the present invention provides recombinant vaccinia virus (VV) virions that would block the inhibitory PD-1 pathway. In one embodiment, the present invention provides recombinant vaccinia virus (VV) virions comprising a heterologous nucleic acid encoding an extracellular domain of PD-1 fused to the constant (Fc) domain of immunoglobin-G1 (IgG1). In one embodiment, the PD-1 fusion protein (PD-1-ED-hIgG1-Fc) comprises the amino acid sequence of SEQ ID NO:10. In view of the disclosure presented herein, other immune checkpoint molecules can be readily incorporated into the recombinant vaccinia virus presented herein. The recombinant vaccinia viruses disclosed herein may comprise immune checkpoint molecules including, but not limited to, PD-1, PD-L1, PD-L2, CD47, CXCR4, CSF1R, LAG-3, TIM-3, HHLA2, BTLA, CTLA-4, TIGIT, VISTA, B7-H4, CD160, 2B4, and CD73.

In one embodiment, the present invention provides an isolated infectious recombinant vaccinia virus (VV) virion, the virion comprises a heterologous nucleic acid and one or more of:
a) a variant vaccinia virus (VV) H3L protein having at least about 60% amino acid sequence identity to SEQ ID NO:1;
b) a variant vaccinia virus (VV) D8L protein having at least about 60% amino acid sequence identity to SEQ ID NO:2;
c) a variant vaccinia virus (VV) A27L protein having at least about 60% amino acid sequence identity to SEQ ID NO:3;
d) a variant vaccinia virus (VV) L1R protein having at least about 60% amino acid sequence identity to SEQ ID NO:4;
e) a variant vaccinia virus (VV) H3L protein having at least about 60% amino acid sequence identity to SEQ ID NO:5;
f) a variant vaccinia virus (VV) D8L protein having at least about 60% amino acid sequence identity to SEQ ID NO:6 or SEQ ID NO:174;
g) a variant vaccinia virus (VV) H3L protein having at least about 60% amino acid sequence identity to SEQ ID NO:170; and
h) a variant vaccinia virus (VV) D8L protein having at least about 60% amino acid sequence identity to SEQ ID NO:172.

In one embodiment, the variant VV H3L protein comprises amino acid substitution or deletion at one or more of the following amino acid residues: 14, 15, 16, 33, 34, 35, 38, 40, 44, 45, 52, 131, 134, 135, 136, 137, 154, 155, 156, 161, 166, 167, 168, 198, 227, 250, 253, 254, 255, and 256 of SEQ ID NO:1.

In one embodiment, the variant VV D8L protein comprises amino acid substitution or deletion at one or more of the following amino acid residues: 44, 48, 98, 108, 117, and 220 of SEQ ID NO:2.

In one embodiment, the variant VV A27L protein comprises amino acid substitution or deletion at one or more of the following amino acid residues: 27, 30, 32, 33, 34, 35, 36, 37, 39, 40, 107, 108, and 109 of SEQ ID NO:3.

In one embodiment, the variant VV L1R protein comprises amino acid substitution or deletion at one or more of the following amino acid residues: 25, 27, 31, 32, 33, 35, 58, 60, 62, 125, and 127 of SEQ ID NO:4.

In one embodiment, the variant VV H3L protein comprises amino acid substitution or deletion at one or more of the following amino acid residues: 14, 15, 16, 33, 34, 35, 38, 40, 44, 45, 52, 131, 132, 134, 135, 136, 137, 154, 155, 156, 161, 166, 167, 168, 195, 198, 199, 227, 250, 251, 252, 253, 254, 255, 256, 258, 262, 264, 266, 268, 272, 273, 275, and 277 of SEQ ID NO:170.

In one embodiment, the variant VV D8L protein comprises amino acid substitution or deletion at one or more of the following amino acid residues: 43, 44, 48, 53, 54, 55, 98, 108, 109, 144, 168, 177, 196, 199, 203, 207, 212, 218, 220, 222, and 227 of SEQ ID NO:172.

In one embodiment, the heterologous nucleic acid carried by the recombinant VV encodes a domain of a regulator of complement activation. Examples of regulator of complement activation include, but are not limited to, CD55, CD59, CD46, CD35, factor H, and C4-binding protein. In one embodiment, the heterologous nucleic acid encodes a CD55 polypeptide comprising the amino acid sequence of SEQ ID NO:7.

In another embodiment, the heterologous nucleic acid carried by the recombinant VV encodes a bi-specific polypeptide that binds to a first antigen on immune cells and a second antigen on tumor cells. In one embodiment, the first antigen on immune cells can be CD3, CD4, CDS, CD8, CD16, CD28, CD40, CD64, CD89, CD134, CD137, NKp46, or NKG2D. In one embodiment, the second antigen on tumor cells can be fibroblast activation protein (FAP), or tumor antigens on multiple myeloma.

In one embodiment, the bi-specific polypeptide is a bi-specific scFvs, the first antigen is human CD3e and the second antigen is human FAP. For example, this bi-specific polypeptide has the amino acid sequence of SEQ ID NO:8.

In another embodiment, the bi-specific polypeptide can target tumor antigens on multiple myeloma, e.g. B-cell maturation antigen (BCMA), CD19, CD38, SLAMF7, CD26, LIGHT/TNFSF14, integrin beta7, CD138, KIRs, EGFR, PD-1/PD-L1, TGIT, CD56, CS1, NKG2D, TACI, or CD44v6. In one embodiment, the bi-specific polypeptide is a bi-specific scFvs, the first antigen is human CD3e and the second antigen is human BCMA. For example, this bi-specific polypeptide has the amino acid sequence of SEQ ID NO:9.

In another embodiment, the heterologous nucleic acid carried by the recombinant VV encodes a fusion polypeptide comprising an immune checkpoint molecule. Examples of immune checkpoint molecule include, but are not limited to, PD-1, PD-L1, PD-L2, CD47, CXCR4, CSF1R, LAG-3, TIM-3, HHLA2, BTLA, CTLA-4, TIGIT, VISTA, B7-H4, CD160, 2B4, and CD73. In one embodiment, the heterologous nucleic acid carried by the recombinant VV encodes a fusion polypeptide comprising human PD-1 extracellular domain and a human IgG1 Fc domain, e.g., this fusion polypeptide has the amino acid sequence of SEQ ID NO:10.

In one embodiment, the recombinant vaccinia virus (VV) virion disclosed herein exhibits resistance to neutralizing antibodies compared to the resistance exhibited by wild type VV. In another embodiment, the recombinant vaccinia virus (VV) virion disclosed herein exhibits increased transduction of mammalian cells in the presence of anti-VV neutralizing antibodies compared to transduction of mammalian cells by wild type VV.

In another embodiment, there is provided a method of delivering a gene product to a subject (human or animal) in need thereof. The method includes administering to the subject an effective amount of the recombinant vaccinia virus (VV) virion disclosed herein, wherein the gene product is encoded by the heterologous nucleic acid carried by the recombinant VV virion.

In another embodiment, there is provided a pharmaceutical composition comprising the recombinant vaccinia virus (VV) virions disclosed herein and a pharmaceutically acceptable carrier. In another embodiment, there is provided a method of using such pharmaceutical compositions to treat cancer in a subject. In one embodiment, the pharmaceutical compositions can be administered to the subject intravenously, or through injection, inhalant, infusion, implantation, parenteral administration, enteral administration (e.g. through the gastrointestinal tract), or other systemic administration approach generally known in the art. In one embodiment, the subject is a human. Alternatively, the present invention may also be used in administration to and treatment of animal subjects.

In another embodiment, there is provided a library comprising one or more variant vaccinia virus (VV) virions, each of the variant VV virions comprises one or more variant VV protein. The variant VV protein comprises an amino acid sequence having at least one amino acid substitution or deletion relative to the amino acid sequence of a corresponding wild type VV protein. In one embodiment, the variant VV protein can be variant H3L protein, variant D8L protein, variant L1R protein, and/or variant A27L protein. In another embodiment, the variant VV protein comprises an amino acid sequence having at least one amino acid substitution or deletion relative to the amino acid sequence set forth in one of SEQ ID NOs:5, 6 or 174.

In another embodiment, there are provided variant vaccinia virus (VV) virions derived from the above library, the virions comprises a heterologous nucleic acid and one or more variant VV proteins, wherein at least one of the variant VV proteins comprises an amino acid sequence having at least one amino acid substitution or deletion relative to the amino acid sequence of a corresponding wild type VV protein. In one embodiment, the heterologous nucleic acid carried by such variant VV virions encodes a domain of a regulator of complement activation such as CD55, CD59, CD46, CD35, factor H, or C4-binding protein. For example, the heterologous nucleic acid encodes a CD55 protein that comprises the amino acid sequence of SEQ ID NO:7. In another embodiment, the heterologous nucleic acid encodes a bi-specific polypeptide that binds to a first antigen on immune cells and a second antigen on tumor cells. Examples of such first antigen and second antigen have been discussed above. In one embodiment, the bi-specific polypeptide is a bi-specific scFvs, the first antigen is human CD3e and the second antigen is human FAP, e.g. this bi-specific scFvs comprises the amino acid sequence of SEQ ID NO:8. In another embodiment, the bi-specific polypeptide is a bi-specific scFvs, the first antigen is human CD3e and the second antigen is human BCMA, e.g. this bi-specific scFvs comprises the amino acid sequence of SEQ ID NO:9. In yet another embodiment, the heterologous nucleic acid encodes a fusion polypeptide comprising an immune checkpoint molecule as discussed above. In one embodiment, the fusion polypeptide comprises human PD-1 extracellular domain and a human IgG1 Fc domain, the fusion polypeptide having the amino acid sequence of SEQ ID NO:10.

In one embodiment, the variant VV virions derived from the above library exhibit resistance to neutralizing antibodies compared to the resistance exhibited by wild type VV. In another embodiment, these variant VV virions exhibit increased transduction of mammalian cells in the presence of anti-VV neutralizing antibodies compared to transduction of mammalian cells by wild type VV.

In another embodiment, there is provided a method of using an effective amount of recombinant vaccinia virus (VV) virions derived from the above library to deliver a gene product to a subject (human or animal) in need thereof, wherein the gene product is encoded by a nucleic acid carried by those variant VV virions.

In another embodiment, there is provided a pharmaceutical composition comprising variant vaccinia virus (VV) virions derived from the above library and a pharmaceutically acceptable carrier. In another embodiment, there is provided a method of using such pharmaceutical composition to treat cancer in a subject. In one embodiment, the pharmaceutical composition can be administered to the subject intravenously, or through injection, inhalant, infusion, implantation, parenteral administration, enteral administration (e.g. through the gastrointestinal tract), or other systemic administration approach generally known in the art. In one embodiment, the subject is a human, but the technology may also be used in administration to and treatment of animal subjects.

In another embodiment, there is provided a recombinant vaccinia virus (VV) H3L protein that has at least about 60% amino acid sequence identity to one of SEQ ID NOs:1, 5 or 170. In another embodiment, there is provided a recombinant vaccinia virus D8L protein that has at least about 60% amino acid sequence identity to one of SEQ ID NOs:2, 6, 172 or 174. These recombinant H3L or D8L proteins could confer viral resistance to anti-VV neutralizing antibodies.

The invention being generally described, will be more readily understood by reference to the following examples which are included merely for purposes of illustration of certain aspects and embodiments of the present invention, and are not intended to limit the invention.

### EXAMPLE 1

### Materials and Methods

### Materials

pUC57-Amp A27L, pUC57-Amp L1R, pUC57-Amp D8L, pUC57-Amp H3L, (GENEWIZ). CV-1 cells (ATCC, cat. #CCL-70). vSC20 Vaccinia virus stock. GeneJuice Transfection Reagent (Millipore, cat. #2703870). DMEM media (GE Helathcare, cat. #SH30081.01), FBS (GE Healthcare, cat. #SH30070.03), DPBS (Sigma, cat. #8537). Dry ice/ethanol bath, 6-well tissue culture plates, 12×75-mm polystyrene tubes, disposable scraper or plunger from a 1 ml syringe, sterile 2-ml sterile microcentrifuge tubes.

Cell Preparation and Infection with Wild-Type Vaccinia Virus

CV-1 cells (2×10⁵/well) were seeded in wells of a 6-well tissue culture plate in complete DMEM medium and incubate to 50-80% confluency (37° C., 5% CO₂ overnight). An aliquot of parental virus was thawed and sonicated (30 sec) in ice-water several times to remove the clumps (cool on ice between each sonication). Virus was diluted in complete DMEM to 0.5×10⁵ pfu/ml. Medium was remove from confluent monolayer of cells and cells were infected with 0.5 ml diluted vaccinia virus (0.05 pfu/cell) and incubated 2 hrs at 37° C.

Transfection with pUC57-Amp Plasmid

For each well to be transfected, 100 μl serum-free medium was added into a sterile tube. Three μl GeneJuice was then added drop-wise directly to the serum-free medium and mixed thoroughly by vortexing and incubate at room temperature for 5 min. One μg of DNA was added to each tube and mixed by gentle pipetting (do not vortex) followed by incubation at room temperature for 5-15 min. Virus inoculum was then removed from monolayer of cells and washed twice with PBS. 0.5 mL of fresh complete DMEM medium was then added to the cells. The entire volume of GeneJuice/DNA mixture was then added drop-wise to cells in complete DMEM medium. The dish was gently rocked to ensure even distribution. Transfection mixture was removed after 4-8 hrs incubation and replaced with complete DMEM medium followed by incubation for 24-72 hrs at 37° C. (5% CO2). After 24-72 hours, the cells were dislodged from the wells and transferred to a 2-ml sterile microcentrifuge tube. The cell suspension was then lysed by performing three freeze-thaw cycles, each time by freezing in a dry ice/ethanol bath, thawing in a 37° C. water bath, and vortexing. The cell lysate was stored at −80° C. until needed

### Screening of Recombinant Virus Plaques

CV1 cells (5×10⁵/well) were seeded in a 6-well tissue culture plate in complete DMEM medium (2mL/well) and incubate to >90% confluency (37° C., 5% CO₂, 24 hrs). One hundred, 10, 1, or 0.1 μl of lysate were added to duplicate wells containing 1 ml complete DMEM medium and incubate 2 hrs at 37° C. The virus inoculum was then removed from the infected cells. 2 ml of complete DMEM medium containing 2.5% methylcellulose was added to each well with and incubated 2 days. Two days later, well-separated plaques were picked up by scraping and suction with a pipet tip. Fluorescent microscope was used to select GFP+ plaques that was transferred to a tube containing 0.5 ml complete DMEM medium. Each virus-containing tube was vortexed followed by three freeze-thaw cycles, each time by freezing in a dry ice/ethanol bath, thawing in a 37° C. water bath, and vortexing.

### Several Rounds of GFP+ Plaque Purification

Wells of a 6-well tissue culture plate were seeded with 5×10⁵ CV1 cells/well in complete DMEM medium (2mL/well). The cells were incubated to >90% confluency (37° C., 5% CO₂, 24 hrs). One 6-well plate is needed for each plaque isolate. One hundred, 10, 1, or 0.1 μl of lysate from each plaque were added to duplicate wells containing 1 ml complete DMEM medium, and incubated for 2 hrs. Medium was removed from the cell monolayers and overlay with complete DMEM containing 2.5% methylcellulose. The above steps were repeated for three or more rounds of plaque purification to ensure a clonally pure recombinant virus.

Single Plaque Purification Protocol

About 3-4 millions CV-1 cells were seeded and grown to 100% confluence in 24 well plate. The concentrated virus stock was diluted in 10-fold series dilutions with DMEM infection medium and added to each well. After 36-72 hour incubation, the wells that contain single plaque was marked and kept in the incubator until the whole well got infected, which takes about 4-5 days after initial infection. The infected cells were harvested and the recombination was confirmed by PCR assay. PCR conditions are listed below for each reaction.

TABLE 1

| PCR setup | (μL) |
|---|---|
| Nuclease-free water | 12 |
| 10XPCR Buffer | 2 |
| 50 mM MgCl | 0.6 |
| 10 mM dNTP Mix | 0.4 |
| Forward primer (5 μM) | 2 |
| Reverse primer (5 μM) | 2 |
| AccuStart Taq DNA | 0.08 |
| polymerase | |
| Template | 1 |
| Total | 20.08 |

TABLE 2

| | | | |
|---|---|---|---|
| Step | Temperature | Time | Note |

| | | | | | |
|---|---|---|---|---|---|
| 1 | 94° | C. | 1 | min | |
| 2 | 94° | C. | 20 | s | |
| 3 | 60° | C. | 30 | s | |
| 4 | 72° | C. | 30 | s | Go to step 2 |
| | | | | | for 34 cycles |

| | | | |
|---|---|---|---|
| 5 | 4° | C. | Hold |

### EXAMPLE 2

### Neutralizing Antibody (Nab) Epitope Determination on H3L—Peptide Arrays Sequence Analysis

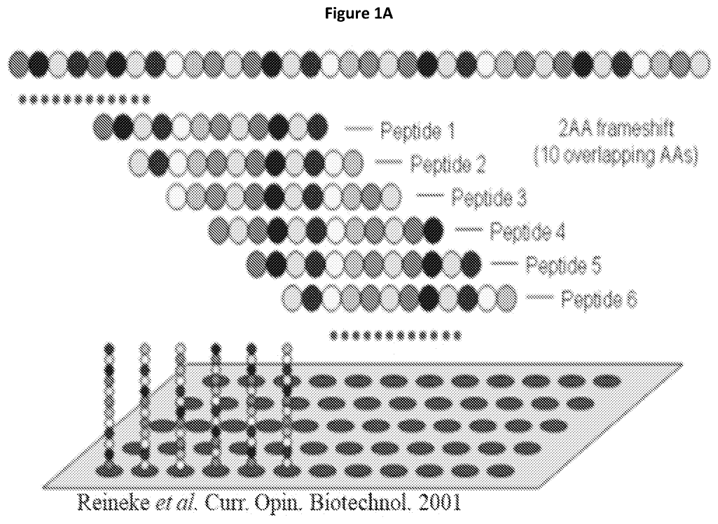

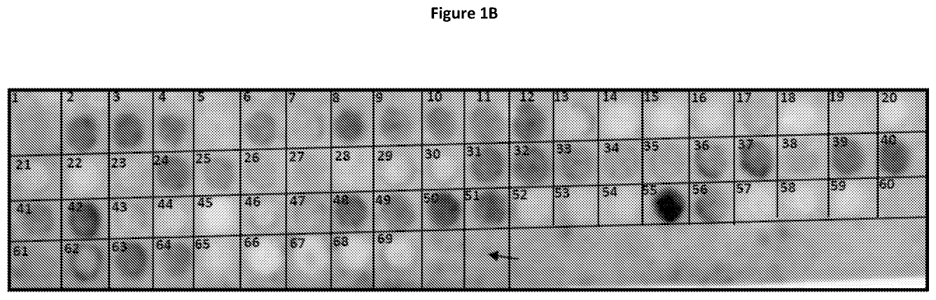

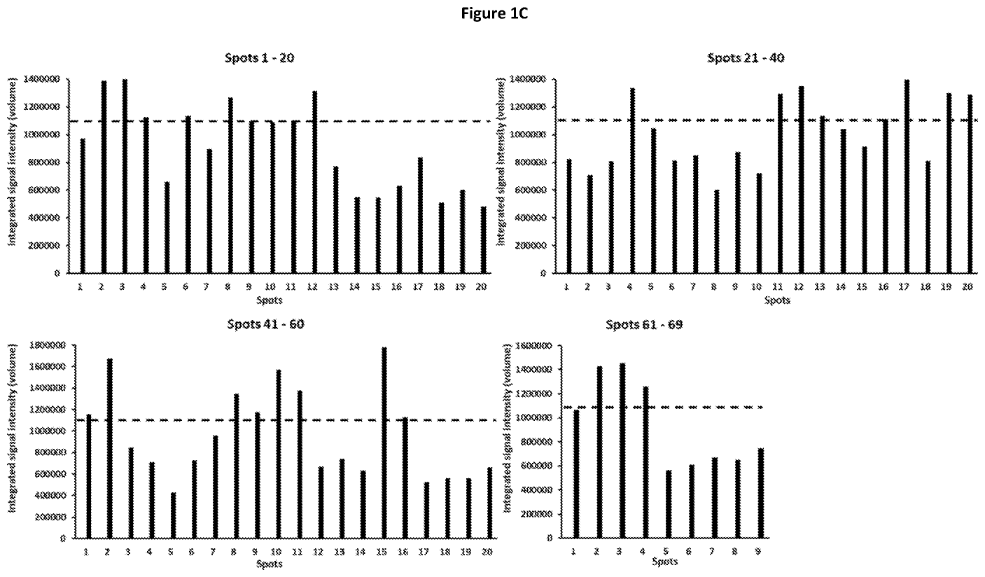

To identify possible regions on H3L that participate in the NAb interaction, peptide arrays encompassing full-length H3L were synthesized and screened for peptides that bound the anti-VV NAb. The array started at the N terminus of H3L and spanned the entire length of the protein sequence, with each successive spot containing 12 amino acids along the sequence shifted by 4 amino acids toward the C terminus, i.e., each spot in the array had an 8-residue overlap with the previous spot. Cellulose membrane containing synthesized H3L peptide array was then screened to identify peptides that bound to anti-VV polyclonal NAb (Abcam, ab35219). Briefly, the membrane was washed three times for 5 min in Millipore H₂O and blocked overnight at 4° C. with 5% (wt/vol) milk-PBS (MPBS). Four μg/mL NAb was incubated with the membrane in MPBS for 3 h at room temperature with gentle agitation. After incubation, membrane was washed six times for 5 min with 20 mL PBS supplemented with 1% Tween 20 (PBST). The peptide-bound NAb was detected by incubating the membrane with 2 μg/ml of rabbit horseradish peroxidase (HRP)-conjugated secondary Ab (Abcam, ab6721) in MPBS for 4 h at 4° C. with gentle agitation. The membrane was then washed three times for 5 min with PBST, incubated in 5 ml of the enhanced chemiluminescence (ECL) developing solution (Thermo Fisher, #32109). Peptides that are positive for binding appear as spots on the membranes (FIG. 1B). The signal was visualized, and the intensity of each spot was measured by a CCD camera (GE Healthcare, Amersham™ Imager 600). No oversaturation of the spots was detected and after integrating, the intensities of the spots were plotted (FIG. 1C). A signal of ≤110000 was considered background (determined by analysis of the membrane) and the spots showing a signal higher than 1100000 were considered to represent positive binding. Twenty six spots showed binding to ab35219 with higher than the cutoff intensity. To take into consideration that some positive signals could represent nonspecific binding, only those residues that were present in at least two spots that showed a binding intensity ≥1100000 were considered significant. In total 9 peptide sequences were identified positive for Ab binding (sequences appeared in multiple spots with positive binding signal, sequences with underlines shown below).

TABLE 3
Sequences of The Spots (And Their Corresponding
Positions) Synthesized Onto A Peptide Array

| | |
|---|---|
| 1 | MAAAKTPVIVVP (SEQ ID NO: 20) |
| 2 | KTPVIVVPVIDR (SEQ ID NO: 21) |
| 3 | IVVPVIDRLPSE (SEQ ID NO: 22) |
| 4 | VIDRLPSETFPN (SEQ ID NO: 23) |
| 5 | LPSETFPNVHEH (SEQ ID NO: 24) |
| 6 | TFPNVHEHINDQ (SEQ ID NO: 25) |
| 7 | VHEHINDQKFDD (SEQ ID NO: 26) |
| 8 | INDQKFDDVKDN (SEQ ID NO: 27) |
| 9 | KFDDVKDNEVMP (SEQ ID NO: 28) |
| 10 | VKDNEVMPEKRN (SEQ ID NO: 29) |
| 11 | EVMPEKRNVVVV (SEQ ID NO: 30) |
| 12 | EKRNVVVVKDDP (SEQ ID NO: 31) |
| 13 | VVVVKDDPDHYK (SEQ ID NO: 32) |
| 14 | KDDPDHYKDYAF (SEQ ID NO: 33) |
| 15 | DHYKDYAFIQWT (SEQ ID NO: 34) |
| 16 | DYAFIQWTGGNI (SEQ ID NO: 35) |
| 17 | IQWTGGNIRNDD (SEQ ID NO: 36) |
| 18 | GGNIRNDDKYTH (SEQ ID NO: 37) |
| 19 | RNDDKYTHFFSG (SEQ ID NO: 38) |
| 20 | KYTHFFSGFCNT (SEQ ID NO: 39) |
| 21 | FFSGFCNTMCTE (SEQ ID NO: 40) |
| 22 | FCNTMCTEETKR (SEQ ID NO: 41) |
| 23 | MCTEETKRNIAR (SEQ ID NO: 42) |
| 24 | ETKRNIARHLAL (SEQ ID NO: 43) |
| 25 | NIARHLALWDSN (SEQ ID NO: 44) |
| 26 | HLALWDSNFFTE (SEQ ID NO: 45) |
| 27 | WDSNFFTELENK (SEQ ID NO: 46) |
| 28 | FFTELENKKVEY (SEQ ID NO: 47) |
| 29 | LENKKVEYVVIV (SEQ ID NO: 48) |
| 30 | KVEYVVIVENDN (SEQ ID NO: 49) |
| 31 | VVIVENDNVIED (SEQ ID NO: 50) |
| 32 | ENDNVIEDITFL (SEQ ID NO: 51) |
| 33 | VIEDITFLRPVL (SEQ ID NO: 52) |
| 34 | ITFLRPVLKAMH (SEQ ID NO: 53) |
| 35 | RPVLKAMHDKKI (SEQ ID NO: 54) |
| 36 | KAMHDKKIDILQ (SEQ ID NO: 55) |
| 37 | DKKIDILQMREI (SEQ ID NO: 56) |
| 38 | DILQMREIITGN (SEQ ID NO: 57) |
| 39 | MREIITGNKVKT (SEQ ID NO: 58) |
| 40 | ITGNKVKTELVM (SEQ ID NO: 59) |
| 41 | KVKTELVMDKNH (SEQ ID NO: 60) |
| 42 | ELVMDKNHAIFT (SEQ ID NO: 61) |
| 43 | DKNHAIFTYTGG (SEQ ID NO: 62) |
| 44 | AIFTYTGGYDVS (SEQ ID NO: 63) |
| 45 | YTGGYDVSLSAY (SEQ ID NO: 64) |
| 46 | YDVSLSAYIIRV (SEQ ID NO: 65) |
| 47 | LSAYIIRVTTEL (SEQ ID NO: 66) |
| 48 | IIRVTTELNIVD (SEQ ID NO: 67) |
| 49 | TTELNIVDEIIK (SEQ ID NO: 68) |
| 50 | NIVDEIIKSGGL (SEQ ID NO: 69) |
| 51 | EIIKSGGLSSGF (SEQ ID NO: 70) |
| 52 | SGGLSSGFYFEI (SEQ ID NO: 71) |
| 53 | SSGFYFEIARIE (SEQ ID NO: 72) |
| 54 | YFEIARIENEMK (SEQ ID NO: 73) |
| 55 | ARIENEMKINRQ (SEQ ID NO: 74) |
| 56 | NEMKINRQILDN (SEQ ID NO: 75) |
| 57 | INRQILDNAAKY (SEQ ID NO: 76) |
| 58 | ILDNAAKYVEHD (SEQ ID NO: 77) |
| 59 | AAKYVEHDPRLV (SEQ ID NO: 78) |
| 60 | VEHDPRLVAEHR (SEQ ID NO: 79) |
| 61 | PRLVAEHRFENM (SEQ ID NO: 80) |
| 62 | AEHRFENMKPNF (SEQ ID NO: 81) |
| 63 | FENMKPNFWSRI (SEQ ID NO: 82) |
| 64 | KPNFWSRIGTAA (SEQ ID NO: 83) |
| 65 | WSRIGTAATKRY (SEQ ID NO: 84) |
| 66 | GTAATKRYPGVM (SEQ ID NO: 85) |
| 67 | TKRYPGVMYAFT (SEQ ID NO: 86) |
| 68 | PGVMYAFTTPLI (SEQ ID NO: 87) |
| 69 | YAFTTPLISFFG (SEQ ID NO: 88) |

### Sequences of the H3L Peptides (With Corresponding Residue Numbers) Identified by Peptide Array

PVIDRLP (aa 11-18) (SEQ ID NO:89), NDQKFDDVKDN (aa 30-40) (SEQ ID NO:90), PERKNVVVV (aa 44-52) (SEQ ID NO:91), NVIEDITFLR (aa 128-137) (SEQ ID NO:92), QMREI (aa 152-156) (SEQ ID NO:93), KVKTELVM (aa 161-168) (SEQ ID NO:94), NIVDEIIK (aa 197-204) (SEQ ID NO:95), KINRQI (aa 224-229) (SEQ ID NO:96), FENMKPNF (aa 249-265) (SEQ ID NO:97).

Ab-binding sites localized to the N-terminal domain (aa 11 to 52), the central (aa 128 to 168) and the C-terminal portions (aa 198 to 256) of H3L. Interestingly, the most C-terminal domain of the protein (aa 260 to 324) showed no binding to the Ab. This hydrophobic region of the H3L inserts into VV membrane post-translationally and would not be available for Ab binding in the context of the mature viral particle. The N-terminal domain is most likely involved in the binding of H3L to surface of cells, thus binding of the Ab to this region would interfere with the ability of the virus to infect the cells, supporting our array result of this region being involved in Ab binding. Additionally, an earlier study showed that H3L is a glycosyltransferase. Some viruses encode their own glycosyltransferases to aid in host immune response evasion. H3L binds the UDP-Glc via the D/ExD motif in its central domain and mutating this motif (aa 125 and 127, specifically) inhibited the binding. The peptide array showed a likely Ab binding site near the D/ExD motif (peptide NVIEDITFLR, aa 128-137 (SEQ ID NO:92)). Binding of the Ab in this region would interfere with the glycosyltransferase activity of the H3L, another possible mechanism of virus neutralization by the Ab.

### EXAMPLE 3

### NAb Epitope Determination of H3L—Alanine Scan of the Identified Peptides

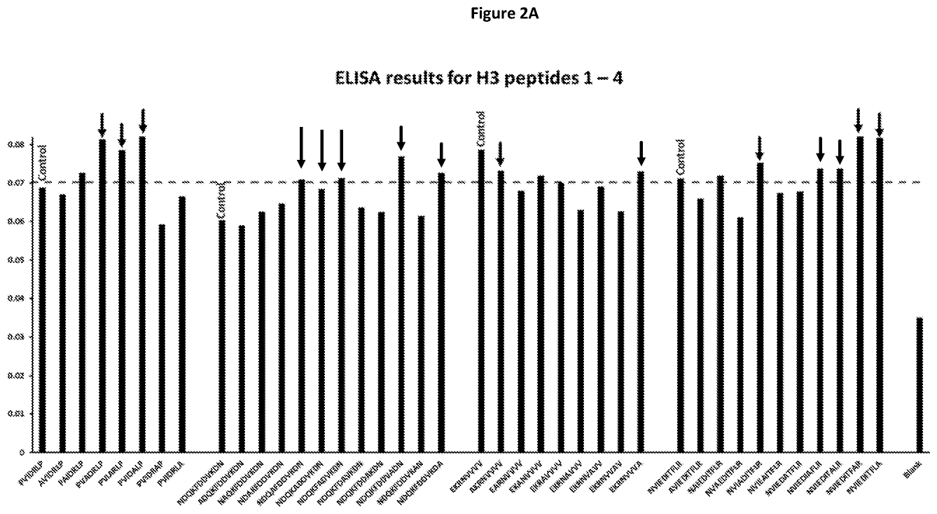

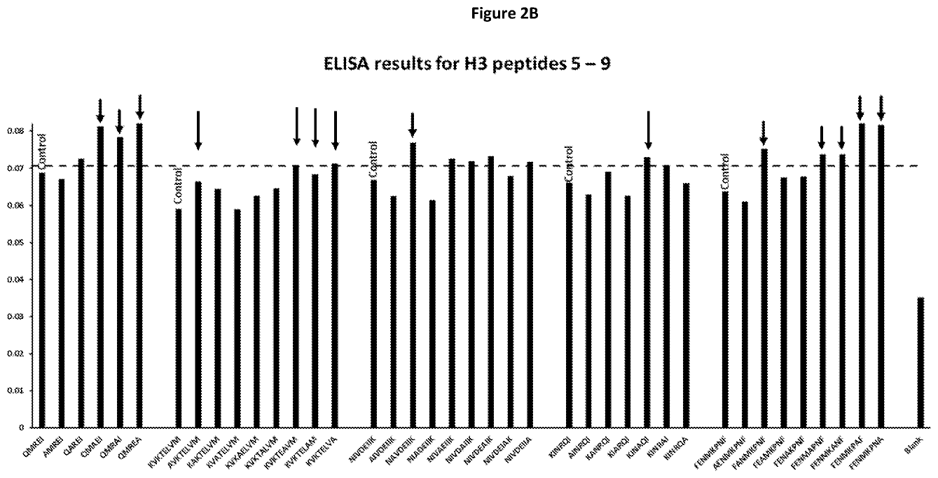

To further map the NAb epitopes and to elucidate the key residues on the H3L peptides identified by our peptide array study, a series of ELISAs were performed with the 9 identified peptides and their alanine-substituted variants (FIG. 2). Variants of the 9 peptides identified by peptide array were synthesized with alanine substitutions (GenScript USA Inc. NJ, USA).

TABLE 4
Total of 80 variant peptides were synthesized

| Peptide 1 | Peptide 2 | Peptide 3 |
|---|---|---|
| PVIDRLP | NDQKFDDVKDN | PEKRNVVVV |
| (SEQ ID NO: 89) | (SEQ ID NO: 90) | (SEQ ID NO: 91) |
| AVIDRLP (SEQ | ADQKFDDVKDN | AKRNVVVV |
| ID NO: 98) | (SEQ ID NO: 105) | (SEQ ID NO: 116) |
| PAIDRLP (SEQ | NAQKFDDVKDN | EARNVVVV |
| ID NO: 99) | (SEQ ID NO: 106) | (SEQ ID NO: 117) |
| PVADRLP (SEQ | NDAKFDDVKDN | EKANVVVV |
| ID NO: 100) | (SEQ ID NO: 107) | (SEQ ID NO: 118) |
| PVIARLP (SEQ | NDQAFDDVKDN | EKRAVVVV |
| ID NO: 101) | (SEQ ID NO: 108) | (SEQ ID NO: 119) |
| PVIDALP (SEQ | NDQKADDVKDN | EKRNAVVV |
| ID NO: 102) | (SEQ ID NO: 109) | (SEQ ID NO: 120) |
| PVIDRAP (SEQ | NDQKFADVKDN | EKRNVAVV |
| ID NO: 103) | (SEQ ID NO: 110) | (SEQ ID NO: 121) |
| PVIDRLA (SEQ | NDQKFDAVKDN | EKRNVVAV |
| ID NO: 104) | (SEQ ID NO: 111) | (SEQ ID NO: 122) |
| | NDQKFDDAKDN | EKRNVVVA |
| | (SEQ ID NO: 112) | (SEQ ID NO: 123) |
| | NDQKFDDVADN | |
| | (SEQ ID NO: 113) | |
| | NDQKFDDVKAN | |
| | (SEQ ID NO: 114) | |
| | NDQKFDDVKDA | |
| | (SEQ ID NO: 115) | |
| Peptide 4 | Peptide 5 | Peptide 6 |
| NVIEDITFLR | QMREI | KVKTELVM (SEQ |
| (SEQ ID NO: 92) | (SEQ ID NO: 93) | ID NO: 94) |
| AVIEDITFLR (SEQ | AMREI (SEQ ID | AVKTELVM (SEQ ID |
| ID NO: 124) | NO: 134) | NO: 139) |
| NAIEDITFLR (SEQ | QAREI (SEQ ID | KAKTELVM (SEQ ID |
| ID NO: 125) | NO: 135) | NO: 140) |
| NVAEDITFLR (SEQ | QMAEI (SEQ ID | KVATELVM (SEQ ID |
| ID NO: 126) | NO: 136) | NO: 141) |
| NVIADITFLR (SEQ | QMRAI (SEQ ID | KVKAELVM (SEQ ID |
| ID NO: 127) | NO: 137) | NO: 142) |
| NVIEAITFLR (SEQ | QMREA (SEQ ID | KVKTALVM (SEQ ID |
| ID NO: 128) | NO: 138) | NO: 143) |
| NVIEDATFLR (SEQ | | KVKTEAVM (SEQ ID |
| ID NO: 129) | | NO: 144) |
| NVIEDIAFLR (SEQ | | KVKTELAM (SEQ ID |
| ID NO: 130) | | NO: 145) |
| NVIEDITALR (SEQ | | KVKTELVA (SEQ ID |
| ID NO: 131) | | NO: 146) |
| NVIEDITFAR (SEQ | | |
| ID NO: 132) | | |
| NVIEDITFLA (SEQ | | |
| ID NO: 133) | | |
| Peptide 7 | Peptide 8 | Peptide 9 |
| NIVDEIIK (SEQ ID | KINRQI (SEQ ID | FENMKPNF (SEQ |
| NO: 95) | NO: 96) | ID NO: 97) |
| AIVDEIIK (SEQ ID | AINRQI (SEQ ID | AENMKPNF (SEQ ID |
| NO: 147) | NO: 155) | NO: 161) |
| NAVDEIIK (SEQ ID | KANRQI (SEQ ID | FANMKPNF (SEQ ID |
| NO: 148) | NO: 156) | NO: 162) |
| NIADEIIK (SEQ ID | KIARQI (SEQ ID | FEAMKPNF (SEQ ID |
| NO: 149) | NO: 157) | NO: 163) |
| NIVAEIIK (SEQ ID | KINAQI (SEQ ID | FENAKPNF (SEQ ID |
| NO: 150) | NO: 158) | NO: 164) |
| NIVDAIIK (SEQ ID | KINRAI (SEQ ID | FENMAPNF (SEQ ID |
| NO: 151) | NO: 159) | NO: 165) |
| NIVDEAIK (SEQ ID | KINRQA (SEQ ID | FENMKANF (SEQ ID |
| NO: 152) | NO: 160) | NO: 166) |
| NIVDEIAK (SEQ ID | | FENMKPAF (SEQ ID |
| NO: 153) | | NO: 167) |
| NIVDEIIA (SEQ ID | | FENMKPNA (SEQ ID |
| NO: 154) | | NO: 168) |

The native peptides (non-mutated, shown above in bold, SEQ ID Nos:89-97) were tagged with biotin (N-Terminal). 96-well Pierce™ NeutrAvidin coated plates (Thermo Fisher, 15507) were rinsed with PBST and incubated overnight at 4° C. in the MPBS (blocking buffer, 100 μL/well). Blocking buffer was discarded, and 100 μL of biotinylated peptides was added to the plate at 200 ng/mL and incubated for 90 min at 4° C. Simultaneously, anti-VV rabbit polyclonal NAb (Abcam, ab35219) was incubated with variant peptides. We used 30 μL/well of Ab at 800 ng/mL and incubated it with 30 μL/well of alanine-modified peptides at 100 μg/mL for 90 min at 4° C. After washing the plates with PBST, 50 μL of the Ab/alanine peptide mix was added to plate-bound peptides (in duplicate wells) and incubated for 60 min at 4° C. Plates were washed with PBST six times, and 100 μL/well of anti-rabbit horseradish peroxidase (HRP)-conjugated secondary Ab (Abcam, ab6721) diluted 1:1000 in MPBS was added. The plates were then incubated for 90 min at 4° C., washed with PBST four times and developed using 3,3′,5,5′,-Tetramethylbenzidine (TMB) (Sigma, T0440-100ML). The OD at 650 nm was read on Perkin Elmer Multimode Plate Reader (Corning). The intensity of each signal was measured and plotted using Kaleido™ 1.2 software. For each set of mutant peptides, a signal higher that the native control for that set was considered positive (FIG. 2). Control peptide for set 3 peptides (EKRNVVVV (SEQ ID NO:169)) showed a signal higher than the rest of the peptides in the set with only two other peptides in this set showing a signal above 0.07. The scan identified a total of 29 residues positive for Ab binding: I14, D15, R16, K33, F34, D35, K38, N40, E45, V52, E131, T134, F135, L136, R137, R154, E155, I156, K161, L166, V167, M168, I198, R227, E250, K253, P254, N255, and F256 (FIG. 2). The peptide arrays involve linear peptides and therefore may not represent the physiological confirmations of the residues in the context of the 3D protein structure. To analyze each identified residue in the context of the full-length H3L protein we mapped them onto the previously determined crystal structure of H3L. All but two residues (N40 and F135) mapped to the surface of the protein and therefore would potentially be available for interaction with the Abs. N40 and F135 mapped on the inside folds of the protein and therefore would be unlikely to interact with the Abs. An additional residue P44 was identified by a separate experiment (see below) and therefore was also included in our design. Lastly, the alanine scan identified 8 additional residues that showed a signal lower than the cutoff but higher than their respective controls, suggesting that they may also play a role in the Ab binding: K33, F34, D35, K161, L166, V167, and R227 (see FIG. 2).

In one embodiment, a mutant H3L protein comprises the following mutations: 114A, D15A, R16A, K33A, F34A, D35A, K38A, N40A, E45A, V52A, E131A, T134A, F135A, L136A, R137A, R154A, E155A, I156A, K161A, L166A, V167A, M168A, I198A, R227A, E250A, K253A, P254A, N255A, and F256A. An example of mutant H3L amino acid sequence is shown in SEQ ID NO:1.

### EXAMPLE 4

### Homologous Recombination to Introduce Modified H3L, D8L, L1R, and A27L Genes Into the VV Genome

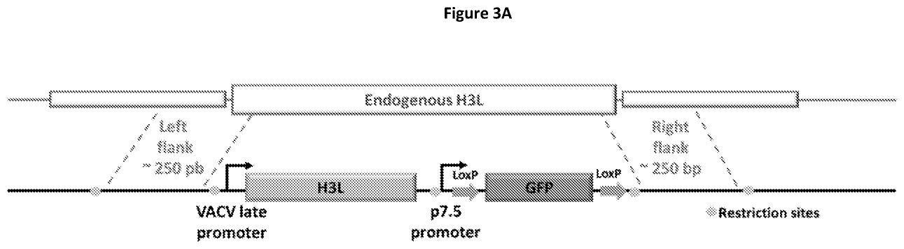

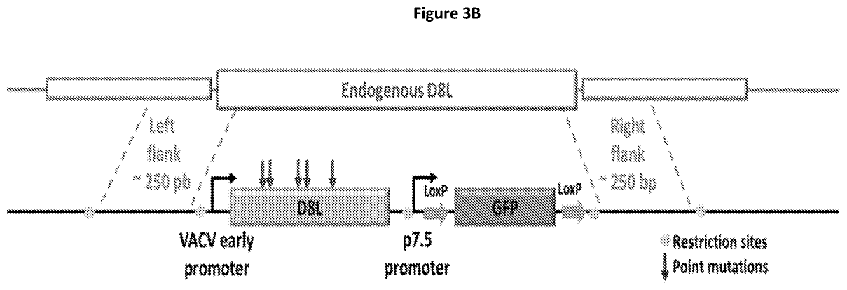

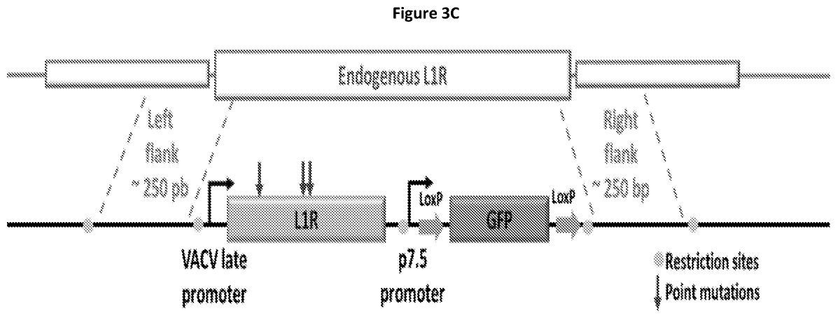

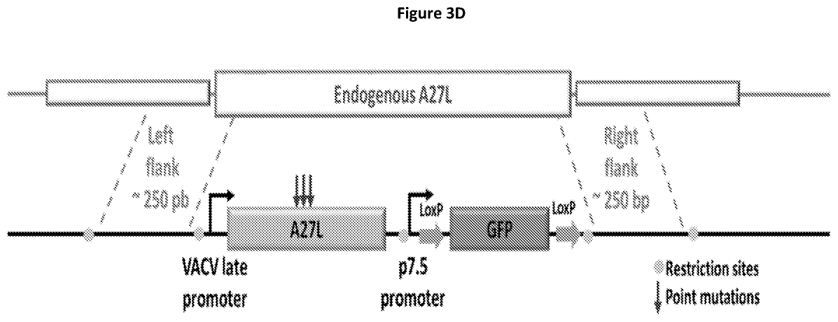

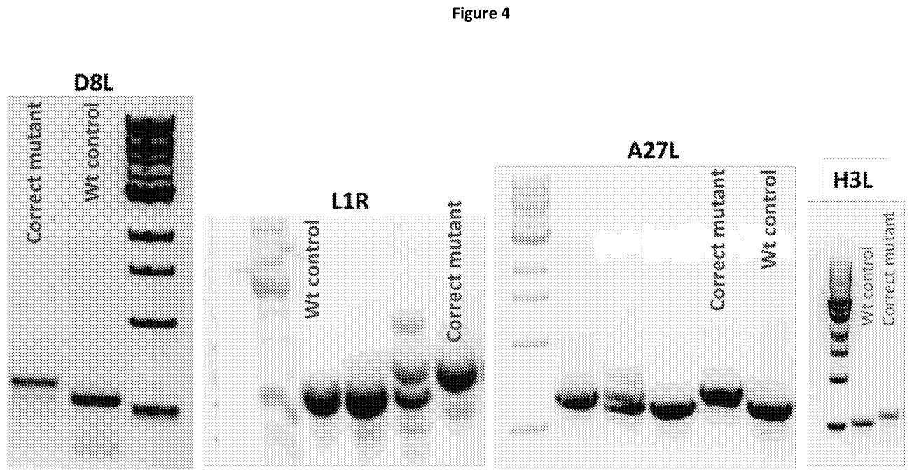

For each modified protein a DNA fragment containing the proteins' native promoter, ORF (with mutations in place), and approximately ˜250-bp flanking regions for homologous recombination into the appropriate gene in the VV genome was synthesized by GENEWIZ and cloned into the pUC57-Amp plasmid. For all four constructs a green fluorescent protein (GFP) expression cassette under the control of the VV p7.5 promoter and flanked by LoxP sites was inserted immediately downstream of the stop codon before the right flank sequence (FIG. 3). The fluorescence marker expressed from the GFP cassette was used to screen for clones that had undergone homologous recombination and GFP was removed using the LoxP sites. The pUC57-Amp plasmids were transfected into the CV-1 cells and allowed to recombine with the VV genome. The fluorescence marker expressed from the GFP cassette was used to screen for clones that had undergone homologous recombination (HR) and GFP was removed using the LoxP sites. The correct gene insertion into the VV genome was verified by PCR.The plasmids were transfected into the CV-1 cells infected with the VV one at a time, starting with the L1R plasmid, following by A27L, D8L, and finally H3L. With the addition of each plasmid rounds of screening and purification were performed, followed by PCR and sequencing to make sure that the correct mutations were present. GFP was removed before the recombination with the next plasmid. The final variant contains modifications in all four proteins.

Nucleotide substitutions in a synthesized H3L construct result in the following amino acid mutations: I14A, D15A, R16A, K38A, P44A, E45A, V52A, E131A, T134A, L136A, R137A, R154A, E155A, I156A, M168A, I198A, E250A, K253A, P254A, N255A, and F256A. The mutant H3L amino acid sequence is shown in SEQ ID NO:11. Nucleotide sequences for such mutated H3L gene, containing left flank region, promoter region, p7.5 promoter, LoxP, GFP, LoxP, and right flank regions are shown in SEQ ID NO:12.

Nucleotide substitutions in a synthesized D8L construct result in the following amino acid mutations: R44A, K48A, K98A, K108A, K117A, and R220A. The mutant D8L amino acid sequence is shown in SEQ ID NO:2. Nucleotide sequences for such mutated D8L gene, containing left flank region, promoter region, p7.5 promoter, LoxP, GFP, LoxP, and right flank regions are shown in SEQ ID NO:13.

Nucleotide substitutions in a synthesized A27L construct result in the following amino acid mutations: K27A, A30D, R32A, E33A, A34D, I35A, V36A, K37A, D39A, E40A, R107A, P108A, and Y109A. The mutant A27L amino acid sequence is shown in SEQ ID NO:3. Nucleotide sequences for such mutated A27L gene, containing left flank region, promoter region, p7.5 promoter, LoxP, GFP, LoxP, and right flank regions are shown in SEQ ID NO:14.

Nucleotide substitutions in a synthesized L1R construct result in the following amino acid mutations: E25A, N27A, Q31A, T32A, K33A, D35A, S58A, D60A, D62A, K125A, and K127A. The mutant L1R amino acid sequence is shown in SEQ ID NO:4. Nucleotide sequences for such mutated L1R gene, containing left flank region, promoter region, p7.5 promoter, LoxP, GFP, LoxP, and right flank regions are shown in SEQ ID NO:15.

### EXAMPLE 5

### In Vitro Neutralization Assays With Anti-VV Polyclonal Antibodies

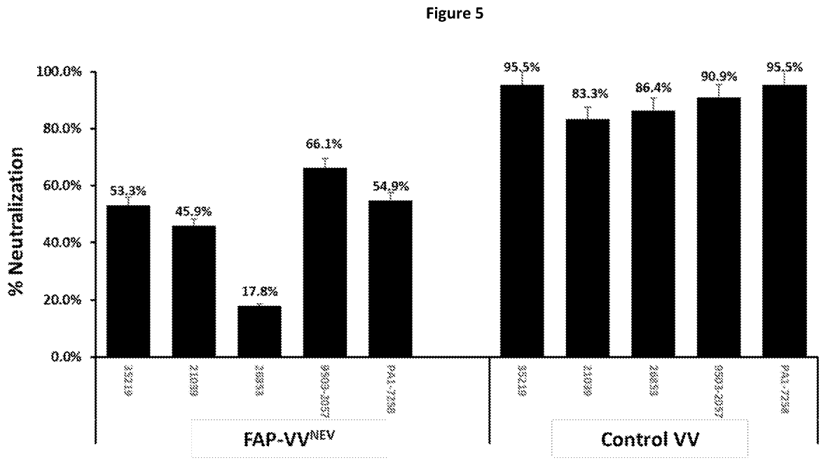

The ability of the anti-VV polyclonal Abs to neutralize the escape variants was investigated. A panel of anti-VV Abs consisting of ab35219 (Abcam), ab21039 (Abcam), ab26853 (Abcam), 9503-2057 (Bio-Rad), and PA1-7258 (Invitrogen) was used to test for neutralization escape in vitro. Rabbit polyclonal IgG ab37415 (Abcam) served as a control. CV-1 cells were seeded into 12-well plates and used within 2 days of reaching confluence. Forty μg/mL of Ab was preincubated with either the escape variant or the control VV at 1×10³ pfu/sample for 1 hr at 37° C. in the presence of 2% of sterile baby rabbit complement. The mixture was then added to the CV-1 cells and allowed to adhere for 2 hrs at 37° C./5% CO₂ in 300 μL of serum free media. After 2 hrs, the inoculum was removed and 1mL of complete DMEM medium was added to the cells. The cells were then incubated at 37° C./5% CO₂. After 48 hrs cells were fixed and stained with 1% crystal violet/20% EtOH solution for 20 min at room temperature and plaques were counted. All five Abs reduced the control VV plaque numbers dramatically, showing a strong neutralizing ability (FIG. 5). On average 83.3-95.5% of the control VV virus was neutralized across the panel. In contrast, the L1R+A27L+D8L+H3 escape variant showed a significantly lower neutralization by the Abs, with an average of 17.8-66.2% neutralization. Interestingly ab26853 neutralized 78% of control VV but almost completely failed to neutralize NEV variant (see FIG. 5). Based on these results, it is concluded that the escape variants disclosed herein can efficiently escape neutralization by anti-VV Abs in vitro.

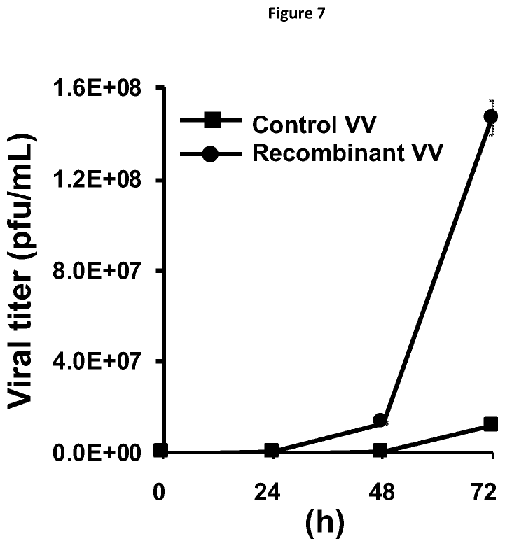

Recombinant virus replication assay was performed (FIG. 7). In a 24-well plate CV-1 cells were infected with duplicates of VV control, VVNEV, and VVEM at MOI=0.05. Prior to infection virus was preincubated with Ab 9503-2057 (40 μg/mL) for 1 hr at 37° C. Samples were collected at 24, 48, and 72 hrs and titers were determined for each time point. The recombinant virus was significantly more efficient in replicating in the presence of Ab, compared to the control Ab, which was almost entirely inactivated.

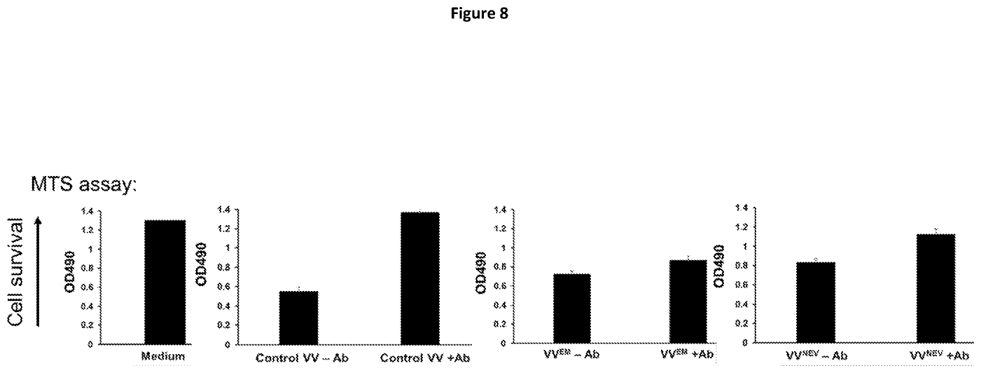

Anti-tumor efficiency of the recombinant virus was evaluated (FIG. 8). The recombinant virus and the control VV were preincubated with Ab 9503-2057 (see above) and used to infect transformed cells at MOI=1. Cells were incubated for 48 hrs and cell viability was measured by MTS assay (colorimetric assessment of cell metabolic activity). Briefly, cells collected at 48 hrs were washed once with PBST and resuspended at 1×105 cells/mL in complete DMEM. One hundred μL of each cell suspension was added to a 96-well (in triplicates). Twenty μl of CellTiter 96® AQueous One Solution Reagent (Promega, G358C) was added into each well of the 96-well assay plate containing the samples in 100 μl of culture medium. The plate was incubated at 37° C. for 2 hrs (5% CO2). To measure the amount of soluble formazan produced by cellular reduction of MTS, the absorbance in each well was recorded at 490 nm using a 96-well plate reader. In the presence of the Ab, the recombinant virus was able to efficiently kill the cells.

### EXAMPLE 6

### Isolation of Neutralization Escape Mutant (VV^{EM})

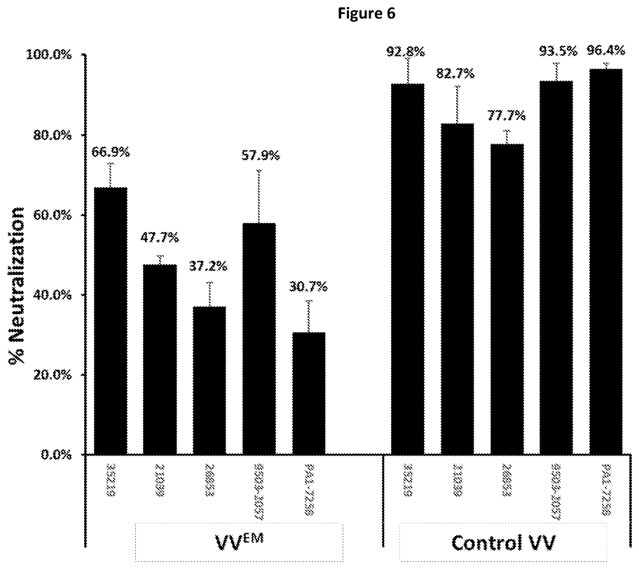

To identify any additional key NAb epitope residues, VV mutants that resisted the neutralization by ab35219 and ab21039 were selected. Briefly, a stock of mutant VV was prepared from CV-1 cells that were infected with the Western Reserve strain of VV in the presence of ethyl methanesulfonate (EMS) to induce transition mutations in viral DNA. Polyclonal anti-VV ab35219 and ab21039 were then used to neutralize the mutated virus. EMS was present in the culture medium at 500 μg/mL. The mutant viral stock was incubated with the mixture of two polyclonal Abs at 50 μg/ml each (100 μg/ml total conc.) for 1 hr, and then used to infect the CV-1 cells plated in the 12-well plates. After 2 hrs the inoculum was removed and fresh complete DMEM was added to the cells. Cells were then incubated at 37° C., 5% CO₂ for 48 hrs. During the first round of infection, the titer of the mutant virus was significantly reduced by the Abs. After a multiple rounds of infections with constant Ab concentration and with the increasingly more purified virus than the previous round, the passaged viral stock was no longer significantly neutralized by the Abs. A clone of the escape mutant (VV^{EM}) was plaque purified and showed a significant escape of neutralization by a panel of five anti-VV Abs described above (FIG. 6). Whereas on average 77.7-96.4% of the control VV virus was neutralized across the panel, VV^{EM} showed an average of 30.7-66.9% neutralization by the Abs, significantly lower than the control. Viral DNA from pure virus was isolated and PCR was used to amplify the A27L, L1R, H3L, and D8L genes, the major Ab antigens of the VV. PCR products were sequenced and showed presence of the mutations in the genes encoding A27L, D8L, and H3L. D8L coding sequence contains the following mutations: V43F/L, R44W, G55W, A144T, T168S, S177Y, F199Y, L203S, P212T, N218C, P222L, and D227G. The A27L coding sequence showed two mutations at residues 135 and D39 that were previously determined to be involved in the NAb interaction with A27L and were included in our A27L plasmid design. The H3L sequence showed an amino acid substitution at residue P44, a residue immediately adjacent to the E45 residue identified by the peptide array as part of the Ab-binding peptide (peptide 3; FIG. 2A) and thus was also included in the H3L recombinant plasmid design. Other mutations identified in the H3 gene are: E250G, N255W (these two residues were also identified by the alanine scan), S258F, T262P, A264T, T265V, K266I, Y268C, M272K, Y273N, F275N, and T277A. All of these mutations are clustered in the flexible C-terminal region of the protein. SEQ ID NO:5 shows a mutant H3L amino acid sequence. SEQ ID NO:6 or SEQ ID NO:174 shows a mutant D8L amino acid sequence. Both SEQ ID NOs:6 and 174 were disclosed in parent application U.S. Provisional Patent Application No. 62/749,102 as SEQ ID NO:7.

### EXAMPLE 7

### Homologous Recombination to Introduce Modified H3L, D8L, L1R, and A27L Genes Into the VV Genome

A new recombinant VV was made to incorporate the mutations that were identified as above. In addition, structural analysis of the proteins also identified additional residues that were not identified by either the peptide arrays or the EM sequencing but were adjacent to the residues that were identified and could potentially play a role in Ab interactions. Those residues were also included in the design. For each modified protein a DNA fragment containing the proteins' native promoter, ORF (with mutations in place), and approximately 250-bp flanking regions for homologous recombination into the appropriate gene in the VV genome was synthesized by GENEWIZ and cloned into the pUC57-Amp plasmid. For all four constructs a green fluorescent protein (GFP) expression cassette under the control of the VV p7.5 promoter and flanked by LoxP sites was inserted immediately downstream of the stop codon before the right flank sequence (FIG. 3). The fluorescence marker expressed from the GFP cassette was used to screen for clones that had undergone homologous recombination and GFP was removed using the LoxP sites. The pUC57-Amp plasmids were transfected into the CV-1 cells and allowed to recombine with the VV genome. The fluorescence marker expressed from the GFP cassette was used to screen for clones that had undergone homologous recombination (HR) and GFP was removed using the LoxP sites. The correct gene insertion into the VV genome was verified by PCR.The plasmids were transfected into the CV-1 cells infected with the VV one at a time, starting with the L1R plasmid, following by A27L, D8L, and finally H3L. With the addition of each plasmid rounds of screening and purification were performed, followed by PCR and sequencing to make sure that the correct mutations were present. GFP was removed before the recombination with the next plasmid. The final variant contains modifications in all four proteins.

Nucleotide substitutions in a synthesized H3L construct result in the following amino acid mutations: I14A, D15A, R16A, K33A, F34A, D35A, K38A, N40A, P44A, E45A, V52A, E131A, D132A, T134A, F135A, L136A, R137A, R154A, E155A, I156A, K161A, L166A, V167A, M168A, E195A, I198A, V199A, R227A, E250A, N251A, M252A, K253A, P254A, N255A, F256A, S258A, T262P, A264T, K266I, Y268C, M272K, Y273N, F275N, and T277A. The mutant H3L amino acid sequence is shown in SEQ ID NO:170. Nucleotide sequences for such mutated H3L gene, containing left flank region, promoter region, p7.5 promoter, LoxP, GFP, LoxP, and right flank regions are shown in SEQ ID NO:171.

Nucleotide substitutions in a synthesized D8L construct result in the following amino acid mutations: V43A, R44A, K48A, S53A, G54A, G55A, K98A, K108A, K109A, A144G, T168A, S177A, L196A, F199A, L203A, N207A, P212A, N218A, R220A, P222A, and D227A. The mutant D8L amino acid sequence is shown in SEQ ID NO:172. Nucleotide sequences for such mutated D8L gene, containing left flank region, promoter region, p7.5 promoter, LoxP, GFP, LoxP, and right flank regions are shown in SEQ ID NO:173.

Nucleotide substitutions in a synthesized A27L construct result in the following amino acid mutations: K27A, A30D, R32A, E33A, A34D, I35A, V36A, K37A, D39A, E40A, R107A, P108A, and Y109A. The mutant A27L amino acid sequence is shown in SEQ ID NO:3. Nucleotide sequences for such mutated A27L gene, containing left flank region, promoter region, p7.5 promoter, LoxP, GFP, LoxP, and right flank regions are shown in SEQ ID NO:14.

Nucleotide substitutions in a synthesized L1R construct result in the following amino acid mutations: E25A, N27A, Q31A, T32A, K33A, D35A, S58A, D60A, D62A, K125A, and K127A. The mutant L1R amino acid sequence is shown in SEQ ID NO:4. Nucleotide sequences for such mutated L1R gene, containing left flank region, promoter region, p7.5 promoter, LoxP, GFP, LoxP, and right flank regions are shown in SEQ ID NO:15.

### EXAMPLE 8

### In Vitro Neutralization Assays with Anti-VV Polyclonal Antibodies

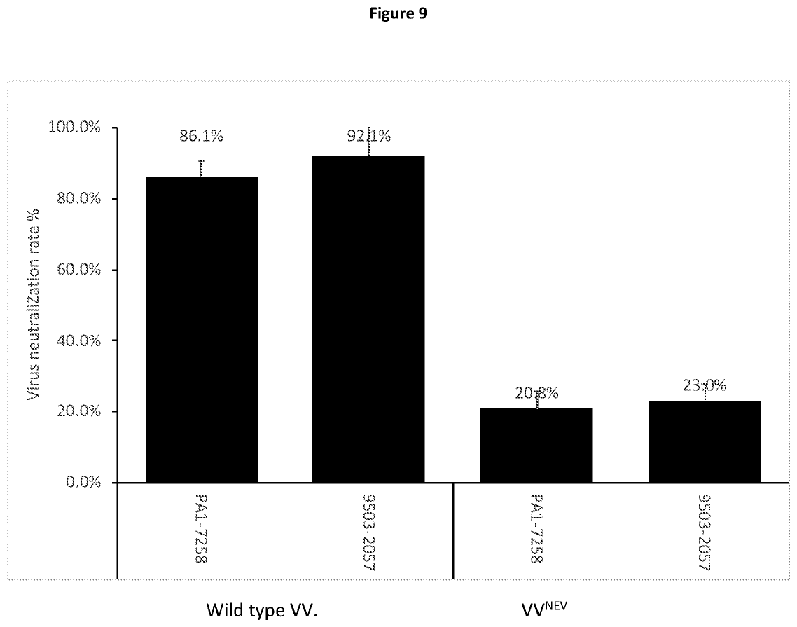

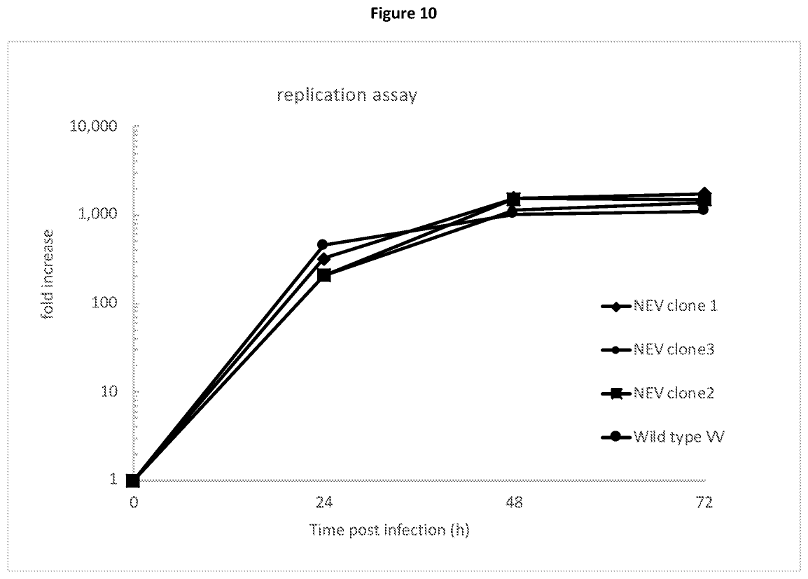

The ability of the anti-VV polyclonal Abs to neutralize the escape variants was investigated. Anti-VV Abs 9503-2057 (Bio-Rad) and PA1-7258 (Invitrogen) were used to test for neutralization escape in vitro. Rabbit polyclonal IgG ab37415 (Abcam) served as a control. CV-1 cells were seeded into 12-well plates and used within 2 days of reaching confluence. Forty μg/mL of Ab was preincubated with either the escape variant or the control VV at 1×10³ pfu/sample for 1 hr at 37° C. in the presence of 2% of sterile baby rabbit complement. The mixture was then added to the CV-1 cells and allowed to adhere for 2 hrs at 37° C./5% CO₂ in 300 μL of serum free media. After 2 hrs, the inoculum was removed and 1mL of complete DMEM medium was added to the cells. The cells were then incubated at 37° C./5% CO₂. After 48 hrs cells were fixed and stained with 1% crystal violet/20% EtOH solution for 20 min at room temperature and plaques were counted. NAbs reduced the control VV plaque numbers dramatically, showing a strong neutralizing ability (FIG. 9). On average 86.1-92.1% of the control VV virus was neutralized across the panel. In contrast, the escape variant showed a significantly lower neutralization by the Abs, with an average of 20.8-23% neutralization. Based on these results, it is concluded that the escape variants disclosed herein can efficiently escape neutralization by anti-VV Abs in vitro. The replication of the escape variant (3 single virus clones) and wild type VV were also compared in the absence of neutralization antibodies, the results suggested escape variants have similar replication capability compared to wild type virus, indicating that the mutation doeesn't impair the virus's entry and replication ability (FIG. 10).

### EXAMPLE 9

### Construction of VV Expressing CD55

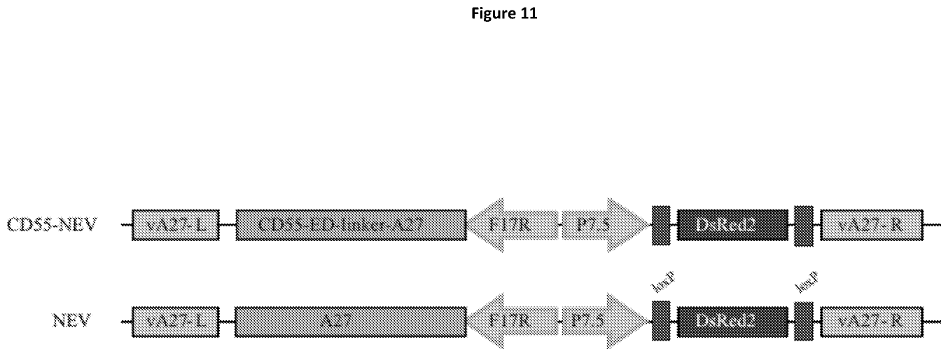

The oncolytic vaccinia virus (VV) construct CD55-NEV was generated to human CD55 extracellular domain. Human CD55 extracellular domain fused to VV A27 were optimized and synthesized and cloned into a pMS shuttle plasmid (FIG. 11). Vaccinia viruses (Western Reserve strain) expressing CD55-A27 were generated by recombination of a version of pMS shuttle plasmid into the TK gene of the WR vaccinia virus (WR VV) or NEV. The inserted CD55 and A27 was expressed under the transcriptional control of the original A27 promoter. To construct the recombinant virus CD55-NEV, the shuttle vectors pMS were transfected into CV-1 or 293 cells. Cells were then infected with WR VV or NEV at a multiplicity of infection (MOI) of 0.1. After three rounds of plaque selection and amplification to confirm the expression of CD55, one of the corresponding clones was selected for amplification and purification.

In one embodiment, an amino acid sequence comprising the CD55-A27 fusion is shown in SEQ ID NO:7. An example of an optimized nucleotide sequence for CD55-A27, containing signal peptide, CD55, A27 and linker sequence is shown in SEQ ID NO:16.

### EXAMPLE 10

### In Vitro Neutralization Assays with Complement or Complement/Anti-VV Polyclonal Antibodies

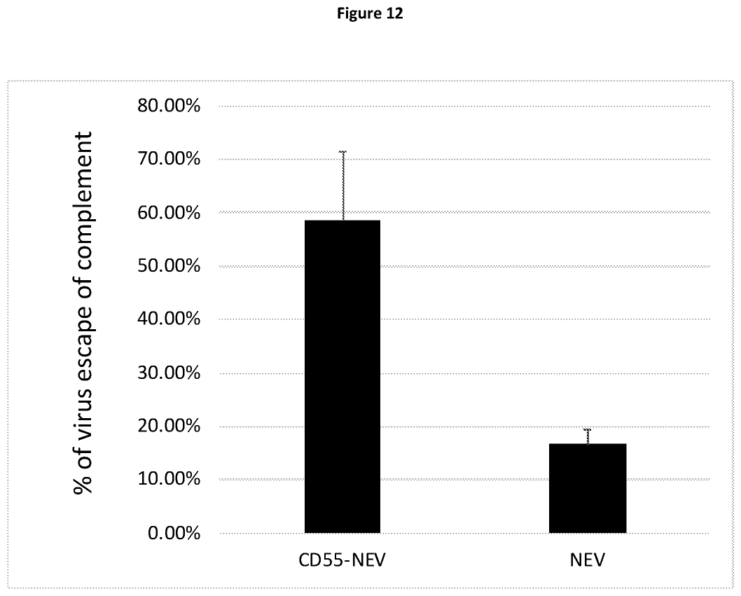

The ability of CD55-VV to escape complement-mediated neutralization was first investigated. To do this, CV-1 cells were seeded into 12-well plates and used within 2 days of reaching confluence. CD55-NEV or NEV control at 1×10³ pfu/sample were added to the CV-1 cells at 37° C./5% CO₂ in 300 μL of media in the presence of 1:10 human complement. Heat activated complement were used as control to calculate the escape rate. After 48 hrs, cells were fixed and stained with 1% crystal violet/20% EtOH solution for 20 min at room temperature and plaques were counted. CD55-NEV escaped complement-mediated neutralization more effectively than NEV (FIG. 12). Around 59% of the CD55-NEV escaped complement-mediated neutralization, while only around 18% of NEV escaped complement-mediated neutralization.

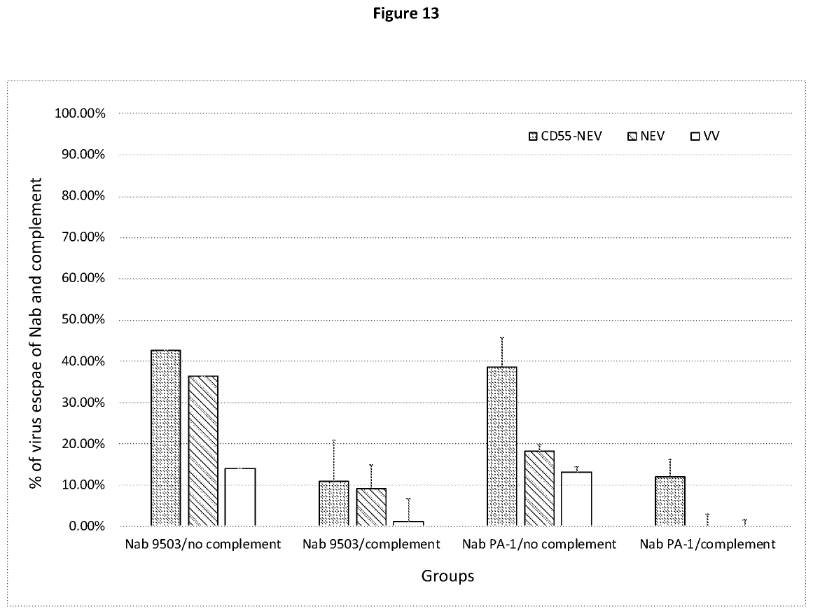

The ability of CD55-NEV to escape the neutralization of complement with anti-VV polyclonal Abs was further investigated. Two anti-VV Abs, 9503-2057 (Bio-Rad) and PA1-7258 (Invitrogen), were used to test for neutralization escape in vitro. CV-1 cells were seeded into 12-well plates and used within 2 days of reaching confluence. Forty μg/mL of Ab was preincubated with either CD55-NEV or the control VV at 1×10³ pfu/sample for 1 hr at 37° C. in the presence of 1:10 dilution of human complement. The mixture was then added to the CV-1 cells and allowed to adhere for 2 hrs at 37° C./5% CO₂ in 300 μL of serum free media. After 2 hrs, the inoculum was removed and 1mL of complete DMEM medium was added to the cells. The cells were then incubated at 37° C./5% CO₂. After 48 hrs cells were fixed and stained with 1% crystal violet/20% EtOH solution for 20 min at room temperature and plaques were counted. The results suggested that CD55-NEV escaped the neutralization more effectively than NEV and VV in the absence or presence of complement (FIG. 13). Based on these results, it is concluded that the CD55-VV disclosed herein can efficiently escape complement/Nab mediated neutralization in vitro.

### EXAMPLE 11

### Construction of FAP-TEA-NEV

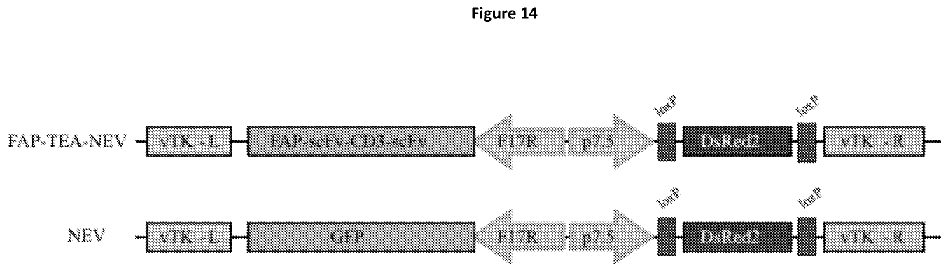

The oncolytic vaccinia virus (VV) construct FAP-TEA-NEV was generated to express a bispecific FAP-CD3 scFv targeting the FAP on cancer associated fibroblast (CAF) and CD3 on T cells. Bispecific FAP-CD3 scFv was optimized and synthesized and cloned into a pMS shuttle plasmid (FIG. 14). The mhFAP -cross reactive single chain variable fragment (scFv M036) was previously generated by phage display from an immunized FAP/knock-out mouse. Human CD3 scFv was derived from OKT3 clone. Vaccinia viruses (Western Reserve strain) expressing secretory bispecific FAP-CD3 scFv (FAP-TEA-NEV) were generated by recombination of a version of pMS shuttle plasmid into the TK gene of the WR VV or NEV. The inserted bispecific FAP-CD3 scFv was expressed under the transcriptional control of the F 17R late promoter to allow for sufficient viral replication before T-cell activation. To construct the recombinant virus BCMA-TEA-NEV, the shuttle vectors pMS were transfected into CV-1 or 293 cells. Cells were then infected with WR VV or NEV at a multiplicity of infection (MOI) of 0.1. After three rounds of plaque selection and amplification to confirm the expression of FAP-CD3, one of the corresponding clones was selected for amplification and purification.

In one embodiment, an amino acid sequence comprising the FAP-CD3 polypeptide is shown in SEQ ID NO:8. An example of an optimized nucleotide sequence for the FAP-CD3 polypeptide, containing signal peptide, FAP scFv, CD3 scFv and linker sequence is shown in SEQ ID NO:17.

### EXAMPLE 12

### Evaluation of FAP-TEA-NEV In Vitro

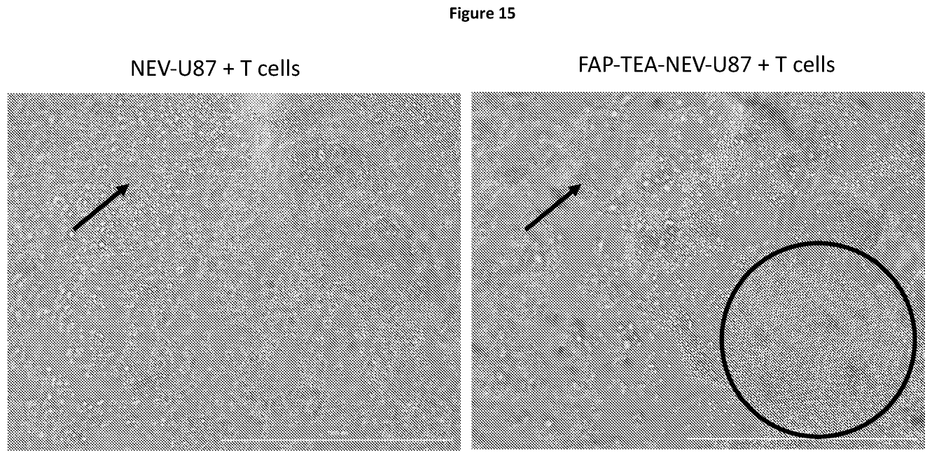

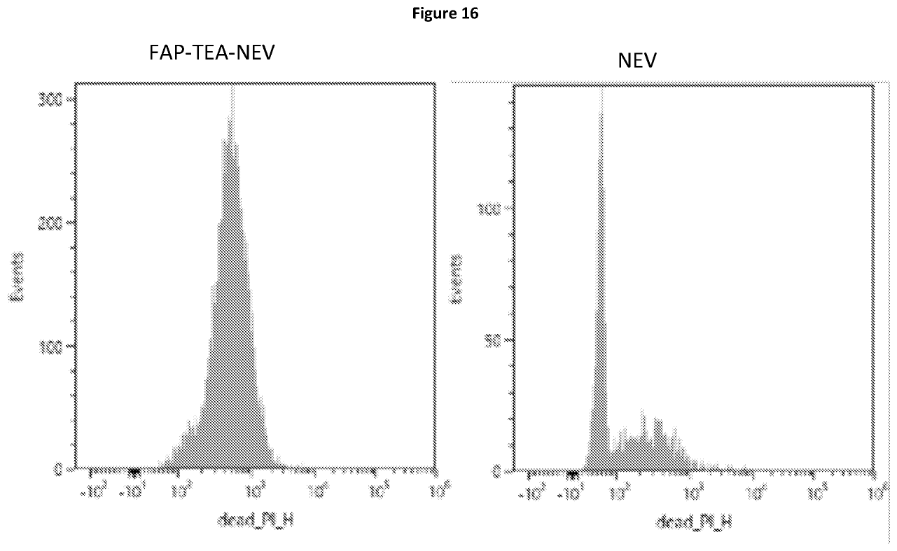

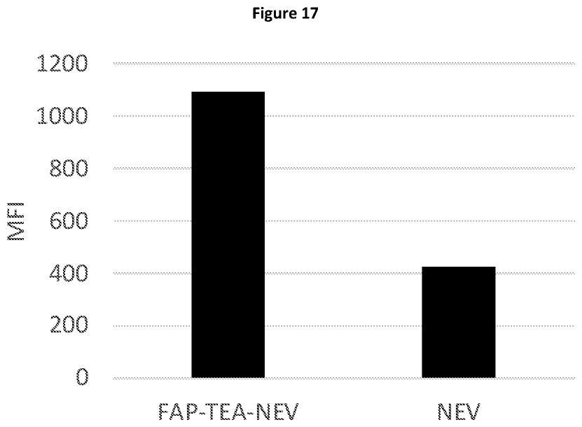

Tumor lysis capacity of FAP-TEA-NEV was investigated. FAP-positive U87 tumor cells were seeded into 96-well plates at 5x10e4 cell number per well. The U87 tumor cells were then infected with FAP-TEA-NEV or NEV at MOI 1, and co-cultured with human T cells at ration of U87:T=1:5. After 48 hrs, cells were observed under microscope. The microscope picture showed that FAP-TEA-VV induced U87 tumor cell lysis and human T cell proliferation effectively compared to NEV (FIG. 15). Cells were stained with apoptosis marker PI and Flow analysis results suggested that FAP-TEA-VV induced U87 tumor apoptosis more effectively than NEV (FIG. 16). FIG. 17 showed the MFI of PI staining of gated U87 tumor cells.

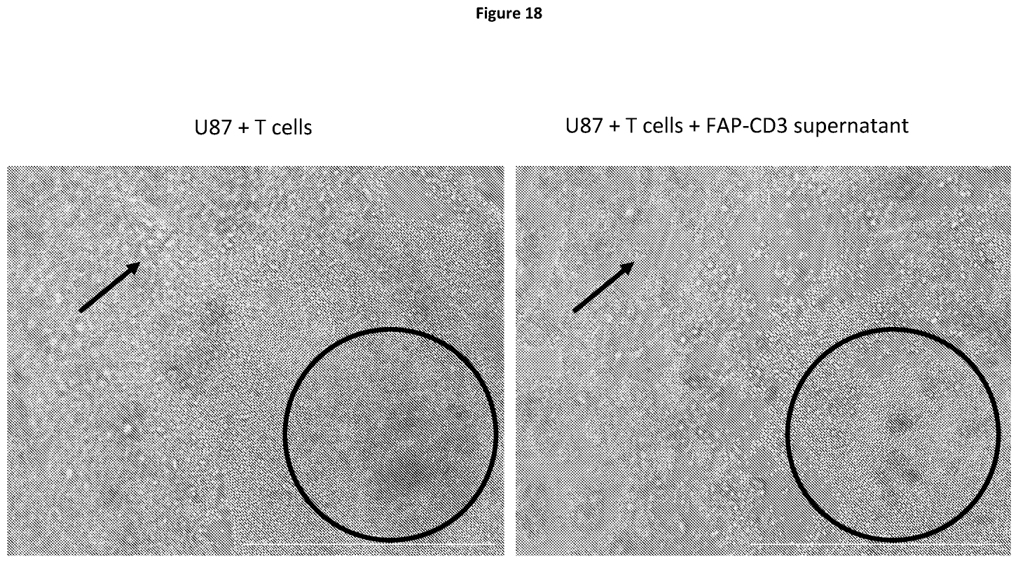

The ability of FAP-TEA-NEV to induce bystander tumor lysis was also investigated. CV-1 cells were infected with FAP-TEA-VV at MOI 1, and the cell culture medium were collected at 24 hours and added to co-culture of FAP-positive U87 tumor cells and human T cells at ratio of U87:T=1:5. U87 tumor cells were seeded into 96-well plates at 5×10e4 cell number per well. After 48 hrs, cells were observed under microscope. The microscope picture showed that FAP-TEA-VV induced U87 tumor cell lysis and human T cell proliferation effectively compared to NEV (FIG. 18).

### EXAMPLE 13

### Construction of BCMA-TEA-NEV

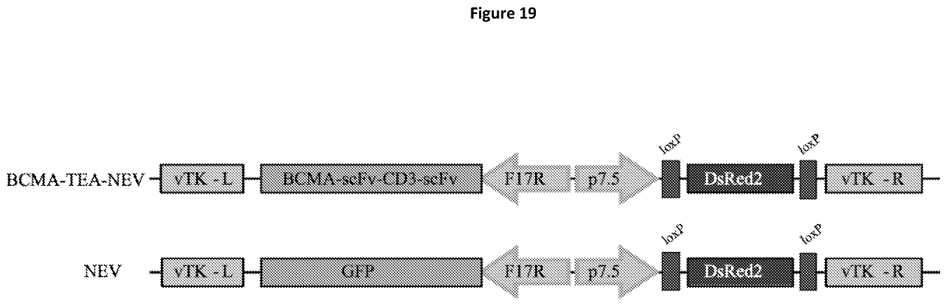

The oncolytic vaccinia virus (VV) construct BCMA-TEA-NEV was generated to express a bispecific BCMA-CD3 scFv targeting the BCMA on multiple myeloma and CD3 on T cells. Bispecific BCMA-CD3 scFv was optimized and synthesized and cloned into a pMS shuttle plasmid (FIG. 19). BCMA scFV was derived from C11D5.3 clone (U.S. Pat. No. 9,034,324B2). Human CD3 scFv was derived from OKT3 clone. Vaccinia viruses (Western Reserve strain) expressing secretory bispecific BCMA-CD3 scFv (BCMA-TEA-NEV) were generated by recombination of a version of pMS shuttle plasmid into the TK gene of the WR vaccinia virus (WR VV) or NEV. The inserted bispecific BCMA-CD3 scFv was expressed under the transcriptional control of the F 17R late promoter to allow for sufficient viral replication before T-cell activation. To construct the recombinant virus BCMA-TEA-NEV, the shuttle vectors pMS were transfected into CV-1 or 293 cells. Cells were then infected with WR VV or NEV at a multiplicity of infection (MOI) of 0.1. After three rounds of plaque selection and amplification to confirm the expression of BCMA-CD3, one of the corresponding clones was selected for amplification and purification.

In one embodiment, an amino acid sequence comprising the BCMA-CD3 scFv is shown in SEQ ID NO:9. An example of an optimized nucleotide sequence for the BCMA-CD3 scFv, containing signal peptide, BCMA scFv, CD3 scFv and linker sequence is shown in SEQ ID NO:18.

### EXAMPLE 14

### Evaluation of BCMA-TEA-NEV In Vitro

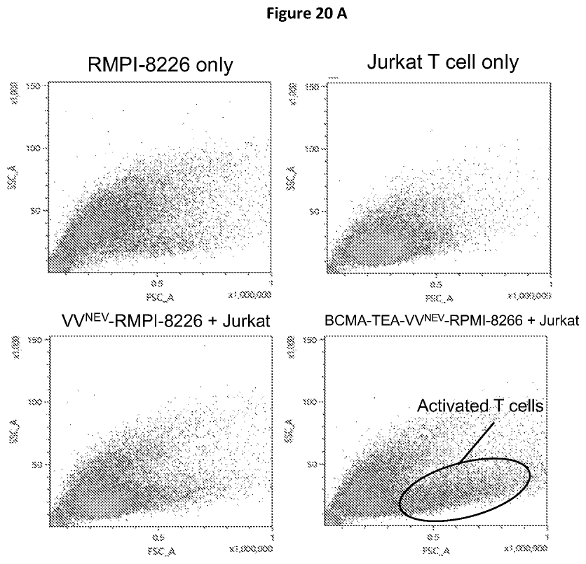

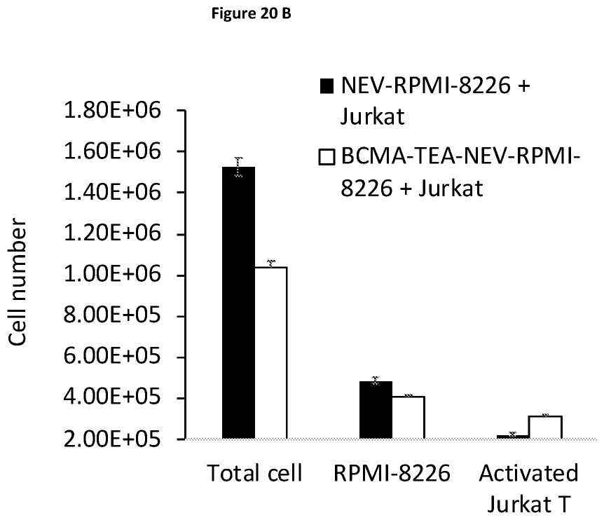

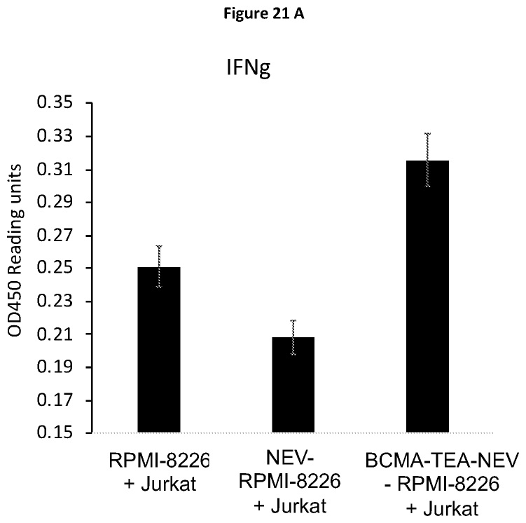

BCMA positive RPMI-8226 MM cell line was infected with BCMA-TEA-NEV or control NEV at MOI 2. After 24 hours, the virus infected RPMI-8226 cells were co-cultured with Jurkat T cells (Invivogen) at ratio of Jurkat T: RPMI-8226=2:1. After 24 hours of incubation, the cells were collected for counting the cell number and flow analysis of cell population. Flow analysis of the cell population suggested Jurkat T cells were significantly activated by BCMA-CD3 (FIG. 20A). FIG. 20B shows the cell number of the RPMI-8266 MM cells and activated Jurkat T cells. The results suggested that BCMA-TEA-NEV significantly induced Jurkat T cell activation and RPMI-8266 MM cell lysis compared to NEV control.

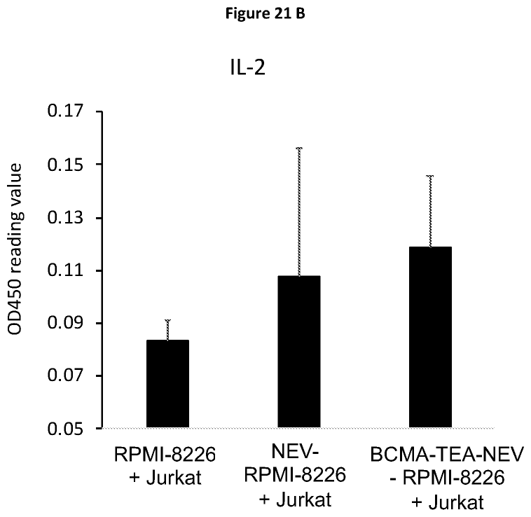

In the above experiment, after 24 hours of incubation, the cells were collected for measurement of cytokines IFNγ (FIG. 21A) and IL2 (FIG. 21B) secretion by ELISA. The results suggested that BCMA-TEA-NEV significantly induced IFNγ and IL2 expression by Jurkat T cells compared to NEV control.

### EXAMPLE 15

### Construction of PD-1-ED-hIgG1-Fc-NEV

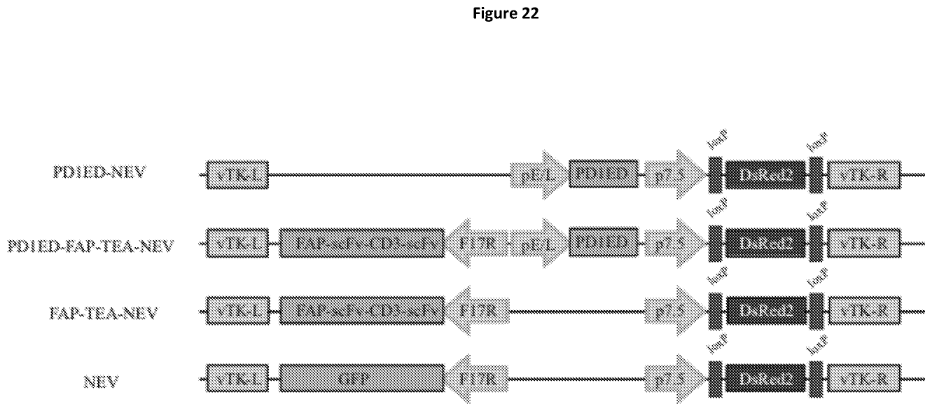

The oncolytic vaccinia virus (VV) construct PD-1-ED-hIgG1-Fc-NEV was generated to express a recombinant protein with the extracellular domain of PD-1 fused to the constant (Fc) domain of immunoglobin-G1 (IgG1). FAP-CD3 is a bispecific molecule targeting the fibroblast activation protein on cancer associated fibroblast and CD3 on T cells. PD-1-ED-hIgG1-Fc was optimized and synthesized and cloned into a pMS shuttle plasmid (FIG. 22). Vaccinia viruses (Western Reserve strain) expressing secretory PD-1-ED-hIgG1-Fc (PD-1-ED-hIgG1-Fc-NEV) or co-expressing secretory PD-1-ED-hIgG1-Fc and FAP-CD3 (PD-1-ED-hIgG1-Fc-FAP-TEA-NEV) were generated by recombination of a version of pMS shuttle plasmid into the TK gene of the WR vaccinia virus (WR VV) or NEV. The inserted PD-1-ED-hIgG1-Fc was expressed under the transcriptional control of the pSE/L promoter. The inserted FAP-CD3 was expressed under the transcriptional control of the F17R late promoter to allow for sufficient viral replication before T-cell activation. To construct the recombinant virus PD-1-ED-hIgG1-Fc-NEV or PD-1-ED-hIgG1-Fc-FAP-TEA-NEV, the shuttle vectors pMS were transfected into CV-1 or 293 cells. Cells were then infected with WR VV or NEV at a multiplicity of infection (MOI) of 0.1. After three rounds of plaque selection and amplification to confirm the expression of PD-1-ED-hIgG1-Fc or FAP-CD3, one of the corresponding clones was selected for amplification and purification.

In one embodiment, an amino acid sequence comprising the PD-1-ED-hIgG1-Fc is shown in SEQ ID NO:10. An example of an optimized nucleotide sequence for the PD-1-ED-hIgG1-Fc, containing signal peptide, PD-1 extracellular domain, human IgG1 hinge and Fc domain is shown in SEQ ID NO:19.

### EXAMPLE 16

### Evaluation of PD1ED-NEV In Vitro

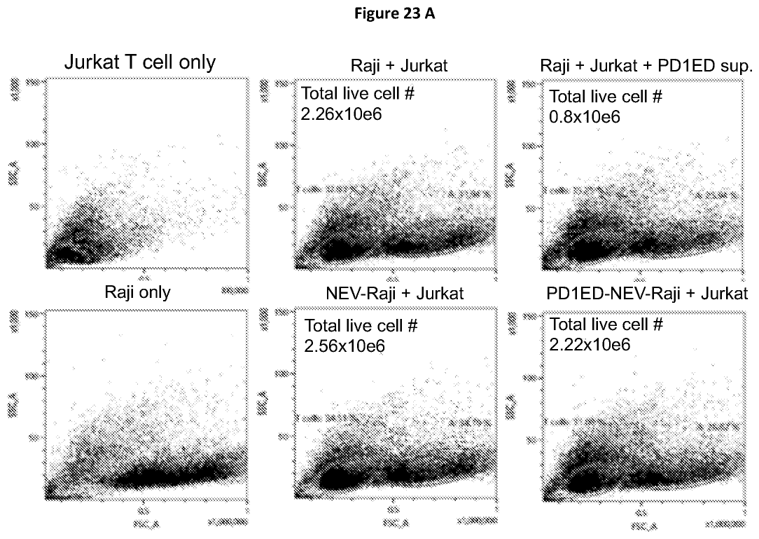

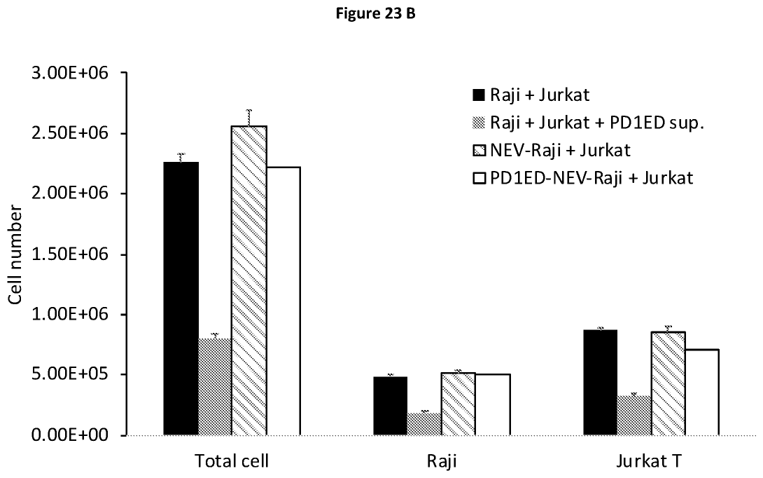

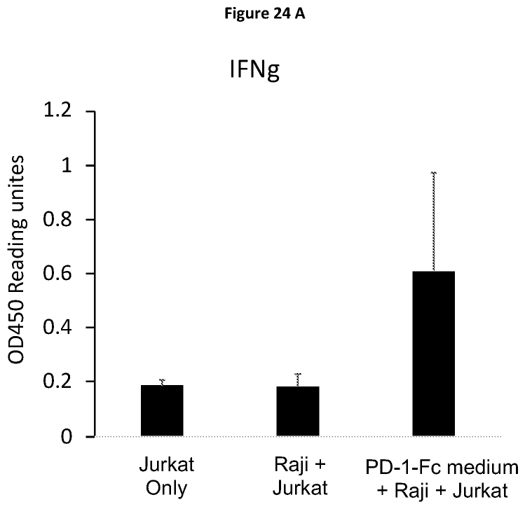

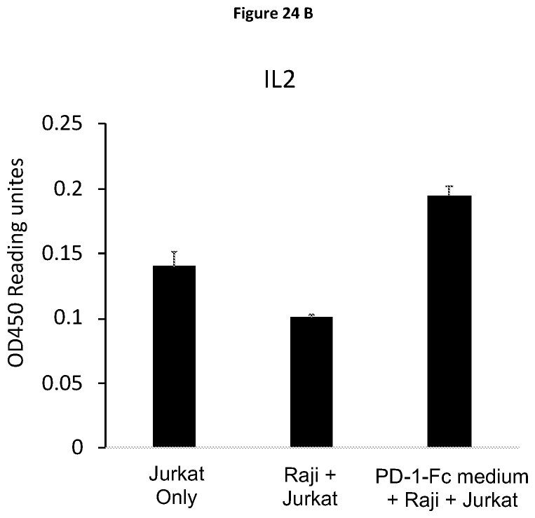

Stable PD-L1-Raji (Invivogen) cell line was infected with PD1ED-NEV or control NEV at MOI 2. After 24 hours, the virus infected PD-L1-Raji cells were co-cultured with NFAT-CD16-Luc reporter Jurkat T cells (Invivogen) at ratio of Jurkat T : PD-L1-Raji=2:1. To investigate the role of the secreted PD-1-ED-Fc, CV-1 cells were infected with BCMA-TEA-NEV at MOI2 and the cell culture medium was collected after 24 hours and added to the co-culture of Raji and Jurkat T cells. After 24 hours of incubation, the cells were collected for flow analysis (FIG. 23A) and counting (FIG. 23B). The results suggested secreted PD-1-ED-Fc effectively induced Raji cell lysis compared to control group. PD-1-ED-Fc also induced significant Jurkat T cell exhaustion (FIG. 19B). NEV infection of Raji has no effects likely because Raji is not susceptiable to VV infection. In the above experiment, after 24 hours of incubation, the cells were collected for measurement of cytokines IFNγ (FIG. 24A) and IL2 (FIG. 24B) secretion by ELISA. The results suggested that secreted PD1ED significantly induced IFNγ and IL2 expression by Jurkat T cells compared to NEV control.

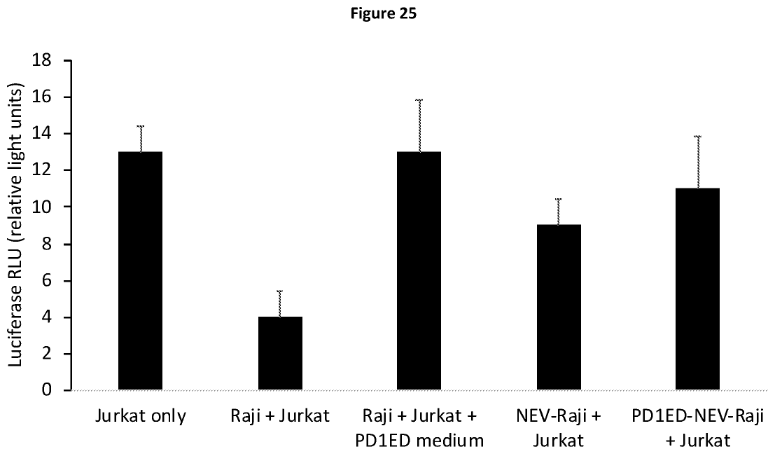

In another experiment, stable PD-L1-Raji (Invivogen) cell line was infected with PD1ED-NEV or control NEV at MOI 2. After 24 hours, the virus infected PD-L1-Raji cells were co-cultured with NFAT-CD16-Luc reporter Jurkat T cells (Invivogen) at ratio of Jurkat T : PD-L1-Raji=2:1. To investigate the role of the secreted PD-1-ED-Fc, CV-1 cells were infected with BCMA-TEA-NEV at MOI2 and the cell culture medium was collected after 24 hours and added to the co-culture of Raji and Jurkat T cells. After 6 hours of incubation, the supernatant was collected for luciferase measurement (FIG. 25). The results suggested secreted PD-1-ED-Fc effectively activated Jurkat T cells compared to control NEV or medium.

What is claimed is:

1. An isolated infectious recombinant vaccinia virus (VV) virion, comprising a heterologous nucleic acid and one or more of:
a) a variant vaccinia virus (VV) H3L protein having at least about 60% amino acid sequence identity to SEQ ID NO:1;
b) a variant vaccinia virus (VV) D8L protein having at least about 60% amino acid sequence identity to SEQ ID NO:2;
c) a variant vaccinia virus (VV) A27L protein having at least about 60% amino acid sequence identity to SEQ ID NO:3;
d) a variant vaccinia virus (VV) L1R protein having at least about 60% amino acid sequence identity to SEQ ID NO:4;
e) a variant vaccinia virus (VV) H3L protein having at least about 60% amino acid sequence identity to SEQ ID NO:5;
f) a variant vaccinia virus (VV) D8L protein having at least about 60% amino acid sequence identity to SEQ ID NO:6 or SEQ ID NO:174;
g) a variant vaccinia virus (VV) H3L protein having at least about 60% amino acid sequence identity to SEQ ID NO:170; and
h) a variant vaccinia virus (VV) D8L protein having at least about 60% amino acid sequence identity to SEQ ID NO:172.

2. The recombinant vaccinia virus (VV) virion of claim 1, wherein said variant VV H3L protein comprises amino acid substitution or deletion at one or more amino acid residues selected from the group consisting of 14, 15, 16, 33, 34, 35, 38, 40, 44, 45, 52, 131, 134, 135, 136, 137, 154, 155, 156, 161, 166, 167, 168, 198, 227, 250, 253, 254, 255, and 256 of SEQ ID NO:1.

3. The recombinant vaccinia virus (VV) virion of claim 1, wherein said variant VV D8L protein comprises amino acid substitution or deletion at one or more amino acid residues selected from the group consisting of 44, 48, 98, 108, 117, and 220 of SEQ ID NO:2.

4. The recombinant vaccinia virus (VV) virion of claim 1, wherein said variant VV A27L protein comprises amino acid substitution or deletion at one or more amino acid residues selected from the group consisting of 27, 30, 32, 33, 34, 35, 36, 37, 39, 40, 107, 108, and 109 of SEQ ID NO:3.

5. The recombinant vaccinia virus (VV) virion of claim 1, wherein said variant VV L1R protein comprises amino acid substitution or deletion at one or more amino acid residues selected from the group consisting of 25, 27, 31, 32, 33, 35, 58, 60, 62, 125, and 127 of SEQ ID NO:4.

6. The recombinant vaccinia virus (VV) virion of claim 1, wherein said variant VV H3L protein comprises amino acid substitution or deletion at one or more amino acid residues selected from the group consisting of 14, 15, 16, 33, 34, 35, 38, 40, 44, 45, 52, 131, 132, 134, 135, 136, 137, 154, 155, 156, 161, 166, 167, 168, 195, 198, 199, 227, 250, 251, 252, 253, 254, 255, 256, 258, 262, 264, 266, 268, 272, 273, 275, and 277 of SEQ ID NO:170.

7. The recombinant vaccinia virus (VV) virion of claim 1, wherein said variant VV D8L protein comprises amino acid substitution or deletion at one or more amino acid residues selected from the group consisting of 43, 44, 48, 53, 54, 55, 98, 108, 109, 144, 168, 177, 196, 199, 203, 207, 212, 218, 220, 222, and 227 of SEQ ID NO:172.

8. The recombinant vaccinia virus (VV) virion of claim 1, wherein said heterologous nucleic acid encodes a domain of a regulator of complement activation.

9. The recombinant vaccinia virus (VV) virion of claim 8, wherein said regulator of complement activation is selected from the group consisting of CD55, CD59, CD46, CD35, factor H, and C4-binding protein.

10. The recombinant vaccinia virus (VV) virion of claim 1, wherein said heterologous nucleic acid encodes a CD55 polypeptide comprising the amino acid sequence of SEQ ID NO:7.

11. The recombinant vaccinia virus (VV) virion of claim 1, wherein said heterologous nucleic acid encodes a bi-specific polypeptide that binds to a first antigen on immune cells and a second antigen on tumor cells.

12. The recombinant vaccinia virus (VV) virion of claim 11, wherein said first antigen on immune cells is selected from the group consisting of CD3, CD4, CD5, CD8, CD16, CD28, CD40, CD64, CD89, CD134, CD137, NKp46, and NKG2D.

13. The recombinant vaccinia virus (VV) virion of claim 11, wherein said second antigen on tumor cells is selected from the group consisting of fibroblast activation protein (FAP), and tumor antigens on multiple myeloma.

14. The recombinant vaccinia virus (VV) virion of claim 11, wherein the bi-specific polypeptide is a bi-specific scFvs, said first antigen is human CD3e, said second antigen is human FAP, and said bi-specific polypeptide having the amino acid sequence of SEQ ID NO:8.

15. The recombinant vaccinia virus (VV) virion of claim 13, wherein the tumor antigens on multiple myeloma are selected from the group consisting of B-cell maturation antigen (BCMA), CD19, CD38, SLAMF7, CD26, LIGHT/TNFSF14, integrin beta7, CD138, KIRs, EGFR, PD-1/PD-L1, TGIT, CD56, CS1, NKG2D, TACI, and CD44v6.

16. The recombinant vaccinia virus (VV) virion of claim 11, wherein the bi-specific polypeptide is a bi-specific scFvs, said first antigen is human CD3e, said second antigen is human BCMA, and said bi-specific polypeptide having the amino acid sequence of SEQ ID NO:9.

17. The recombinant vaccinia virus (VV) virion of claim 1, wherein said heterologous nucleic acid encodes a fusion polypeptide comprising an immune checkpoint molecule.

18. The recombinant vaccinia virus (VV) virion of claim 17, wherein said immune checkpoint molecule is selected from the group consisting of PD-1, PD-L1, PD-L2, CD47, CXCR4, CSF1R, LAG-3, TIM-3, HHLA2, BTLA, CTLA-4, TIGIT, VISTA, B7-H4, CD160, 2B4, and CD73.

19. The recombinant vaccinia virus (VV) virion of claim 1, wherein said heterologous nucleic acid encodes a fusion polypeptide comprising human PD-1 extracellular domain and a human IgG1 Fc domain, said fusion polypeptide having the amino acid sequence of SEQ ID NO:10.

20. The recombinant vaccinia virus (VV) virion of claim 1, wherein the VV exhibits resistance to neutralizing antibodies compared to that exhibited by wild type VV.

21. The recombinant vaccinia virus (VV) virion of claim 1, wherein the VV exhibits increased transduction of mammalian cells in the presence of VV neutralizing antibodies compared to transduction of mammalian cells by wild type VV.

22. A method of delivering a gene product to a subject in need thereof, comprising administering to the subject an effective amount of the recombinant vaccinia virus (VV) virion of claim 1, said gene product is encoded by said heterologous nucleic acid.

23. A pharmaceutical composition comprising the recombinant vaccinia virus (VV) virion of claim 1 and a pharmaceutically acceptable carrier.

24. A method of treating cancer in a subject, comprising administering to the subject an effective amount of the pharmaceutical composition of claim 23.

25. The method of claim 24, wherein the pharmaceutical composition is administered to the subject systemically, intravenously, or through injection, inhalant, infusion, implantation, parenteral administration, or enteral administration.

26. The method of claim 24, wherein the subject is a human or an animal.

27. A library comprising one or more variant vaccinia virus (VV) virions, each of the one or more variant VV virions comprises one or more variant VV proteins, wherein at least one of said variant VV proteins comprises an amino acid sequence having at least one amino acid substitution or deletion relative to the amino acid sequence of a corresponding wild type VV protein.

28. The library of claim 27, wherein at least one of the one or more variant VV proteins is selected from the group consisting of H3L protein, D8L protein, A27L protein, and L1R protein.

29. The library of claim 27, wherein at least one of the one or more variant VV proteins comprises an amino acid sequence having at least one amino acid substitution or deletion relative to the amino acid sequence of one of SEQ ID No:5, SEQ ID No:6, or SEQ ID No:174.

30. A recombinant vaccinia virus (VV) virion derived from the library of claim 27, comprising a heterologous nucleic acid and one or more variant VV proteins, wherein at least one of said variant VV proteins comprises an amino acid sequence having at least one amino acid substitution or deletion relative to the amino acid sequence of a corresponding wild type VV protein.

31. The recombinant vaccinia virus (VV) virion of claim 30, wherein said heterologous nucleic acid encodes a domain of a regulator of complement activation.

32. The recombinant vaccinia virus (VV) virion of claim 31, wherein said regulator of complement activation is selected from the group consisting of CD55, CD59, CD46, CD35, factor H, and C4-binding protein.

33. The recombinant vaccinia virus (VV) virion of claim 31, wherein said heterologous nucleic acid encodes a CD55 polypeptide comprising the amino acid sequence of SEQ ID NO:7.

34. The recombinant vaccinia virus (VV) virion of claim 30, wherein said heterologous nucleic acid encodes a bi-specific polypeptide that binds to a first antigen on immune cells and a second antigen on tumor cells.

35. The recombinant vaccinia virus (VV) virion of claim 34, wherein said first antigen on immune cells is selected from the group consisting of CD3, CD4, CD5, CD8, CD16, CD28, CD40, CD64, CD89, CD134, CD137, NKp46, and NKG2D.

36. The recombinant vaccinia virus (VV) virion of claim 34, wherein said second antigen on tumor cells is selected from the group consisting of fibroblast activation protein (FAP), and tumor antigens on multiple myeloma.

37. The recombinant vaccinia virus (VV) virion of claim 34, wherein the bi-specific polypeptide is a bi-specific scFvs, said first antigen is human CD3e, said second antigen is human FAP, and said bi-specific polypeptide having the amino acid sequence of SEQ ID NO:8.

38. The recombinant vaccinia virus (VV) virion of claim 36, wherein the tumor antigens on multiple myeloma are selected from the group consisting of B-cell maturation antigen (BCMA), CD19, CD38, SLAMF7, CD26, LIGHT/TNFSF14, integrin beta7, CD138, KIRs, EGFR, PD-1/PD-L1, TGIT, CD56, CS1, NKG2D, TACI, and CD44v6.

39. The recombinant vaccinia virus (VV) virion of claim 34, wherein the bi-specific polypeptide is a bi-specific scFvs, said first antigen is human CD3e, said second antigen is human BCMA, and said bi-specific polypeptide having the amino acid sequence of SEQ ID NO:9.

40. The recombinant vaccinia virus (VV) virion of claim 30, wherein said heterologous nucleic acid encodes a fusion polypeptide comprising an immune checkpoint molecule.

41. The recombinant vaccinia virus (VV) virion of claim 40, wherein said immune checkpoint molecule is selected from the group consisting of PD-1, PD-L1, PD-L2, CD47, CXCR4, CSF1R, LAG-3, TIM-3, HHLA2, BTLA, CTLA-4, TIGIT, VISTA, B7-H4, CD160, 2B4, and CD73.

42. The recombinant vaccinia virus (VV) virion of claim 40, wherein said heterologous nucleic acid encodes a fusion polypeptide comprising human PD-1 extracellular domain and a human IgG1 Fc domain, said fusion polypeptide having the amino acid sequence of SEQ ID NO:10.

43. The recombinant vaccinia virus (VV) virion of claim 30, wherein the VV virion exhibits resistance to neutralizing antibodies compared to wild type VV.

44. The recombinant vaccinia virus (VV) virion of claim 30, wherein the VV virion exhibits increased transduction of mammalian cells in the presence of VV neutralizing antibodies compared to transduction of mammalian cells by wild type VV.

45. A method of delivering a gene product to a subject in need thereof, comprising administering to the individual an effective amount of the recombinant vaccinia virus (VV) virion of claim 30, wherein the gene product is encoded by the heterologous nucleic acid carried by said variant VV virion.

46. A pharmaceutical composition comprising the recombinant vaccinia virus (VV) virion of claim 30 and a pharmaceutically acceptable carrier.

47. A method of treating cancer in a subject, comprising administering to the subject an effective amount of the pharmaceutical composition of claim 46.

48. The method of claim 47, wherein the pharmaceutical composition is administered to the subject systemically, intravenously, or through injection, inhalant, infusion, implantation, parenteral administration, or enteral administration.

49. The method of claim 47, wherein the subject is a human or an animal.

50. A recombinant vaccinia virus H3L protein having at least about 60% amino acid sequence identity to one of SEQ ID NOs:1, 5 or 170.

51. A recombinant vaccinia virus D8L protein having at least about 60% amino acid sequence identity to one of SEQ ID NOs:6, 172 or 174.


SEQUENCE LISTING (174 sequences)

SEQ ID NO: 1 - protein, 324 aa, Artificial Sequence
  SEQ ID NO1, Mutant H3L amino acid sequence
MAAAKTPVIVVPVAAALPSETFPNVHEHINDQAAADVADAEVMAAKRNVVVAKDDPDHYKDYAFIQWTGGNIRNDDKYTHFFSGFCNTMCTEETKRNIARHLALWDSNFFTELENKKVEYVVIVENDNVIAAIAFLAPVLKAMHDKKIDILQMAEAITGNAVKTEAAADKNHAIFTYTGGYDVSLSAYIIRVTTALNIADEIIKSGGLSSGFYFEIARIENEMKINAQILDNAAKYVEHDPRLVAEHRFANMAAAAWSRIGTAATKRYPGVMYAFTTPLISFFGLFDINVIGLIVILFIMFMLIFNVKSKLLWFLTGTFVTAFI

SEQ ID NO: 2 - protein, 304 aa, Artificial Sequence
  SEQ ID NO2, mutant D8L amino acid sequence
MPQQLSPINIETKKAISNARLKPLDIHYNESKPTTIQNTGALVAINFAGGYISGGFLPNEYVLSSLHIYWGKEDDYGSNHLIDVYKYSGEINLVHWNAKKYSSYEEAAKHDDGLIIISIFLQVLDHKNVYFQKIVNQLDSIRSANTSAPFDSVFYLDNLLPSKLDYFTYLGTTINHSADAVWIIFPTPINIHSDQLSKFRTLLSSSNHDGKPHYITENYANPYKLNDDTQVYYSGEIIRAATTSPARENYFMRWLSDLRETCFSYYQKYIEENKTFAIIAIVFVFILTAILFFMSRRYSREKQN

SEQ ID NO: 3 - protein, 110 aa, Artificial Sequence
  SEQ ID NO3, mutant A27L amino acid sequence
MDGTLFPGDDDLAIPATEFFSTKAAKAPEDKAADAAAAAADDNEETLKQRLTNLEKKITNVTTKFEQIEKCCKRNDEVLFRLENHAETLRAAMISLAKKIDVQTGRAAAE

SEQ ID NO: 4 - protein, 250 aa, Artificial Sequence
  SEQ ID NO4, mutant L1R amino acid sequence
MGAAASIQTTVNTLSERISSKLEQAAAASAAAACAIEIGNFYIRQNHGCNLTVKNMCAAAAAAQLDAVLSAATETYSGLTPEQKAYVPAMFTAALNIQTSVNTVVRDFENYVKQTCNSSAVVDNALAIQNVIIDECYGAPGSPTNLEFINTGSSKGNCAIKALMQLTTKATTQIAPKQVAGTGVQFYMIVIGVIILAALFMYYAKRMLFTSTNDKIKLILANKENVHWTTYMDTFFRTSPMVIATTDMQN

SEQ ID NO: 5 - protein, 324 aa, Artificial Sequence
  SEQ ID NO5, mutant H3L amino acid sequence
MAAAKTPVIVVPVIDRLPSETFPNVHEHINDQKFDDVKDNEVMAEKRNVVVVKDDPDHYKDYAFIQWTGGNIRNDDKYTHFFSGFCNTMCTEETKRNIARHLALWDSNFFTELENKKVEYVVIVENDNVIEDITFLRPVLKAMHDKKIDILQMREIITGNKVKTELVMDKNHAIFTYTGGYDVSLSAYIIRVTTALNIVDEIIKSGGLSSGFYFEIARIENEMKINRQILDNAAKYVEHDPRLVAEHRFGWMKPNFWFRIGPATVIRCPGVKNANTAPLISFFGLFDINVIGLIVILFIMFMLIFNVKSKLLWFLTGTFVTAFI

SEQ ID NO: 6 - protein, 304 aa, Artificial Sequence
  mutant D8L amino acid sequence (Alternate for this sequence Seq No. 174 - only different at position 43)
MPQQLSPINIETKKAISNARLKPLDIHYNESKPTTIQNTGKLFWINFKGGYISGWFLPNEYVLSSLHIYWGKEDDYGSNHLIDVYKYSGEINLVHWNKKKYSSYEEAKKHDDGLIIISIFLQVLDHKNVYFQKIVNQLDSIRSTNTSAPFDSVFYLDNLLPSKLDYFSYLGTTINHYADAVWIIFPTPINIHSDQLSKYRTLSSSSNHDGKTHYITECYRNLYKLNGDTQVYYSGEIIRAATTSPARENYFMRWLSDLRETCFSYYQKYIEENKTFAIIAIVFVFILTAILFFMSRRYSREKQN

SEQ ID NO: 7 - protein, 375 aa, Artificial Sequence
  SEQ ID NO7, CD55-A27 amino acid sequence
MDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGGGGSGGGGSGGGGSDGTLFPGDDDLAIPATEFFSTKAAKAPEDKAADAAAAAADDNEETLKQRLTNLEKKITNVTTKFEQIEKCCKRNDEVLFRLENHAETLRAAMISLAKKIDVQTGRAAAE

SEQ ID NO: 8 - protein, 526 aa, Artificial Sequence
  SEQ ID NO8, FAP-CD3 amino acid sequence
MDWIWRILFLVGAATGAHSQVQLKQSGAELVKPGASVKLSCKTSGYTFTENIIHWVKQRSGQGLEWIGWFHPGSGSIKYNEKFKDKATLTADKSSSTVYMELSRLTSEDSAVYFCARHGGTGRGAMDYWGQGTSVTVSSGGGGSGGGGSGGSAQILMTQSPASSVVSLGQRATISCRASKSVSTSAYSYMHWYQQKPGQPPKLLIYLASNLESGVPPRFSGSGSGTDFTLNIHPVEEEDAATYYCQHSRELPYTFGGGTKLEIKRAGSGGGGSGGGGSGGGGSVDDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 9 - protein, 515 aa, Artificial Sequence
  SEQ ID NO9, BCMA-CD3 scFv amino acid sequence
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSVDDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 10 - protein, 402 aa, Artificial Sequence
  SEQ ID NO10, PD-1-ED-hIgG1-Fc amino acid sequence
MQIPQAPWPVVWAVLQLGWRPGWFLDSPDRPWNPPTFFPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 324 aa, Artificial Sequence
  SEQ ID NO11, Mutant H3L amino acid sequence
MAAAKTPVIVVPVAAALPSETFPNVHEHINDQKFDDVADNEVMAAKRNVVVAKDDPDHYKDYAFIQWTGGNIRNDDKYTHFFSGFCNTMCTEETKRNIARHLALWDSNFFTELENKKVEYVVIVENDNVIAAIAFLAPVLKAMHDKKIDILQMAEAITGNKVKTELVADKNHAIFTYTGGYDVSLSAYIIRVTTALNIADEIIKSGGLSSGFYFEIARIENEMKINRQILDNAAKYVEHDPRLVAEHRFANMAAAAWSRIGTAATKRYPGVMYAFTTPLISFFGLFDINVIGLIVILFIMFMLIFNVKSKLLWFLTGTFVTAFI

SEQ ID NO: 12 - DNA, 2548 bp, Artificial Sequence
  SEQ ID NO12,  Nucleotide sequences for mutated H3L gene
gaagaactcatagatcacgaacatgtgcaatacaaaataaattgttacaatattctaagatatcatttattgccagacagtgacgtgtttgtatattttagtaattcattaaacagagaagcattggaatacgcattttatatctttttgtcgaaatatgtaaatgtgaaacaatggatagacgaaaatataactcgtattaaagagttgtatatgattaatttcaataactaaatggcggcggcgaaaactcctgttattttaatttattatgatatttaaatatcgcctaatatggcggcggcgaaaactcctgttattgttgtgccagttgctgctgcacttccatcagaaacatttcctaatgttcatgagcatattaatgatcagaagttcgatgatgtagcggacaacgaagttatggcagcaaaaagaaatgttgtggtagccaaggatgatccagatcattacaaggattatgcgtttatacagtggactggaggaaacattagaaatgatgacaagtatactcacttcttttcagggttttgtaacactatgtgtacagaggaaacgaaaagaaatatcgctagacatttagccctatgggattctaatttttttaccgagttagaaaataaaaaggtagaatatgtagttattgtagaaaacgataacgttattgcggctattgcgtttcttgctcccgtcttgaaggcaatgcatgacaaaaaaatagatatcctacagatggcagaagctattacaggcaataaagttaaaaccgagcttgtagcggacaaaaatcatgccatattcacatatacaggagggtatgatgttagcttatcagcctatattattagagttactacggcgctgaacatcgcagatgaaattataaagtctggaggtctatcatcgggattttattttgaaatagccagaattgaaaacgaaatgaagatcaataggcagatactggataatgccgccaaatatgtagaacacgatccccgacttgttgcagaacaccgtttcgcaaacatggcagcggctgcttggtctagaataggaacggcagctactaaacgttatccaggagttatgtacgcgtttactactccactgatttcattttttggattgtttgatattaatgttataggtttgattgtaattttgtttattatgtttatgctcatctttaacgttaaatctaaactgttatggttccttacaggaacattcgttaccgcatttatctaataatccaaacccacccgctttttatagtaagtttttcacccataaataataaatacaataattaatttctcgtaaaagtagaaaatatattctaatttattgcacggtaaggaagtagatcataactcgagataacttcgtataatgtatgctatacgaagttattactagcgctaccggtcgccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacttactagcgctcaataacttcgtataatgtatgctatacgaagttattaatacaggaacattcgttaccgcatttatctaacactattccatattactaaaatcggaacaccaatgcggtgacataaaataaccgctataacctaattcatttaacatctcattaccacaagtaataacattattagacttgtgttttatcaaatactgacaaaattgttgagcagatggatcgacctttgccgcctttttaaccatccacgcgtctccagtacctcgcctaatagcttgcggcagatatgttttcttatccaatcgcatagctataaaataggcgccgaaatccacacatttgaattcgaatatatcatcc

SEQ ID NO: 13 - DNA, 2467 bp, Artificial Sequence
  SEQ ID NO13,  Nucleotide sequences for mutated D8L gene
agaatctgaattttgttgagataatatcgcctggaacgcgaatgaagttcttctagctcctattaacggatatccgtcacttgttatacacgcagcaaacacgtgcgtgtcttttgatcttggaatatcttttattcgtttaatagatattaattctctaggagtttcaaatatcacttcctcatccattgtaattcccatactaagagctatttttaaacagttatcatttcatttttactatgccgcaacaactatctcctattaaatagaaactattaatttattatgatatttaaatatcgcctaatatgccgcaacaactatctcctattaatatagaaactaaaaaagcaatttctaacgcgcgattgaagccgttagacatacattataatgagtcgaaaccaaccactatccagaacactggagcactagtagcgattaattttgcaggaggatatataagtggagggtttctccccaatgaatatgtgttatcatcactacatatatattggggaaaggaagacgattatggatccaatcacttgatagatgtgtacaaatactctggagagattaatcttgttcattggaatgcgaaaaaatatagttcttatgaagaggcagcaaaacacgatgatggacttatcattatttctatattcttacaagtattggatcataaaaatgtatattttcaaaagatagttaatcaattggattccattagatccgccaatacgtctgcaccgtttgattcagtattttatctagacaatttgctgcctagtaagttggattattttacatatctaggaacaactatcaaccactctgcagacgctgtatggataatttttccaacgccaataaacattcattctgatcaactatctaaattcagaacactattgtcgtcgtctaatcatgatggaaaaccgcattatataacagagaactatgcaaatccgtataaattgaacgacgacacgcaagtatattattctggggagattatacgagcagcaactacctctccagcgcgcgagaactattttatgagatggttgtccgatttgagagagacatgtttttcatattatcaaaaatatatcgaagagaataaaacattcgcaattattgccatagtattcgtgtttatacttaccgctattctcttttttatgagtcgacgatattcgcgagaaaaacaaaactagtaatccaaacccacccgctttttatagtaagtttttcacccataaataataaatacaataattaatttctcgtaaaagtagaaaatatattctaatttattgcacggtaaggaagtagatcataactcgagataacttcgtataatgtatgctatacgaagttattactagcgctaccggtcgccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacttactagcgctcaataacttcgtataatgtatgctatacgaagttattaaatagtattcgtgtttatacttaccgctattctcttttttatgagtcgacgatattcgcgagaaaaacaaaactagattcgataccttgttgagcctccattagaacggcagtgacttcgctgccattgtcatacgcattaccatttcgaaaaaagcagtactttgaatcgctaaatgatacagtacccgaatctctacttagtttacagattaaatctccacattgaatagttacatttgattcatcttcgatgtttaatgttcctctgactatatccccaacg

SEQ ID NO: 14 - DNA, 1897 bp, Artificial Sequence
  SEQ ID NO14,  Nucleotide sequences for mutated A27L gene
aaaagtggagatgtgtggtttatccaggaaacggttttgtatccgcttccatatttggatttcaggcagaagttggacccaataatactagatccattagaaaatttaacacgatgcaacaatgtatagactttacattttctgatgttattaacatcgatatttataatccatgtgttgtaccaaatataaataacgcagagtgtcagtttctaaaatctgtactttaaatggacggaactcttttccccggagatgacttaatattttgttaattaaaattatatttataaaatattatataataaatggacggaactcttttccccggagatgacgatcttgcaattccagcaactgaatttttttctacaaaggctgctaaagcgccagaggataaagccgcagacgctgctgcagccgctgcagacgacaatgaggaaactctcaaacaacggctaactaatttggaaaaaaagattactaatgtaacaacaaagtttgaacaaatagaaaagtgttgtaaacgcaacgatgaagttctatttaggttggaaaatcacgctgaaactctaagagcggctatgatatctctggctaaaaagattgatgttcagactggacgggccgcagctgagtaataatccaaacccacccgctttttatagtaagtttttcacccataaataataaatacaataattaatttctcgtaaaagtagaaaatatattctaatttattgcacggtaaggaagtagatcataactcgagataacttcgtataatgtatgctatacgaagttattactagcgctaccggtcgccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagactagcgctcaataacttcgtataatgtatgctatacgaagttatgttcagactggacggcgcccatatgagtaataacttaactcttttgttaattaaaagtatattcaaaaaatgagttatataaatggcgaacattataaatttatggaacggaattgtaccaacggttcaagatgttaatgttgcgagcattactgcgtttaaatctatgatagatgaaacatgggataaaaaaatcgaagcaaatacatgcatcagtagaaaacatagaaacattattcacgaagttattagggactttatgaaagcctatcctaaaatggatgagaataaaaaatctccat

SEQ ID NO: 15 - DNA, 2244 bp, Artificial Sequence
  SEQ ID NO15,  Nucleotide sequences for mutated L1R gene
aatattgtacgatgtaatactagcgtgaacaacttacagatggataaaacttcctcattaagattgtcatgtggattaagcaatagtgatagattttctactgttcccgtcaatagagcaaaagtagttcaacataatattaaacactcgttcgacctaaaattgcatttgatcagtttattatctctcttggtaatatggatactaattgtagctatttaaatgggtgccgcggcaagcttaatattttgttaattaaaattatatttataaaatattatataataaatgggtgccgcggcaagcatacagacgacggtgaatacactcagcgaacgtatctcgtctaaattagaacaagcagcggctgctagtgctgcagcagcatgtgctatagaaatcggaaatttttatatccgacaaaaccatggatgtaacctcactgttaaaaatatgtgcgctgcggccgcggctgctcagttggatgctgtgttatcagccgctacagaaacatatagtggattaacaccggaacaaaaagcatacgtgccagctatgtttactgctgcgttaaacattcagacgagtgtaaacactgttgttagagattttgaaaattatgtgaaacagacttgtaattctagcgcggtcgtcgataacgcattagcgatacaaaacgtaatcatagatgaatgttacggagccccaggatctccaacaaatttggaatttattaatacaggatctagcaaaggaaattgtgccattaaggcgttgatgcaattgacgactaaggccactactcaaatagcacctaaacaagttgctggtacaggagttcagttttatatgattgttatcggtgttataatattggcagcgttgtttatgtactatgccaagcgtatgttgttcacatccaccaatgataaaatcaaacttattttagccaataaggaaaacgtccattggactacttacatggacacattctttagaacttctccgatggttattgctaccacggatatgcaaaactgataatccaaacccacccgctttttatagtaagtttttcacccataaataataaatacaataattaatttctcgtaaaagtagaaaatatattctaatttattgcacggtaaggaagtagatcataactcgagataacttcgtataatgtatgctatacgaagttattagcgctaccggtcgccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattgactagcgctcaataacttcgtataatgtatgctatacgaagttatattgctaccacggatatgcaaaactgaaaatatattgataatattttaatagattaacatggaagttatcactgatcgtctagacgatatagtgaaacaaaatatagcggatgaaaaatttgtagattttgttatacacggtctagagcatcaatgtcctgctatacttcgaccattaattaggttgtttattgatatactattatttgttatagtaatttatatttttacggtacgtctagtaagtagaaa

SEQ ID NO: 16 - DNA, 1131 bp, Artificial Sequence
  SEQ ID NO16,  Nucleotide sequences for CD55- A27
atggattgtggactgccccccgacgtccctaacgctcaacccgctctggaaggcagaacatccttccccgaagacaccgtgatcacctacaagtgtgaggagagcttcgtcaagatccccggcgagaaggatagcgtcatctgtctgaagggaagccaatggtccgacatcgaagagttctgcaacagaagctgtgaggtgcctaccagactgaacagcgcttctctgaagcagccttacatcacccagaactacttccccgtgggcaccgtggtggagtacgagtgcagacccggatacagaagagagccttctctgagccccaagctgacatgcctccagaacctcaagtggagcaccgctgtggagttttgcaagaagaagagctgccccaatcccggcgagattagaaacggccagattgacgtgcccggcggcattctgtttggcgccaccatcagcttcagctgcaacaccggctacaagctgtttggaagcaccagctccttctgtctgatcagcggctccagcgtccagtggagcgatcctctgcccgagtgtagggagatctactgccccgcccctcctcaaatcgacaacggcattatccaaggcgagagggatcactacggctatagacagagcgtcacctacgcttgcaacaagggattcaccatgatcggcgagcactccatctactgcacagtcaacaacgacgagggagaatggagcggccctcctcccgagtgtaggggcggcggcggcagcggcggcggcggcagcggcggcggcggcagcgacggaactcttttccccggagatgacgatcttgcaattccagcaactgaatttttttctacaaaggctgctaaagcgccagaggataaagccgcagacgctgctgcagccgctgcagacgacaatgaggaaactctcaaacaacggctaactaatttggaaaaaaagattactaatgtaacaacaaagtttgaacaaatagaaaagtgttgtaaacgcaacgatgaagttctatttaggttggaaaatcacgctgaaactctaagagcggctatgatatctctggctaaaaagattgatgttcagactggacgggccgcagctgagtaataa

SEQ ID NO: 17 - DNA, 1581 bp, Artificial Sequence
  SEQ ID NO17,  Nucleotide sequences for FAP-CD3
atggactggatctggcgcatcctcttcctcgtcggcgctgctaccggcgctcattctcaggtgcagctgaagcagtctggagctgaactggtgaaacccggggcatcagtgaagctgtcctgcaagacttctggctacaccttcactgaaaatattatacactgggtaaagcagaggtctgggcagggtcttgagtggattgggtggtttcaccctggaagtggtagtataaagtacaatgagaaattcaaggacaaggccacattgactgcggacaaatcctccagcacagtctatatggagcttagtagattgacatctgaagactctgcggtctatttctgtgcaagacacggaggaactgggcgaggagctatggactactggggtcaaggaacctcagtcaccgtctcgagtggtggaggcggttcaggcggaggtggctctggcggtagtgcacaaattctgatgacccagtctcctgcttcctcagttgtatctctggggcagagggccaccatctcatgcagggccagcaaaagtgtcagtacatctgcctatagttatatgcactggtaccaacagaaaccaggacagccacccaaactcctcatctatcttgcatccaacctagaatctggggtccctcccaggttcagtggcagtgggtctgggacagacttcaccctcaacatccaccctgtggaggaggaggatgctgcaacctattactgtcagcacagtagggagcttccgtacacgttcggaggggggaccaagctggaaataaaacgggcgggatccggaggaggaggatctggaggaggaggaagtggcgggggaggctcagtcgacgatatcaagctgcagcagtctggagcagagctggctagaccaggagcatcagtgaaaatgagctgtaagacctccggctatacattcactcgctacacaatgcactgggtgaagcagcgacctgggcagggactggaatggatcgggtacattaatccaagcaggggatacaccaactacaaccagaagtttaaagacaaggctactctgactaccgataagtcaagctccaccgcatacatgcagctgtctagtctgacatcagaggacagcgccgtgtactattgcgctcgctactatgacgatcattattgtctggattattggggacaggggacaactctgacagtgtcaagcggaggaggaggaagcggaggaggcggctccggcggaggaggctctgacatccagctgactcagtctcccgccattatgtcagcttcccctggcgaaaaagtgaccatgacatgccgggcctcctctagtgtcagctatatgaactggtaccagcagaaatccgggacttctccaaagcgatggatctatgacacctctaaggtggctagtggagtcccctaccggttctccggatctggcagtgggacttcatatagcctgaccatttcaagcatggaggccgaagatgctgcaacctactattgtcagcagtggtcctctaatcccctgaccttcggggctgggactaaactggaactgaaatcatga

SEQ ID NO: 18 - DNA, 1559 bp, Artificial Sequence
  SEQ ID NO18,  Nucleotide sequences for BCMA- CD3 scFv
atgctataaatggctctgcccgtgacagctctgctgctccctctggctctgctgctgcatgctgctaggcccgacatcgtgctgacccagtcccctcctagcctcgccatgtctctgggaaagagagccaccatcagctgtagagcctccgaaagcgtgaccattctcggcagccatctgatccactggtatcagcagaagcccggccaaccccctacactgctgatccagctggccagcaatgtgcagaccggagtgcccgctagattttccggatccggatccagaaccgactttacactgaccatcgaccccgtggaagaggacgacgtggccgtgtactactgtctgcagtctagaaccatccccagaacattcggcggaggcacaaagctggagatcaagggctccacaagcggcagcggcaaacccggcagcggagagggcagcacaaagggccaaatccagctggtgcagagcggccccgaactcaagaagcccggagaaaccgtgaagatcagctgcaaggcctccggctacacattcaccgattactccatcaattgggtcaagagggcccccggcaagggactgaagtggatgggctggattaataccgagacaagagagcccgcctacgcttacgactttagaggaaggttcgccttcagcctcgagacatccgctagcaccgcctatctgcagatcaacaacctcaatacgaggacaccgccacctatttctgtgctctggactactcctatgccatggattactggggacaaggcacaagcgtcacagtgagctccggaggaggaggatccgtcgacgacatcaagctccagcagtccggcgccgaactcgctagacccggagcttccgtcaagatgagctgcaagacctccggatacacattcacaagatacacaatgcactgggtcaaacaaaggcccggccaaggcctcgagtggattggctacatcaacccctctagaggatataccaactacaatcagaaattcaaggacaaagccaccctcacaaccgacaagagcagcagcacagcctacatgcagctgagctctctgacatccgaagacagcgccgtgtattactgcgctagatactatgacgaccactactgtctggactattggggacaaggaacaacactgacagtcagctccggcggcggaggatccggaggcggaggaagcggcggaggaggcagcgacatccagctgacacagtcccccgccattatgagcgcctcccccggcgaaaaggtcaccatgacatgcagagcctccagctccgtcagctatatgaactggtaccagcagaaaagcggcacaagccctaagaggtggatctacgacacctccaaggtcgcttccggagtgccctataggttctccggaagcggatccggaacctcctactctctgacaatctcctccatggaagccgaggacgctgccacctattactgccagcagtggagcagcaatcctctcacctttggcgccggaaccaaactcgagctgaagtcctaatga

SEQ ID NO: 19 - DNA, 1212 bp, Artificial Sequence
  SEQ ID NO19,  Nucleotide sequences for PD-1- ED-hIgG1-Fc
atgcaaattccccaagctccttggcccgtggtctgggccgtgctgcagctgggatggagacccggctggtttctcgactcccccgataggccttggaacccccctaccttttttcccgctctgctggtggtgaccgaaggcgacaacgccaccttcacatgcagcttcagcaacaccagcgagagcttcgtgctcaactggtatagaatgtcccctagcaaccagaccgacaagctggccgccttccccgaggatagatcccaacccggccaagactgcagattcagagtgacccagctgcccaacggaagggatttccacatgtccgtggtcagagctagaaggaatgacagcggaacatacctctgcggcgccatttctctggcccctaaggctcagatcaaggagtctctgagggctgaactgagagtgacagagagaagagccgaagtgcccacagcccacccttcccctagccctagacccgctggccaatttcagacactcgtcgagcccaagagctgcgataagacccacacatgccctccttgtcccgctcccgagctgctcggcggaccctccgtgtttctgtttccccccaaacccaaggacaccctcatgatttctagaacacccgaggtgacatgcgtggtggtggatgtgtcccatgaagaccccgaggtcaagttcaactggtacgtggacggcgtggaggtgcataacgctaagaccaagcctagagaggaacagtataacagcacctatagagtcgtgtccgtgctgacagtgctgcaccaagactggctgaacggcaaagagtataaatgcaaggtcagcaacaaggctctgcccgcccccattgagaagaccatcagcaaggccaagggccagcctagggaacctcaagtgtataccctccctccctctagagaggagatgaccaagaatcaagtgtccctcacatgcctcgtgaaaggcttctaccctagcgacatcgccgtcgaatgggaaagcaacggacagcccgagaacaactacaagaccacaccccccgtgctcgattccgacggcagcttctttctgtactccaagctgaccgtggataagtctagatggcaacaaggcaatgtgttcagctgctccgtcatgcacgaggctctgcacaaccactacacccagaaatctctgtctctgagccccggcaaatgatga

SEQ ID NO: 20 - protein, 12 aa, Artificial Sequence
  Position 1 of the Peptide Array
MAAAKTPVIVVP

SEQ ID NO: 21 - protein, 12 aa, Artificial Sequence
  Position 2 of the Peptide Array
KTPVIVVPVIDR

SEQ ID NO: 22 - protein, 12 aa, Artificial Sequence
  Position 3 of the Peptide Array
IVVPVIDRLPSE

SEQ ID NO: 23 - protein, 12 aa, Artificial Sequence
  Position 4 of the Peptide Array
VIDRLPSETFPN

SEQ ID NO: 24 - protein, 12 aa, Artificial Sequence
  Position 5 of the Peptide Array
LPSETFPNVHEH

SEQ ID NO: 25 - protein, 12 aa, Artificial Sequence
  Position 6 of the Peptide Array
TFPNVHEHINDQ

SEQ ID NO: 26 - protein, 12 aa, Artificial Sequence
  Position 7 of the Peptide Array
VHEHINDQKFDD

SEQ ID NO: 27 - protein, 12 aa, Artificial Sequence
  Position 8 of the Peptide Array
INDQKFDDVKDN

SEQ ID NO: 28 - protein, 12 aa, Artificial Sequence
  Position 9 of the Peptide Array
KFDDVKDNEVMP

SEQ ID NO: 29 - protein, 12 aa, Artificial Sequence
  Position 10 of the Peptide Array
VKDNEVMPEKRN

SEQ ID NO: 30 - protein, 12 aa, Artificial Sequence
  Position 11 of the Peptide Array
EVMPEKRNVVVV

SEQ ID NO: 31 - protein, 12 aa, Artificial Sequence
  Position 12 of the Peptide Array
EKRNVVVVKDDP

SEQ ID NO: 32 - protein, 12 aa, Artificial Sequence
  Position 13 of the Peptide Array
VVVVKDDPDHYK

SEQ ID NO: 33 - protein, 12 aa, Artificial Sequence
  Position 14 of the Peptide Array
KDDPDHYKDYAF

SEQ ID NO: 34 - protein, 12 aa, Artificial Sequence
  Position 15 of the Peptide Array
DHYKDYAFIQWT

SEQ ID NO: 35 - protein, 12 aa, Artificial Sequence
  Position 16 of the Peptide Array
DYAFIQWTGGNI

SEQ ID NO: 36 - protein, 12 aa, Artificial Sequence
  Position 17 of the Peptide Array
IQWTGGNIRNDD

SEQ ID NO: 37 - protein, 12 aa, Artificial Sequence
  Position 18 of the Peptide Array
GGNIRNDDKYTH

SEQ ID NO: 38 - protein, 12 aa, Artificial Sequence
  Position 19 of the Peptide Array
RNDDKYTHFFSG

SEQ ID NO: 39 - protein, 12 aa, Artificial Sequence
  Position 20 of the Peptide Array
KYTHFFSGFCNT

SEQ ID NO: 40 - protein, 12 aa, Artificial Sequence
  Position 21 of the Peptide Array
FFSGFCNTMCTE

SEQ ID NO: 41 - protein, 12 aa, Artificial Sequence
  Position 22 of the Peptide Array
FCNTMCTEETKR

SEQ ID NO: 42 - protein, 12 aa, Artificial Sequence
  Position 23 of the Peptide Array
MCTEETKRNIAR

SEQ ID NO: 43 - protein, 12 aa, Artificial Sequence
  Position 24 of the Peptide Array
ETKRNIARHLAL

SEQ ID NO: 44 - protein, 12 aa, Artificial Sequence
  Position 25 of the Peptide Array
NIARHLALWDSN

SEQ ID NO: 45 - protein, 12 aa, Artificial Sequence
  Position 26 of the Peptide Array
HLALWDSNFFTE

SEQ ID NO: 46 - protein, 12 aa, Artificial Sequence
  Position 27 of the Peptide Array
WDSNFFTELENK

SEQ ID NO: 47 - protein, 12 aa, Artificial Sequence
  Position 28 of the Peptide Array
FFTELENKKVEY

SEQ ID NO: 48 - protein, 12 aa, Artificial Sequence
  Position 29 of the Peptide Array
LENKKVEYVVIV

SEQ ID NO: 49 - protein, 12 aa, Artificial Sequence
  Position 30 of the Peptide Array
KVEYVVIVENDN

SEQ ID NO: 50 - protein, 12 aa, Artificial Sequence
  Position 31 of the Peptide Array
VVIVENDNVIED

SEQ ID NO: 51 - protein, 12 aa, Artificial Sequence
  Position 32 of the Peptide Array
ENDNVIEDITFL

SEQ ID NO: 52 - protein, 12 aa, Artificial Sequence
  Position 33 of the Peptide Array
VIEDITFLRPVL

SEQ ID NO: 53 - protein, 12 aa, Artificial Sequence
  Position 34 of the Peptide Array
ITFLRPVLKAMH

SEQ ID NO: 54 - protein, 12 aa, Artificial Sequence
  Position 35 of the Peptide Array
RPVLKAMHDKKI

SEQ ID NO: 55 - protein, 12 aa, Artificial Sequence
  Position 36 of the Peptide Array
KAMHDKKIDILQ

SEQ ID NO: 56 - protein, 12 aa, Artificial Sequence
  Position 37 of the Peptide Array
DKKIDILQMREI

SEQ ID NO: 57 - protein, 12 aa, Artificial Sequence
  Position 38 of the Peptide Array
DILQMREIITGN

SEQ ID NO: 58 - protein, 12 aa, Artificial Sequence
  Position 39 of the Peptide Array
MREIITGNKVKT

SEQ ID NO: 59 - protein, 12 aa, Artificial Sequence
  Position 40 of the Peptide Array
ITGNKVKTELVM

SEQ ID NO: 60 - protein, 12 aa, Artificial Sequence
  Position 41 of the Peptide Array
KVKTELVMDKNH

SEQ ID NO: 61 - protein, 12 aa, Artificial Sequence
  Position 42 of the Peptide Array
ELVMDKNHAIFT

SEQ ID NO: 62 - protein, 12 aa, Artificial Sequence
  Position 43 of the Peptide Array
DKNHAIFTYTGG

SEQ ID NO: 63 - protein, 12 aa, Artificial Sequence
  Position 44 of the Peptide Array
AIFTYTGGYDVS

SEQ ID NO: 64 - protein, 12 aa, Artificial Sequence
  Position 45 of the Peptide Array
YTGGYDVSLSAY

SEQ ID NO: 65 - protein, 12 aa, Artificial Sequence
  Position 46 of the Peptide Array
YDVSLSAYIIRV

SEQ ID NO: 66 - protein, 12 aa, Artificial Sequence
  Position 47 of the Peptide Array
LSAYIIRVTTEL

SEQ ID NO: 67 - protein, 12 aa, Artificial Sequence
  Position 48 of the Peptide Array
IIRVTTELNIVD

SEQ ID NO: 68 - protein, 12 aa, Artificial Sequence
  Position 49 of the Peptide Array
TTELNIVDEIIK

SEQ ID NO: 69 - protein, 12 aa, Artificial Sequence
  Position 50 of the Peptide Array
NIVDEIIKSGGL

SEQ ID NO: 70 - protein, 12 aa, Artificial Sequence
  Position 51 of the Peptide Array
EIIKSGGLSSGF

SEQ ID NO: 71 - protein, 12 aa, Artificial Sequence
  Position 52 of the Peptide Array
SGGLSSGFYFEI

SEQ ID NO: 72 - protein, 12 aa, Artificial Sequence
  Position 53 of the Peptide Array
SSGFYFEIARIE

SEQ ID NO: 73 - protein, 12 aa, Artificial Sequence
  Position 54 of the Peptide Array
YFEIARIENEMK

SEQ ID NO: 74 - protein, 12 aa, Artificial Sequence
  Position 55 of the Peptide Array
ARIENEMKINRQ

SEQ ID NO: 75 - protein, 12 aa, Artificial Sequence
  Position 56 of the Peptide Array
NEMKINRQILDN

SEQ ID NO: 76 - protein, 12 aa, Artificial Sequence
  Position 57 of the Peptide Array
INRQILDNAAKY

SEQ ID NO: 77 - protein, 12 aa, Artificial Sequence
  Position 58 of the Peptide Array
ILDNAAKYVEHD

SEQ ID NO: 78 - protein, 12 aa, Artificial Sequence
  Position 59 of the Peptide Array
AAKYVEHDPRLV

SEQ ID NO: 79 - protein, 12 aa, Artificial Sequence
  Position 60 of the Peptide Array
VEHDPRLVAEHR

SEQ ID NO: 80 - protein, 12 aa, Artificial Sequence
  Position 61 of the Peptide Array
PRLVAEHRFENM

SEQ ID NO: 81 - protein, 12 aa, Artificial Sequence
  Position 62 of the Peptide Array
AEHRFENMKPNF

SEQ ID NO: 82 - protein, 12 aa, Artificial Sequence
  Position 63 of the Peptide Array
FENMKPNFWSRI

SEQ ID NO: 83 - protein, 12 aa, Artificial Sequence
  Position 64 of the Peptide Array
KPNFWSRIGTAA

SEQ ID NO: 84 - protein, 12 aa, Artificial Sequence
  Position 65 of the Peptide Array
WSRIGTAATKRY

SEQ ID NO: 85 - protein, 12 aa, Artificial Sequence
  Position 66 of the Peptide Array
GTAATKRYPGVM

SEQ ID NO: 86 - protein, 12 aa, Artificial Sequence
  Position 67 of the Peptide Array
TKRYPGVMYAFT

SEQ ID NO: 87 - protein, 12 aa, Artificial Sequence
  Position 68 of the Peptide Array
PGVMYAFTTPLI

SEQ ID NO: 88 - protein, 12 aa, Artificial Sequence
  Position 69 of the Peptide Array
YAFTTPLISFFG

SEQ ID NO: 89 - protein, 7 aa, Artificial Sequence
  aa 11-18 of the H3L Peptides
PVIDRLP

SEQ ID NO: 90 - protein, 11 aa, Artificial Sequence
  aa 30-40 of the H3L Peptides
NDQKFDDVKDN

SEQ ID NO: 91 - protein, 9 aa, Artificial Sequence
  aa 44-52 of the H3L Peptides
PERKNVVVV

SEQ ID NO: 92 - protein, 10 aa, Artificial Sequence
  aa 128-137 of the H3L Peptides
NVIEDITFLR

SEQ ID NO: 93 - protein, 5 aa, Artificial Sequence
  aa 152-156 of the H3L Peptides
QMREI

SEQ ID NO: 94 - protein, 8 aa, Artificial Sequence
  aa 161-168 of the H3L Peptides
KVKTELVM

SEQ ID NO: 95 - protein, 8 aa, Artificial Sequence
  aa 197-204 of the H3L Peptides
NIVDEIIK

SEQ ID NO: 96 - protein, 6 aa, Artificial Sequence
  aa 224-229 of the H3L Peptides
KINRQI

SEQ ID NO: 97 - protein, 8 aa, Artificial Sequence
  aa 249-265 of the H3L Peptides
FENMKPNF

SEQ ID NO: 98 - protein, 7 aa, Artificial Sequence
  Variant of Peptide 1 (Seq No. 89)
AVIDRLP

SEQ ID NO: 99 - protein, 7 aa, Artificial Sequence
  Variant of Peptide 1 (Seq No. 89)
PAIDRLP

SEQ ID NO: 100 - protein, 7 aa, Artificial Sequence
  Variant of Peptide 1 (Seq No. 89)
PVADRLP

SEQ ID NO: 101 - protein, 7 aa, Artificial Sequence
  Variant of Peptide 1 (Seq No. 89)
PVIARLP

SEQ ID NO: 102 - protein, 7 aa, Artificial Sequence
  Variant of Peptide 1 (Seq No. 89)
PVIDALP

SEQ ID NO: 103 - protein, 7 aa, Artificial Sequence
  Variant of Peptide 1 (Seq No. 89)
PVIDRAP

SEQ ID NO: 104 - protein, 7 aa, Artificial Sequence
  Variant of Peptide 1 (Seq No. 89)
PVIDRLA

SEQ ID NO: 105 - protein, 11 aa, Artificial Sequence
  Variant of Peptide 2 (Seq No. 90)
ADQKFDDVKDN

SEQ ID NO: 106 - protein, 11 aa, Artificial Sequence
  Variant of Peptide 2 (Seq No. 90)
NAQKFDDVKDN

SEQ ID NO: 107 - protein, 11 aa, Artificial Sequence
  Variant of Peptide 2 (Seq No. 90)
NDAKFDDVKDN

SEQ ID NO: 108 - protein, 11 aa, Artificial Sequence
  Variant of Peptide 2 (Seq No .90)
NDQAFDDVKDN

SEQ ID NO: 109 - protein, 11 aa, Artificial Sequence
  Variant of Peptide 2 (Seq No. 90)
NDQKADDVKDN

SEQ ID NO: 110 - protein, 11 aa, Artificial Sequence
  Variant of Peptide 2 (Seq No. 90)
NDQKFADVKDN

SEQ ID NO: 111 - protein, 11 aa, Artificial Sequence
  Variant of Peptide 2 (Seq No. 90)
NDQKFDAVKDN

SEQ ID NO: 112 - protein, 11 aa, Artificial Sequence
  Variant of Peptide 2 (Seq No. 90)
NDQKFDDAKDN

SEQ ID NO: 113 - protein, 11 aa, Artificial Sequence
  Variant of Peptide 2 (Seq No. 90)
NDQKFDDVADN

SEQ ID NO: 114 - protein, 11 aa, Artificial Sequence
  Variant of Peptide 2 (Seq No. 90)
NDQKFDDVKAN

SEQ ID NO: 115 - protein, 11 aa, Artificial Sequence
  Variant of Peptide 2 (Seq No. 90)
NDQKFDDVKDA

SEQ ID NO: 116 - protein, 8 aa, Artificial Sequence
  Variant of Peptide 3 (Seq No. 91)
AKRNVVVV

SEQ ID NO: 117 - protein, 8 aa, Artificial Sequence
  Variant of Peptide 3 (Seq No. 91)
EARNVVVV

SEQ ID NO: 118 - protein, 8 aa, Artificial Sequence
  Variant of Peptide 3 (Seq No. 91)
EKANVVVV

SEQ ID NO: 119 - protein, 8 aa, Artificial Sequence
  Variant of Peptide 3 (Seq No. 91)
EKRAVVVV

SEQ ID NO: 120 - protein, 8 aa, Artificial Sequence
  Variant of Peptide 3 (Seq No. 91)
EKRNAVVV

SEQ ID NO: 121 - protein, 8 aa, Artificial Sequence
  Variant of Peptide 3 (Seq No. 91)
EKRNVAVV

SEQ ID NO: 122 - protein, 8 aa, Artificial Sequence
  Variant of Peptide 3 (Seq No. 91)
EKRNVVAV

SEQ ID NO: 123 - protein, 8 aa, Artificial Sequence
  Variant of Peptide 3 (Seq No. 91)
EKRNVVVA

SEQ ID NO: 124 - protein, 10 aa, Artificial Sequence
  Variant of Peptide 4 (Seq No. 92)
AVIEDITFLR

SEQ ID NO: 125 - protein, 10 aa, Artificial Sequence
  Variant of Peptide 4 (Seq No. 92)
NAIEDITFLR

SEQ ID NO: 126 - protein, 10 aa, Artificial Sequence
  Variant of Peptide 4 (Seq No. 92)
NVAEDITFLR

SEQ ID NO: 127 - protein, 10 aa, Artificial Sequence
  Variant of Peptide 4 (Seq No. 92)
NVIADITFLR

SEQ ID NO: 128 - protein, 10 aa, Artificial Sequence
  Variant of Peptide 4 (Seq No. 92)
NVIEAITFLR

SEQ ID NO: 129 - protein, 10 aa, Artificial Sequence
  Variant of Peptide 4 (Seq No. 92)
NVIEDATFLR

SEQ ID NO: 130 - protein, 10 aa, Artificial Sequence
  Variant of Peptide 4 (Seq No. 92)
NVIEDIAFLR

SEQ ID NO: 131 - protein, 10 aa, Artificial Sequence
  Variant of Peptide 4 (Seq No. 92)
NVIEDITALR

SEQ ID NO: 132 - protein, 10 aa, Artificial Sequence
  Variant of Peptide 4 (Seq No. 92)
NVIEDITFAR

SEQ ID NO: 133 - protein, 10 aa, Artificial Sequence
  Variant of Peptide 4 (Seq No. 92)
NVIEDITFLA

SEQ ID NO: 134 - protein, 5 aa, Artificial Sequence
  Variant of Peptide 5 (Seq No. 93)
AMREI

SEQ ID NO: 135 - protein, 5 aa, Artificial Sequence
  Variant of Peptide 5 (Seq No. 93)
QAREI

SEQ ID NO: 136 - protein, 5 aa, Artificial Sequence
  Variant of Peptide 5 (Seq No. 93)
QMAEI

SEQ ID NO: 137 - protein, 5 aa, Artificial Sequence
  Variant of Peptide 5 (Seq No. 93)
QMRAI

SEQ ID NO: 138 - protein, 5 aa, Artificial Sequence
  Variant of Peptide 5 (Seq No. 93)
QMREA

SEQ ID NO: 139 - protein, 8 aa, Artificial Sequence
  Variant of Peptide 6 (Seq No. 94)
AVKTELVM

SEQ ID NO: 140 - protein, 8 aa, Artificial Sequence
  Variant of Peptide 6 (Seq No. 94)
KAKTELVM

SEQ ID NO: 141 - protein, 8 aa, Artificial Sequence
  Variant of Peptide 6 (Seq No. 94)
KVATELVM

SEQ ID NO: 142 - protein, 8 aa, Artificial Sequence
  Variant of Peptide 6 (Seq No. 94)
KVKAELVM

SEQ ID NO: 143 - protein, 8 aa, Artificial Sequence
  Variant of Peptide 6 (Seq No. 94)
KVKTALVM

SEQ ID NO: 144 - protein, 8 aa, Artificial Sequence
  Variant of Peptide 6 (Seq No. 94)
KVKTEAVM

SEQ ID NO: 145 - protein, 8 aa, Artificial Sequence
  Variant of Peptide 6 (Seq No. 94)
KVKTELAM

SEQ ID NO: 146 - protein, 8 aa, Artificial Sequence
  Variant of Peptide 6 (Seq No. 94)
KVKTELVA

SEQ ID NO: 147 - protein, 8 aa, Artificial Sequence
  Variant of Peptide 7 (Seq No. 95)
AIVDEIIK

SEQ ID NO: 148 - protein, 8 aa, Artificial Sequence
  Variant of Peptide 7 (Seq No. 95)
NAVDEIIK

SEQ ID NO: 149 - protein, 8 aa, Artificial Sequence
  Variant of Peptide 7 (Seq No. 95)
NIADEIIK

SEQ ID NO: 150 - protein, 8 aa, Artificial Sequence
  Variant of Peptide 7 (Seq No. 95)
NIVAEIIK

SEQ ID NO: 151 - protein, 8 aa, Artificial Sequence
  Variant of Peptide 7 (Seq No. 95)
NIVDAIIK

SEQ ID NO: 152 - protein, 8 aa, Artificial Sequence
  Variant of Peptide 7 (Seq No. 95)
NIVDEAIK

SEQ ID NO: 153 - protein, 8 aa, Artificial Sequence
  Variant of Peptide 7 (Seq No. 95)
NIVDEIAK

SEQ ID NO: 154 - protein, 8 aa, Artificial Sequence
  Variant of Peptide 7 (Seq No. 95)
NIVDEIIA

SEQ ID NO: 155 - protein, 6 aa, Artificial Sequence
  Variant of Peptide 8 (Seq No. 96)
AINRQI

SEQ ID NO: 156 - protein, 6 aa, Artificial Sequence
  Variant of Peptide 8 (Seq No. 96)
KANRQI

SEQ ID NO: 157 - protein, 6 aa, Artificial Sequence
  Variant of Peptide 8 (Seq No. 96)
KIARQI

SEQ ID NO: 158 - protein, 6 aa, Artificial Sequence
  Variant of Peptide 8 (Seq No. 96)
KINAQI

SEQ ID NO: 159 - protein, 6 aa, Artificial Sequence
  Variant of Peptide 8 (Seq No. 96)
KINRAI

SEQ ID NO: 160 - protein, 6 aa, Artificial Sequence
  Variant of Peptide 8 (Seq No. 96)
KINRQA

SEQ ID NO: 161 - protein, 8 aa, Artificial Sequence
  Variant of Peptide 9 (Seq No. 97)
AENMKPNF

SEQ ID NO: 162 - protein, 8 aa, Artificial Sequence
  Variant of Peptide 9 (Seq No. 97)
FANMKPNF

SEQ ID NO: 163 - protein, 8 aa, Artificial Sequence
  Variant of Peptide 9 (Seq No. 97)
FEAMKPNF

SEQ ID NO: 164 - protein, 8 aa, Artificial Sequence
  Variant of Peptide 9 (Seq No. 97)
FENAKPNF

SEQ ID NO: 165 - protein, 8 aa, Artificial Sequence
  Variant of Peptide 9 (Seq No. 97)
FENMAPNF

SEQ ID NO: 166 - protein, 8 aa, Artificial Sequence
  Variant of Peptide 9 (Seq No. 97)
FENMKANF

SEQ ID NO: 167 - protein, 8 aa, Artificial Sequence
  Variant of Peptide 9 (Seq No. 97)
FENMKPAF

SEQ ID NO: 168 - protein, 8 aa, Artificial Sequence
  Variant of Peptide 9 (Seq No. 97)
FENMKPNA

SEQ ID NO: 169 - protein, 8 aa, Artificial Sequence
  Control peptide for set 3 peptides
EKRNVVVV

SEQ ID NO: 170 - protein, 324 aa, Artificial Sequence
  Mutant H3L amino acid
MAAAKTPVIVVPVAAALPSETFPNVHEHINDQAAADVADAEVMAAKRNVVVAKDDPDHYKDYAFIQWTGGNIRNDDKYTHFFSGFCNTMCTEETKRNIARHLALWDSNFFTELENKKVEYVVIVENDNVIAAIAAAAPVLKAMHDKKIDILQMAAAITGNAVKTEAAADKNHAIFTYTGGYDVSLSAYIIRVTTALNAADEIIKSGGLSSGFYFEIARIENEMKINAQILDNAAKYVEHDPRLVAEHRFAAAAAAAWARIGPATTIRCPGVKNANTAPLISFFGLFDINVIGLIVILFIMFMLIFNVKSKLLWFLTGTFVTAFI

SEQ ID NO: 171 - DNA, 978 bp, Artificial Sequence
  Nucleotide sequences for Mutant H3L
atggctgccgccaaaacccccgtgattgtggtccccgtggccgctgctctgccttccgagacattccccaacgtgcacgaacacatcaatgaccaagctgccgctgacgtggccgacgccgaagtcatggccgctaagagaaacgtggtcgtggccaaggatgaccccgaccactacaaggactatgccttcatccagtggactggtggcaacatcagaaacgacgacaagtacacccatttcttcagcggcttctgcaacaccatgtgtaccgaggagaccaagaggaacatcgctcgtcacctcgccctctgggactccaatttcttcaccgagctggagaacaagaaggtcgagtacgtggtgatcgtggagaacgacaacgtgatcgccgctatcgctgccgccgctcccgttttaaaagccatgcacgacaagaagatcgacattttacagatggccgctgccatcaccggaaacgccgtcaagaccgaggctgccgccgataagaaccacgccatcttcacctacaccggcggatatgacgtgagcctctccgcttacatcattagggtgaccaccgctttaaacgccgccgacgaaatcatcaaatccggaggtttaagctccggcttctacttcgagatcgctcgtatcgagaatgaaatgaagatcaatgcccagattttagataatgccgccaaatacgtggaacatgaccctcgtctggtggctgagcatcgttttgctgctgctgccgctgctgcttgggccagaatcggacccgccaccaccattagatgccccggtgtgaaaaacgccaacaccgcccctttaatttccttcttcggtttattcgacatcaacgtgatcggcctcatcgtgattttattcatcatgttcatgctgatcttcaacgtgaagtccaagttattatggtttttaactggtaccttcgtgaccgccttcatctgataa

SEQ ID NO: 172 - protein, 304 aa, Artificial Sequence
  Mutant D8L amino acid sequence
MPQQLSPINIETKKAISNARLKPLDIHYNESKPTTIQNTGKLAAINFAGGYIAAAFLPNEYVLSSLHIYWGKEDDYGSNHLIDVYKYSGEINLVHWNAKKYSSYEEAAAHDDGLIIISIFLQVLDHKNVYFQKIVNQLDSIRSGNTSAPFDSVFYLDNLLPSKLDYFAYLGTTINHAADAVWIIFPTPINIHSDQASKARTLASSSAHDGKAHYITEAYANAYKLNADTQVYYSGEIIRAATTSPARENYFMRWLSDLRETCFSYYQKYIEENKTFAIIAIVFVFILTAILFFMSRRYSREKQN

SEQ ID NO: 173 - DNA, 918 bp, Artificial Sequence
  Nucleotide sequences for Mutant D8L
atgccccagcaactgtctcccatcaacatcgagaccaagaaggccatttccaacgctcgtctgaagcctttagacatccactacaatgagagcaagcccaccaccatccagaacactggtaagctggccgccatcaactttgccggcggctacatcgccgccgcctttctgcccaacgagtacgtgctcagctctttacacatctattggggcaaagaggacgactacggctccaaccatttaatcgacgtctacaagtattccggcgagatcaatttagtgcactggaacgccaagaagtactccagctacgaagaagccgctgcccacgacgacggactgatcatcatcagcatctttctccaagttctggaccacaagaacgtgtacttccagaagatcgtcaaccagctcgacagcattcgttccggcaatacatccgccccctttgattccgtgttctatttagacaatttactgccctccaagctggactacttcgcctatttaggcaccaccatcaatcacgccgccgatgctgtgtggatcatcttccccacccccattaacattcacagcgatcaagctagcaaggccagaactttagcctccagcagcgctcacgacggcaaggctcactacatcaccgaggcctatgccaacgcctacaagctcaacgccgacacccaagtttactactccggtgagatcattagagctgccacaacctcccccgctcgtgagaactacttcatgaggtggctgtccgatttaagagagacttgtttctcctactatcagaaatacatcgaggagaacaagaccttcgccatcatcgccatcgtgttcgtgttcattttaaccgccattttattcttcatgtctcgtaggtactctcgtgagaagcagaattgataa

SEQ ID NO: 174 - protein, 304 aa, Artificial Sequence
  mutant D8L amino acid sequence (Alternate for Seq No. 6 - only different at position 43)
MPQQLSPINIETKKAISNARLKPLDIHYNESKPTTIQNTGKLLWINFKGGYISGWFLPNEYVLSSLHIYWGKEDDYGSNHLIDVYKYSGEINLVHWNKKKYSSYEEAKKHDDGLIIISIFLQVLDHKNVYFQKIVNQLDSIRSTNTSAPFDSVFYLDNLLPSKLDYFSYLGTTINHYADAVWIIFPTPINIHSDQLSKYRTLSSSSNHDGKTHYITECYRNLYKLNGDTQVYYSGEIIRAATTSPARENYFMRWLSDLRETCFSYYQKYIEENKTFAIIAIVFVFILTAILFFMSRRYSREKQN